SEQUENCE LISTING (46 sequences)

SEQ ID NO: 1 - DNA, 21 bp, Artificial
  ACVRIB exon-skipping oligonucleotide
uggugucagugaccgcaucau

SEQ ID NO: 2 - DNA, 25 bp, Artificial
  ACVRIB exon skipping oligonucleotide
tctatggtgtcagtgaccgcatcat

SEQ ID NO: 3 - DNA, 21 bp, Artificial
  TGFBR1 exon skipping oligonucleotide
gcagugguccugauugcagca

SEQ ID NO: 4 - DNA, 21 bp, Artificial
  TGFBR1 exon-skipping oligonucleotide
aucuguggcagaaucaugucu

SEQ ID NO: 5 - DNA, 25 bp, Artificial
  TGFBR1 exon-skipping oligonucleotid
gcagtggtcctgattgcagcaatat

SEQ ID NO: 6 - DNA, 21 bp, Artificial
  exon skipping oligonucleotide
uaucuugaccgccugagaggg

SEQ ID NO: 7 - DNA, 21 bp, Artificial
  exon-skipping oligonucleotide
aucugcgaucgggacuuugca

SEQ ID NO: 8 - DNA, 20 bp, Artificial
  dystrophin exon-skipping oligonucleotide
ggccaaaccucggcuuaccu

SEQ ID NO: 9 - DNA, 21 bp, Artificial
  ACVR1B exon-skipping oligonucleotide
uggugucagugacugcaucau

SEQ ID NO: 10 - DNA, 22 bp, Artificial
  ACVR1B exon-skipping oligonucleotide
guuugguccccacccucugauu

SEQ ID NO: 11 - DNA, 21 bp, Artificial
  TGFBR1 exon-skipping oligonucleotide
uguacagagguggcagaaaca

SEQ ID NO: 12 - DNA, 21 bp, Artificial
  TGFBR1 exon-skipping oligonucleotide
gcaaugguccugauugcagca

SEQ ID NO: 13 - DNA, 20 bp, Artificial
  dystrophin exon-skipping oligonucleotide
ucaaggaagauggcauuucu

SEQ ID NO: 14 - DNA, 21 bp, Artificial
  siRNA targeting TGFBR1
gggcaguuacuacaacauauu

SEQ ID NO: 15 - DNA, 21 bp, Artificial
  siRNA targeting TGFBR1
uauguuguaguaacugcccuu

SEQ ID NO: 16 - DNA, 21 bp, Artificial
  siRNA targeting ACVR1B
acuaucaacugccguauuauu

SEQ ID NO: 17 - DNA, 21 bp, Artificial
  siRNA targeting ACVR1B
uaauacggcaguugauaguuu

SEQ ID NO: 18 - DNA, 21 bp, Artificial
  ACVR1B exon skipping oligonucleotide
ugacuucaagucucgaugagc

SEQ ID NO: 19 - DNA, 21 bp, Artificial
  TGFBR1 exon skipping oligonucleotide
cuuuguacagagguggcagaa

SEQ ID NO: 20 - DNA, 21 bp, Artificial
  TGFBR1 exon skipping oligonucleotide
ucugugguuuggagcaauauc

SEQ ID NO: 21 - DNA, 25 bp, Artificial
  TGFBR1 exon skipping oligonucleotide
ttgtatctgtggcagaatcatgtct

SEQ ID NO: 22 - DNA, 20 bp, Artificial
  Dmd exon skipping oligonucleotide
ggccaaaccucggcuuaccu

SEQ ID NO: 23 - DNA, 21 bp, Artificial
  AON hA4E2a
uggugcacgcacacagcagag

SEQ ID NO: 24 - DNA, 21 bp, Artificial
  AON hA4E2b
cguguaguuggccuggaggca

SEQ ID NO: 25 - DNA, 20 bp, Artificial
  AON hA4E2c
aguuggccuggaggcagcug

SEQ ID NO: 26 - DNA, 20 bp, Artificial
  AON hA4E2d
acguguaguuggccuggagg

SEQ ID NO: 27 - DNA, 25 bp, Artificial
  AON hA4E2e
ucucacacguguaguuggccuggag

SEQ ID NO: 28 - DNA, 25 bp, Artificial
  AON hA4E2f
ugcaguagucaguguagcagcagug

SEQ ID NO: 29 - DNA, 19 bp, Artificial
  AON hA4E2g
ucugucucacacguguagu

SEQ ID NO: 30 - DNA, 25 bp, Artificial
  AON hA4E2h
uguugcaguagucaguguagcagca

SEQ ID NO: 31 - DNA, 21 bp, Artificial
  AON hA4E6c
gaugagcaauuccaggcuucc

SEQ ID NO: 32 - DNA, 21 bp, Artificial
  AON hA4E6d
ucugcuauggcacacaugcca

SEQ ID NO: 33 - DNA, 22 bp, Artificial
  AON hA4E6e
gcaaugucaauggugucaguga

SEQ ID NO: 34 - DNA, 22 bp, Artificial
  AON hA5E2c
cagagacaaagcagagcccauc

SEQ ID NO: 35 - DNA, 23 bp, Artificial
  AON hA5E2d
cuguggucucugugacagagaca

SEQ ID NO: 36 - DNA, 23 bp, Artificial
  AON hA5E2e
augcuguuguguauaacuuuguc

SEQ ID NO: 37 - DNA, 21 bp, Artificial
  AON hA5E2f
aauauguuguagucacagacc

SEQ ID NO: 38 - DNA, 21 bp, Artificial
  AON hA5E6a
uaugagcaauggcuggcuuuc

SEQ ID NO: 39 - DNA, 22 bp, Artificial
  AON hA5E6b
ugauuucaaaucucuaugagca

SEQ ID NO: 40 - DNA, 22 bp, Artificial
  AON hA5E6c
ugccaguccuaagucugcaaua

SEQ ID NO: 41 - DNA, 21 bp, Artificial
  AON hA5E6d
auaucaaugguaucuguggcu

SEQ ID NO: 42 - DNA, 23 bp, Artificial
  AON hA5E6e
cugugguuuggagcaauaucaau

SEQ ID NO: 43 - DNA, 21 bp, Artificial
  AON hA5E6f
uucccacucugugguuuggag

SEQ ID NO: 44 - DNA, 17 bp, Artificial
  Acvr1b exon 6 skipping sequence confirmation
gcactcaagatacatgg

SEQ ID NO: 45 - DNA, 19 bp, Artificial
  Tgfbr1 exon 2 skipping sequence confirmation
gggcgaaggaaaagcagtc

SEQ ID NO: 46 - DNA, 16 bp, Artificial
  Tgfbr1 exon 6 skipping sequence confirmation
gtacccaaggatacat

(19) United States
(12) Patent Application Publication
(10) Pub. No.: US 2014/0088174 A1
(43) Pub. Date: Mar. 27, 2014

(54) COMPOUNDS AND METHODS FOR ALTERING ACTIVIN RECEPTOR-LIKE KINASE SIGNALING

(71) Applicant: Academisch Ziekenhuis Leiden h.o.d.n. LUMC, Leiden (NL)

(72) Inventors: Peter Abraham Christiaan "T Hoen, Leiden (NL); Wilhelmus Martinus Hendrikus Hoogaars, Leiden (NL); Dwi Utami Kemaladewi, Leiden (NL); Adriana Marie Rus, Leiden (NL); Peter Ten Duke, Leiden (NL)

(73) Assignee: Academisch Ziekenhuis Leiden h.o.d.n. LUMC, Leiden (NL)

(21) Appl. No.: 14/110,036

(22) PCT Filed: Apr. 5, 2012
(86) PCT No.: PCT/NL2012/050230

§ 371 Date: Nov. 27, 2013

(30) Foreign Application Priority Data
Apr. 5, 2011 (EP) 11161190.1

(51) Int. Cl. C12N 15/113

(52) U.S. Cl. CPC C12N 15/1138
USPC 514/44 A; 536/24.5; 435/377; 435/375; 435/320.1; 435/354; 435/366

(57) ABSTRACT

Described are compounds and methods useful in the promotion of muscle growth, the treatment of muscle loss or insufficient muscle growth, and the treatment of fibrotic conditions.

### CROSS-REFERENCE TO RELATED APPLICATIONS

This application is a national phase entry under 35 U.S.C. §371 of International Patent Application PCT/NL2012/050230, filed Apr. 5, 2012, designating the United States of America and published in English as International Patent Publication WO 2012/138223 A2 on Oct. 11, 2012, which claims the benefit under Article 8 of the Patent Cooperation Treaty to European Patent Application Serial No. 11161190.1 filed Apr. 5, 2011.

### TECHNICAL FIELD

The present disclosure relates to biotechnology generally, and more specifically to compounds and methods useful in the promotion of muscle growth, the treatment of muscle loss or insufficient muscle growth, and the treatment of fibrotic conditions.

### BACKGROUND

The transforming growth factor beta (TGF-beta) protein superfamily consists of more than 30 members including TGF-beta, bone morphogenetic protein (BMP), and growth differentiation factors (GDFs), e.g., myostatin, activins, and nodal-related proteins. These proteins control diverse cellular processes such as control of homeostasis, cell growth and differentiation in multiple adult tissues.

Members of the TGF-beta family are produced as large precursor proteins and elicit their biological responses as secreted dimers. These dimeric ligands initially bind and assemble heterotetrameric complex of transmembrane serine/threonine kinase receptors, designated as the type I and type II receptors. The receptor assembly leads to phosphorylation-dependent activation of the type I receptor in the Glycine-Serine rich (GS) domain. Once in the activated state, the type I receptor will phosphorylate the cytoplasmic intracellular Smad receptor-regulatory proteins. Upon association with Smad, the Smad complex will be translocated into the nucleus where it regulates the transcription of several target genes (Moustakas et al. (2001), J. Cell Sci. 114, 4359-4369; and Y. Shi and J. Massague (2003), Cell 113, 685-700).

Each TGF-beta family member uses a combination of the type II and type I receptors to exert its biological functions. Activin initiates signaling by binding to Activin type II (ACVRII) or Activin type IIb (ACVRIIB) receptors and type I activin receptor-like kinase (ACTRIB/ALK-4/EntrezGene number 91). Myostatin, like Activin, can bind to ACVRII, ACVRIIB and ACVR1B, but also to TGF-beta-receptor 1 (TGFBR1/ALK-5/EntrezGene number 7046). TGF-beta itself mainly uses TGF-beta type II receptor (TGFBRII) and TGFBR1 or ACVRL1 (also known as ALK1 and having EntrezGene number 94). The ACVR2B receptor is known to interact with several TGF-beta family ligands including Activin A, Inhibin A/B, Nodal, BMP2, BMP6/7, GDF5, myostatin, and GDF11. Ligands for the ACVR1B receptor include myostatin, activins, inhibins, Nodal, GDF1, GDF3 and GDF11. Ligands for TGFBR1 include myostatin, TGFB1, TGFB 2 and TGFB 3. Thus, activation or inhibition of any of these receptors can lead to a number of effects due to interactions with various ligands and different type I receptors.

The specificity of the downstream signaling is determined by the activated type I receptor. ACVR1B and TGFBR1 phosphorylate Smad2 and Smad3, while other type I receptors such as ACVRL1 (also known as ALK1 and having EntrezGene number 94), ACVR1 (also known as ALK2 and having EntrezGene number 90), BMPR1A (also known as ALK3 and having EntrezGene number 567), BMPR1B (also known as ALK6 and having EntrezGene number 658), and ACVR1C (also known as ALK7 and having EntrezGene number 130399) phosphorylate Smad1, Smad5, and Smad8.

In addition, the presence of accessory receptor or type III receptor can either stabilize or decrease the affinity toward the type I/II receptor complex, thereby regulating how specific these cytokines signal. Furthermore, the expression levels of these receptors control the cell type-specific functions of the cytokines. One example is endoglin (Eng), a co-receptor for TGF-beta, which is highly expressed in endothelial cells and determines in these cells whether TGF-beta signals via TGFBR1 as opposed to the functionally and structurally distinct type I receptor ACVRL1 (M. J. Goumans, et al. (2003) Mol. Cell. 12, 817-828 and F. Lebrin, et al. (2004) EMBO J. 23, 4018-4028). Another example is betaglycan (TGFBR3) that can bind TGF-beta and enhances its affinity towards TGFBR2 and TGFBR1(Lopez-Casillas, et al. (1993), Cell 73, 1435-1444). The EGF-CFC family of proteins can also act as accessory receptors (Cheng et al. (2003) Genes Dev. 17:31-36). These proteins contain epidermal growth factor (EGF) and “Cripto-FRL1-Cryptic” (CFC) domains and members in human include TDGF1 (also known as Cripto and having EntrezGene number 6997), and CFC1 (also known as Cryptic and having EntrezGene number 55997).

To further achieve proper signaling in complex multicellular systems, many structurally diverse secreted factors bind directly to TGF-beta family members and regulate their interactions with their cognate receptors. These soluble regulators bind to ligands with different affinities and display differential interactions with cell surface molecules. Examples of these regulators are Follistatin (FST) and Follistatin-related gene (FLRG).

FST can bind to Activin and masks the type I and type II receptor binding sites of Activin, thereby abrogating Activin signaling (Harrington 2006). FST also interacts directly with myostatin and prevent the inhibitory effect of myostatin on the limb muscle development (Amthor 2004). FLRG, which contains two FS domains, inhibits the activities of activin and BMPs in cell culture (Tsuchida 2000, Scheneyer 2001). In addition, it has been shown to form a complex with myostatin in mouse and human serum and inhibit myostatin function in a reporter assay (J. J. Hill 2002).

In addition to the soluble regulators, secreted TGF-beta ligands also encounter various extracellular matrix (ECM) proteins. Upon binding the TGF-beta family members, these ECM proteins can either present them in the proximity of the cognate receptors or sequester them away. Examples of these ECM proteins are decorin, biglycan and perlecan, which all have differential affinities for the different members of the TGF-beta family. In summary, it takes a balance among sequestration, storage and presentations of ligands to regulate TGF-beta family signaling.

TGF-beta family members exert overlapping biological functions. One example of such overlap is in the mesenchymal differentiation process (R. Derynck and R. J. Akhurst (2007), Nat. Cell Biol. 9, 1000-1004). Both TGF-beta and myostatin inhibit differentiation toward myogenic lineage (S. J. Lee (2004), Annu. Rev. Cell Dev. Biol. 20, 61-86). Furthermore, both ligands play a role in differentiation toward fibroblast, proliferation and transformation into myofibroblast (R. Derynck and R. J. Akhurst (2007), Nat. Cell Biol. 9, 1000-1004). In addition, differentiation toward adipogenic lineage is also regulated by TGF-beta and indirectly by myostatin (A. Rebbapragada, et al. (2003), Mol. Cell. Biol. 23, 7230-7242). However, as both TGF-beta and myostatin have the potential to act through multiple type I and type II receptors, and the biological function of each of these receptors can overlap depending on the cell type where each receptor is expressed, the contribution of each individual receptor to the biological functions of TGF-beta and myostatin signaling in any given cell type is not known.

The complexity of the TGF-beta superfamily signaling pathway complicates the ability to predict what effects will be realized by blocking individual components of the pathway in a given cell. In view of this complexity, there remains a need in the art for tools to specifically target the individual receptors in order to identify the in vivo function of individual receptors. Elucidation of the biological role of the receptors together with their effect (or lack of effect) in respective cell types offers new therapeutic treatments in a variety of disorders as well as the potential to develop specific treatments that minimize off-target effects.

### SUMMARY OF THE DISCLOSURE

Provided is a method for promoting muscle growth or treating muscle loss or insufficient muscle growth in an animal, comprising administering to the animal an effective amount of a compound that specifically reduces the amount or activity of ACVR1B protein and/or a compound that specifically reduces the amount or activity of TGFBR1 protein in a cell of the animal. Preferably, the muscle loss or insufficient muscle growth is a symptom of muscular dystrophy, more preferably, Duchenne Muscular dystrophy (DMD) or Becker Muscular Dystrophy. Preferably, the method further comprises dystrophin exon skipping, such as by administering a, dystrophin exon-skipping nucleic acid. Preferably, the amount or activity of ACVR1B protein is reduced in a cell of the myogenic lineage, preferably, a muscle stem cell, a muscle fiber, or a cell expressing MYOD1. Preferably, the amount or activity of TGFBR1 protein is reduced in a cell that is not part of the myogenic lineage. Preferably, a member of the EGF-CFC gene family, preferably TDGF1, is expressed in the cell and a compound that specifically reduces the amount or activity of ACVR1B protein is provided to decrease myostatin signaling. Preferably, a member of the EGF-CFC gene family, preferably TDGF1, is not expressed in the cell and a compound that specifically reduces the amount or activity of TGFBR1 protein is provided to decrease myostatin signaling.

In one aspect, provided is a method for stimulating differentiation of a cell in the myogenic lineage comprising providing the cell with a compound that specifically reduces the amount or activity of ACVR1B protein in the cell or providing the cell with a compound that specifically reduces the amount or activity of TGFBR1 protein in the cell. Preferably, a member of the EGF-CFC gene family, preferably TDGF1, is expressed in the cell and a compound that specifically reduces the amount or activity of ACVR1B protein is provided to decrease myostatin signaling. Preferably, a member of the EGF-CFC gene family, preferably TDGF1, is not expressed in the cell and a compound that specifically reduces the amount or activity of TGFBR1 protein is provided to decrease myostatin signaling.

In one aspect, provided is a method for inhibiting, decreasing and/or preventing the development of fibrosis in an animal comprising administering to the animal in need thereof an effective amount of a compound that specifically reduces the amount or activity of TGFBR1 protein in a cell of the animal, preferably wherein the fibrosis is selected from muscle, pulmonary, liver, or cardiac fibrosis, more preferably wherein the fibrosis is selected from muscle fibrosis. Preferably, a member of the EGF-CFC gene family, preferably TDGF1, is not expressed in the cell. Preferably, the method further comprises administering to the animal in need thereof an effective amount of a compound that specifically reduces the amount or activity of ACVR1B protein in a cell of the animal. The two compounds may be administered separately, sequentially, or simultaneously, as described further herein.

In one aspect, provided is a method for inhibiting, decreasing, and/or preventing the development of fibrosis in an animal, the method comprising administering to the animal in need thereof an effective amount of a compound that specifically reduces the amount or activity of ACVR1B protein in a cell of the animal, preferably wherein the fibrosis is selected from muscle, pulmonary, liver, or cardiac fibrosis, more preferably wherein the fibrosis is selected from muscle fibrosis.

In one aspect, provided is a method for decreasing the proliferation of fibroblast cells and/or differentiation of cells toward myofibroblast phenotype comprising providing the cells with a compound that specifically reduces the amount or activity of TGFBR1 protein in the cell, preferably wherein the fibroblast cell is a myofibroblast. Preferably, a member of the EGF-CFC gene family, preferably TDGF1, is not expressed in the cell.

In one aspect, provided is a method for silencing myostatin and/or activin signaling in a cell that is permissive to myostatin and/or activin signaling comprising selecting a cell that expresses a) ACVR2A and/or ACVR2B, b) ACVR1B, and c) TGFBR1 and providing to the cell an effective amount of a compound that specifically reduces the amount or activity of ACVR1B protein and/or a compound that specifically reduces the amount or activity of TGFBR1 protein in the cell. Preferably, the cell is a cell in the myogenic lineage, preferably, a muscle stem cell, a muscle fiber, or a cell expressing MYOD1 and ACVR1B is silenced. Preferably, the cell is not a cell in the myogenic lineage and TGFBR1 is silenced. Preferably, the cell expresses a member of the EGF-CFC gene family, preferably TDGF1, and ACVR1B is silenced. Preferably, the cell does not express a member of the EGF-CFC gene family, preferably TDGF1, and TGFBR1 is silenced.

In one aspect, provided is a method for silencing TGF-beta signaling in a cell that is permissive to TGF-beta signaling comprising selecting a cell that expresses TGFBR1 and a member of the EGF-CFC gene family, preferably TDGF1, and silencing TGFBR1 in the cell. Preferably, the cell is a cell in the myogenic lineage, preferably, a muscle stem cell, a muscle fiber, or a cell expressing MYOD1 and TGFBR1 is silenced.

In one aspect, provided is a compound that specifically reduces the amount or activity of ACVR1B protein in a cell of an animal selected from an oligonucleotide, as described herein, comprising between 15 and 100, preferably between 15 and 40, nucleotides complementary to an ACVR1B pre-mRNA, a vector comprising the oligonucleotide, and a nucleic acid comprising the oligonucleotide, preferably, operably linked to a promoter sequence.

In one aspect, provided is a compound that specifically reduces the amount or activity of TGFBR1 protein in a cell of an animal selected from an oligonucleotide, as described herein, comprising between 15 and 100, preferably between 15 and 40, nucleotides complementary to a TGFBR1 pre-mRNA, a vector comprising the oligonucleotide, and a nucleic acid comprising the oligonucleotide, preferably, operably linked to a promoter sequence.

Preferably, the compound that specifically reduces the amount or activity of ACVR1B protein is an oligonucleotide comprising between 15 and 40 nucleotides complementary to an ACVR1B pre-mRNA, as described herein. Preferably, the compound that specifically reduces the amount or activity of TGFBR1 protein is an oligonucleotide comprising between 15 and 40 nucleotides complementary to a TGFBR1 pre-mRNA, as described herein. Preferably, the compound is formulated to be administered to an animal in a therapeutically effective amount. Preferably, the compound is formulated to be administered to an animal in conjunction with additional oligonucleotides as described herein.

As is understood by a skilled person, an oligonucleotide that is complementary to a pre-mRNA is also known as having the reverse complement sequence of the pre-mRNA.

In one aspect, provided is an oligonucleotide comprising between 15 and 40 nucleotides complementary to an ACVR1B pre-mRNA, wherein the oligonucleotide binds ACVR1B pre-mRNA and, preferably, modulates splicing of the mRNA. Preferably, the oligonucleotide induces skipping of exons 2, 4, 6 or 7; more preferably, the oligonucleotide induces skipping of exons 4, 5, or 7; even more preferably, the oligonucleotide induces skipping of exon 6. Preferably, the oligonucleotide comprises a sequence selected from SEQ ID NOS:1, 2, 9 or 10. In one aspect, the oligonucleotide is used for increasing muscle mass, muscle growth, muscle differentiation, or decreasing or preventing muscle loss, preferably preventing muscle loss associated with Duchenne Muscular dystrophy. Preferably, the oligonucleotide is used in conjunction with an oligonucleotide comprising between 15 and 40 nucleotides complementary to a TGFBR1 pre-mRNA, and/or with a dystrophin exon-skipping nucleic acid.

In one aspect, provided is an oligonucleotide comprising between 15 and 40 nucleotides complementary to a TGFBR1 pre-mRNA, wherein the oligonucleotide binds TGFBR1 pre-mRNA and, preferably, modulates splicing of the mRNA. Preferably, the oligonucleotide induces skipping of exons 2, 4, 6 or 7; more preferably, the oligonucleotide comprises a sequence selected from SEQ ID NOS:3, 4, 5, 11, and 12. In one aspect, the oligonucleotide is used for increasing muscle mass, muscle growth, muscle differentiation, or decreasing or preventing muscle loss, preferably preventing muscle loss associated with Duchenne Muscular dystrophy. In one aspect, the oligonucleotide is used for inhibiting, decreasing and/or preventing the development of fibrosis, and/or decreasing the proliferation of fibroblasts, and/or decreasing the differentiation of cells into fibroblasts, preferably for preventing fibrosis associated with Duchenne Muscular dystrophy.

In one aspect, provided is a composition or a kit of parts comprising an oligonucleotide comprising between 15 and 40 nucleotides complementary to an ACVR1B pre-mRNA, wherein the oligonucleotide binds ACVR1B pre-mRNA, and/or an oligonucleotide comprising between 15 and 40 nucleotides complementary to a TGFBR1 pre-mRNA, wherein the oligonucleotide binds TGFBR1 pre-mRNA. Preferably, the composition further comprises a dystrophin exon-skipping nucleic acid. Preferably, the composition further comprises a pharmaceutically acceptable excipient.

In one aspect, provided is a nucleic acid molecule that specifically reduces the amount or activity of ACVR1B protein, the nucleic acid comprising an oligonucleotide comprising or consisting of between 15 and 40 nucleotides complementary to an ACVR1B pre-mRNA, wherein the oligonucleotide binds ACVR1B pre-mRNA. In a further aspect, the disclosure provides a nucleic acid that specifically reduces the amount or activity of TGFBR1 protein comprising an oligonucleotide comprising or consisting of between 15 and 40 nucleotides complementary to a TGFBR1 pre-mRNA, wherein the oligonucleotide binds TGFBR1 pre-mRNA. Preferably, the oligonucleotide is operably linked to a promoter sequence. Preferably, the promoter sequence is a Pol II or Pol III promoter. Preferably, the nucleic acid comprises a vector sequence.

In one aspect, provided is a vector comprising the oligonucleotide comprising between 15 and 40 nucleotides complementary to an ACVR1B pre-mRNA and/or the oligonucleotide comprising between 15 and 40 nucleotides complementary to a TGFBR1 pre-mRNA, wherein the oligonucleotide binds its respective target pre-mRNA. Preferably, the vector is a viral vector, more preferably, the viral vector is an adeno-associated viral vector.

In one aspect, provided is a cell comprising a) an oligonucleotide comprising between 15 and 40 nucleotides complementary to an ACVR1B pre-mRNA and/or an oligonucleotide comprising between 15 and 40 nucleotides complementary to TGFBR1 pre-mRNA; b) a vector as disclosed herein comprising the oligonucleotide; or c) a nucleic acid as disclosed herein comprising the oligonucleotide. In one aspect, the disclosure provides a non-human animal, preferably a non-human mammal, comprising the cell.

### BRIEF DESCRIPTION OF THE DRAWINGS

FIG. 1. Knockdown of ACVR1B and TGFBR1. 100 nM of siRNA targeting ACVR1B (siAcvr1b), TGFBR1 (siTgfbr1) or non-targeting siRNA was transfected into the C2C12 mouse myoblast (Panel A) and C3H10 T1/2 mouse mesenchymal stem cells (Panel B). Forty-eight hours post-transfection, the knockdown level was assessed by QRT-PCR on ACVR1B transcript (white bars) and TGFBR1 transcript (black bars). The experiments were repeated at least three times and the representative measurements were plotted. Each experiment was performed in triplicate and the measurements were averaged and the standard deviations were calculated.

FIG. 2. Cell-type specific utilization of type I receptor C2C12 (Panel A) and C3H10 T1/2 (Panel B) cells were co-transfected with (CAGA)₁₂-luciferase and CMV-Renilla luciferase plasmids, with combination of siRNAs targeting ACVR1B (siAcvr1b) or TGFBR1 (siTgfbr1) at 100 nM concentration. Lipid (mock) and non-targeting siRNA (siCtrl) served as controls. Twenty-four hours post-transfection, the cells were serum starved overnight. All conditions were stimulated with 500 ng/ml myostatin (gray bars) or 1 ng/ml (black bars) for eight hours. Firefly luciferase signals were measured and corrected for renilla luciferase signals. The experiments were performed in triplicate. The readings were corrected for non-stimulated condition and plotted as fold inductions±standard deviations of the triplicate experiments.

FIG. 3. Expression and knockdown of myostatin (putative) receptors. Endogenous expression of myostatin type II (Acvr2b) and type I receptors (ACVR1B (ALK4) and TGFBR1 (ALK5)) (FIG. 3A) in C2C12 myoblast (white bars), endothelial-derived (grey bars) and mesenchymal stem cells (black bars). siRNA targeting multiple TGF-beta co-receptors were transfected into C2C12 myoblast at 100 nM concentrations, except siDcn and siTdgf1 which were transfected at both 100 and 200 nM concentrations (FIG. 3B). RNA was isolated 48 hours post-transfection for quantitative PCR (QPCR) analysis. Tdgf1 expression levels in different cell types were assessed by QPCR. Each QPCR measurement was performed in triplicate and the graphs represent the averages±standard deviations.

FIG. 4. The effects of knockdown of different co-receptors in myostatin- and TGF-beta-induced CAGA luciferase activity. C2C12 myoblast (Panels A, C) and C3H10 T1/2 mesenchymal cells (Panels B, D) were transfected with CAGA-luciferase reporter construct, Renilla-luciferase and different siRNA at 100 nM concentration (200 nM for siRNA targeting Dcn and Tdgf1). Upon overnight starvation, the cells were stimulated for eight hours with 500 ng/ml myostatin (gray bars) or 1 ng/ml TGF-beta (black bars). After correction to the renilla for transfection efficiency, the luciferase activity was compared to the non-stimulated condition and plotted as fold induction. Each experiment was performed in triplicate. The graphs represent the average of three independent experiments. Error bars represent the standard deviations of the mean.

FIG. 5. Specific expression of TDGF1 measured by QPCR (FIG. 5A) and Western blot (FIG. 5B). Lane 1: 3T3-L1; Lane 2: C3H10 T1/2; Lane 3: C2C12 myoblast; (+): 293T cells overexpressing TDGF1.

FIG. 6. Activin- and myostatin-induced A3-luciferase C2C12 were transfected with A3-Luciferase, renilla luciferase and FAST-1 constructs, together with 200 nM of siRNA targeting Tdgf1 or controls (FIG. 6A). 293T cells were transfected with A3-Luciferase, renilla luciferase, FAST-1, Acvr2b, ACVR1B constructs and either wild-type, delta EGF or delta CFC TDGF1 plasmids (FIGS. 6B, 6C). Upon overnight serum starvation, the cells were stimulated with 20 ng/ml Activin A (FIG. 6A, 6B) or 500 ng/ml myostatin (FIG. 6C) for eight hours. After correction to the renilla for transfection efficiency, the luciferase activity was compared to the non-stimulated condition and plotted as fold induction. Each experiment was performed in triplicate and repeated at least twice. The representative experiment was shown as average±standard deviations. C2C12 and C3H10 T1/2C cells were transfected with the indicated siRNAs and stimulated with 20 ng/ml activin A for 45 minutes following an overnight starvation. Protein lysates were isolated and analyzed for Smad2 phosphorylation by Western blotting (FIG. 6D).

FIG. 7. ACVR1B and TDGF1 knockdown enhances myoblast differentiation. C2C12 cells were grown on chamber slides for immunofluorescence staining (FIG. 7A) or 24-well plates for RNA analysis (FIG. 7B). siRNA targeting Tdgf1 was transfected at 200 nM concentrations, whereas 100 nM siRNA targeting ACVR1B and TGFBR1 were co-transfected with control siRNA to equalize the final concentration to 200 nM. The cells were transfected and fused at the same time. The cells were fixed at the initial (d0), d2, d3, d5 and d7 time points for immunostaining (FIG. 7A). Red: desmin, green: myosin, blue: DAPI. RNA was isolated at d0, d2 and d5 to assess the knockdown levels and the expression of differentiation marker Myog (FIG. 7B).

FIG. 8. Proposed mechanism of co-receptor regulated-myostatin signaling. TDGF1 (red) is present in myoblasts (Panels A, B) but not in non-myoblasts (Panels C, D). The CFC domain of TDGF1 is associated with ACVR1B (ALK4), thus facilitating the recruitment of ACVR1B for myostatin signaling (A). Upon down-regulation of TDGF1 by siRNA, myostatin signaling is abrogated (Panel B). When TDGF1 is absent or expressed under a certain threshold, myostatin uses TGFBR1 (ALK5) as its main type 1 receptor (Panel C). A TGF-beta co-receptor betaglycan (black) might be another player for myostatin signaling in the cells' environment when TDGF1 is absent (Panel D).

FIG. 9. Antisense oligonucleotides-mediated Acvr1b and Tgfbr1 exon skip. Schematic overview of ACVR1B/TGFBR1 transcript and protein (FIG. 9A). AONs were designed to target exon 2, which encodes for ligand binding domain and exon 6, which encodes for part of the kinase domain. Note that skipping exon 6 will result in an out-of-frame transcript (asterisk, *) and terminate the translation into protein. The AONs were transfected into C2C12 cells at the indicated concentrations and the RNA was isolated 48 hours post-transfection for RT-PCR analysis (FIG. 9B). Sequence analysis confirmed the exclusion of either exon 2 or exon 6 after AON-transfection in C2C12 cells (FIG. 9C) (SEQ ID NOS:44-46).

FIG. 10. Quantification of ACVR1B and TGFBR1 full-length mRNA levels specific at the skipped domain using real-time quantitative PCR. Specific down-regulation of the full-length transcript is observed upon antisense-oligo nucleotide-mediated exon skipping of ACVR1B exon6 (Panel A), TGFBR1 exon2 (Panel B) and TGFBR1 exon6 (Panel C). A4E6=ACVR1B exon6 antisense oligonucleotide; A5E2=TGFBR1 exon2 antisense oligonucleotide; A5E6=TGFBR1 exon6 antisense oligonucleotide; NT=non-transfected; CTRL=control antisense oligonucleotide; LY=small molecule kinase inhibitor of ACVR1B/TGFBR1/ACVR1C.

FIG. 11. TGFBR1 but not ACVR1B exon-skipping oligonucleotides (200 nM) specifically abrogated TGF-beta-induced CAGA activities in C2C12 myoblast (FIG. 11A) and C3H10 T1/2 mesenchymal stem cells (FIG. 11B). See FIG. 4 for protocol luciferase assay. A4E6=ACVR1B exon6 antisense oligonucleotide; A5E2=TGFBR1 exon2 antisense oligonucleotide; A5E6=TGFBR1 exon6 antisense oligonucleotide; NT=non-transfected; CTRL=control antisense oligonucleotide; LY=small molecule inhibitor of ACVR1B/TGFBR1/ACVR1C.

FIG. 12. ACVR1B but not TGFBR1 exon-skipping oligonucleotides (200 nM) specifically abrogated myostatin-induced CAGA activity in C2C12 myoblast (FIG. 12A) and TGFBR1 exon-skipping oligonucleotides abrogated myostatin-induced CAGA activity in C3H10 T1/2 mesenchymal stem cells (FIG. 12B). Myostatin-induced Smad2 phosphorylation in the absence or presence of each AON was also determined in C2C12 myoblast (FIG. 12C) and C3H10 T1/2 mesenchymal stem cells (FIG. 12D). See FIG. 4 for protocol luciferase assay. A4E6=ACVR1B exon6 antisense oligonucleotide; A5E2=TGFBR1 exon2 antisense oligonucleotide; A5E6=TGFBR1 exon6 antisense oligonucleotide; NT=non-transfected; CTRL=control antisense oligonucleotide; LY=small molecule inhibitor of TGFBR1/ACVR1B/ACVR1C. Smad2 phosphorylation can be induced by addition of Activin A in a dose-dependent response in C2C12 myoblast and C3H10 T1/2 mesenchymal stem cells. Activin A-induced Smad2 phosphorylation in the absence of presence of 200 nM of each AON was assessed in C2C12 cells by Western blotting (FIG. 12E). AON-mediated ACVR1B inhibition can reduce Activin-induced Smad2 phosphorylation in C2C12.

FIG. 13. C2C12 myoblasts were grown to ˜80% confluency and differentiation was induced by switching the medium to low serum. After one day of differentiation, cells were transfected with 200 nM of CTRL, A4E6, A5E2, A5E6 AONs and allowed to further differentiate. Immunofluorescent staining for desmin (red), myosin (green) and DAPI (blue) was performed at the indicated time points to assess the myogenic differentiation.

FIG. 14. AON-mediated ACVR1B or TGFBR1 knockdown inhibits TGF-beta-induced fibroblast proliferation and fibrotic responses. 3T3-L1 pre-adipocytes with fibroblast properties were used to assess the effect of myostatin and TGF-beta in fibroblast proliferation (FIG. 14A). The cells were starved using 2% BSA for four hours prior to stimulation with 1 ng/ml TGF-beta (red dotted line), 750 ng/ml myostatin (black line) or vehicle (non-stimulated (NS); gray line). Absorbance at 490 nm (OD490) was used as a measure of cells' growth or viability. The effect of AON was also assessed in parallel (FIG. 14B). Two hundred nM of A4E6 (pink), A5E2 (black, dotted), A5E6 (red) or Control (green) AON was transfected at d and the cell growth was measured over time. Non-transfected (NT) cells were included as control. Primary fibroblast was isolated from skin of hDMD mice and cultured in the 0, 1 or 5 ng/ml of TGF-beta for four days (FIG. 14C). The cells were fixed and stained for alpha-Smooth Muscle Actin (aSMA, green) and DAPI (blue). Primary fibroblasts isolated from gastrocnemius muscles of wild-type mice were grown to ˜60-70% confluency (d0) and transfected with 200 nM of CTRL, A4E6 or A5E2 AONs. Proteins were isolated at the indicated time points and analyzed for the expression of the myofibroblast maker α-smooth muscle actin, and fibroblast markers fibronectin and vimentin by Western blot and quantified densitometrically using actin as loading controls (FIG. 14D).

FIG. 15. Intramuscular injection of exon-skipping AONs for ACVR1B and TGFBR1 evaluated at four days after injection. Four times consecutive injections were performed with 40 μg of ALK5E2 or ALK4E6 AONs into the triceps muscle of mdx mice (n=6 mdx/group). Equal amount of Scrambled ALK5 (ScA5) or scrambled ALK4 (ScA4) AONs were injected into the contralateral triceps as controls. The mice were sacrificed four days after the last injection. The triceps were sectioned to isolate RNA and proteins. FIG. 15A) QPCR analysis was performed to assess the expression of ACVR1B, TGFBR1, myogenic and fibrotic genes, normalized to Gapdh. Statistical analysis was performed using paired t-test. **p<0.01. FIG. 15B) Western blot analysis for Serpinel and pSmad2 were performed. The actin showed equal loading of the proteins. The results from five representative mice from each group were shown. FIG. 15C) Protein lysates were analyzed for SERPINE1 and phosphorylated SMAD2 by Western blot and quantified densitometrically using actin as loading control. FIG. 15D) Intramuscular injection of exon-skipping AONs for ACVR1B and TGFBR1 evaluated at ten days after injection. Mice were sacrificed ten days after the last injection. QPCR analysis was performed to assess the expression of ACVR1B, TGFBR1, myogenic and fibrotic genes, normalized to Gapdh. Statistical analysis was performed using paired t-test. *p<0.05; **p<0.01.

FIG. 16. Acvr1b/Tgfbr1 exon skipping after intramuscular injections in mdx mice. The triceps of mdx mice (n=6 mdx/group) were injected with combinations of A4E6 and DMD AONs, whereas the contralateral triceps received a combination of DMD and ScrA4 AONs. Another group of mdx mice received a combination of A5E2 and DMD AONs in the triceps muscles, and similarly, the scrambled ALK5 and DMD AONs were combined and injected in the contralateral muscles as control. RT-PCR showed combined skipping of DMD and either ACVR1B (ALK4) or TGFBR1 (ALK5).

FIG. 17. Systemic administration of exon-skipping AONs for ACVR1B and TGFBR1. 2×6 mg/kg AONs were injected daily intravenously into the tail vein of mdx mice. The mice were sacrificed four days after the last injection. RNA from different tissues were isolated and analyzed by RT-PCR to detect ACVR1B exon 6 skip and TGFBR1 exon 2 skip. Skip levels were quantified as skip % using Agilent DNA 1000 Chip. Tr=triceps; Gc=gastrocnemius; TA=tibialis anterior; Sol=soleus; Q=quadriceps; Dia=diaphragm; Liv=liver; Kid=kidney; H=heart; Lu=Lung; Fat; Aor=aorta; Br=brain; Bo=bone; (+)=positive control

FIG. 18. Immortalized human myoblast cells were transfected with 200 nM of control, hA5E2a or hA5E2b AONs. RNA was isolated two days post-transfection for RT-PCR analysis.

FIG. 19. AON-mediated exon skipping in the triceps following systemic administration. Mdx mice were intravenously injected with PBS, ACVR1B (ALK4E6, A4E6), TGFBR1 (ALK5E2, A5E2) or a combination of ALK4E6 and ALK5E2 AONs (n=6 per group) weekly for six weeks and sacrificed four days after the last injection. Triceps were isolated and analyzed for detection of exon-skip products by RT-PCR (FIG. 19A). qPCR was performed to quantify the expression of Acvr1b, Tgfbr1 (FIG. 19B), Myog, Collal and α-SMA (FIG. 19C), corrected to Gapdh as a housekeeping gene. Protein lysates were analyzed for SERPINE1, α-SMA and phosphorylated SMAD2 levels by Western blot and quantified densitometrically using ACTIN as loading control (FIG. 19D). Statistical analysis was performed using the Student's t-test. * p<0.05, ** p<0.01

FIG. 20. AON-mediated exon skipping in the diaphragm following systemic administration. Mdx mice were intravenously injected with PBS, ACVR1B (ALK4E6, A4E6), TGFBR1 (ALK5E2, A5E2), or combination of ALK4E6 and ALK5E2 AONs (n=6 per group) weekly for six weeks and sacrificed four days after the last injection. Diaphragms were isolated and analyzed for detection of exon-skip products by RT-PCR (FIG. 20A). QPCR was performed to quantify the expression of ACVR1B, TGFBR1 (FIG. 20B), Myog, Col1a1 and α-SMA (FIG. 20C), corrected to Gapdh as housekeeping gene. Protein lysates were analyzed for SERPINE1, α-SMA and phosphorylated SMAD2 levels by Western blot and quantified densitometrically using ACTIN as loading control (FIG. 20D). * p<0.05, ** p<0.01

FIG. 21. Sirius Red staining on diaphragm muscles following systemic administration. Cross-section of diaphragm muscles were stained for connective tissue/collagen deposition by Sirius Red. Representative images were shown for each treatment group (FIG. 21A). The red stained area, representing the fibrotic part of the fibers, was quantified and expressed relative to the total fiber area using ImageJ. Four muscle sections were analyzed per animal and the average of six mdx/group was plotted (FIG. 21B). Error bars represent standard deviations **p<0.01.

FIG. 22. Serum protein analysis at the end point of systemic administration. Blood was taken from tails for each animal at the terminal time point and serum was collected. Urea, glutamic-pyruvic transaminase (GPT), glutamic-oxaloacetic transaminase (GOT) and creatine kinase (CK) levels were measured. The average values of all animals in each group was calculated and plotted. Error bars represent the standard deviation. Decrease in CK level showed indication of improved muscle quality, although this is not significant due to the variations between animals. Urea, GOT and GPT levels were not significantly increased, suggesting that this treatment is not toxic.

FIG. 23. Daily treatment of LY364947 did not alter dystrophic parameters in mdx mice. LY364947 were administered via intraperitoneal injections into five- to six-week-old mdx mice (n=6) at the dose of 1 mg/kg daily for six weeks. Serum was collected prior to sacrificing the animals for creatine kinase measurement. RNA was isolated from triceps and analyzed for fibrotic (α-SMA, Col1a1) and regeneration (Myog) markers by QPCR, using Gapdh as housekeeping gene.

### DETAILED DESCRIPTION

The disclosure is based, in part, on the surprising discovery that ACVR1B and TGFBR1 have non-redundant functions in the mesenchymal differentiation process. Myostatin can inhibit differentiation towards myogenic lineage (S. J. Lee (2004), Annu. Rev. Cell Dev. Biol. 20, 61-86). It was previously demonstrated that myostatin can signal through both ACVR1B and TGFBR1 (Rebbapragada et al. (2003), Mol. and Cell. Biol. 23:7230-7242) and both ACVR1B and TGFBR1 are expressed in myoblast cells (Y. Furutani et al. (2009), Cell Biochem. Funct. 27(8):578-82).

Therefore, it would be expected that the ability of myostatin to inhibit differentiation toward myogenic lineage would not be significantly affected by the inhibition of ACVR1B, since TGFBR1 should function redundantly and compensate for any loss of ACVR1B activity. Surprisingly, inhibition of ACVR1B does reduce myostatin signaling in cells of the myogenic lineage, whereas inhibition of TGFBR1 does not, suggesting that ACVR1B is preferentially utilized in these cells to transmit the myostatin signal. By contrast, TGFBR1 is preferentially used as the preferred myostatin type I receptor in other cell types. Contrary to the previous assumption that ACVR1B and TGFBR1 function redundantly, inhibition of ACVR1B or TGFBR1 results in differing phenotypes depending on which type I receptor is inhibited, which TGF-beta family ligand is present, and in which cell the inhibition occurs.

Inhibition of ACVR1B in myogenic cells inhibits myostatin signaling in these cells enhancing myoblast differentiation (Example 11), whereas inhibition of TGFBR1 in the same cells does not affect myostatin signaling. Inhibition of TGFBR1 in myogenic cells also enhances myoblast differentiation, presumably by inhibiting TGF-beta signaling in these cells.

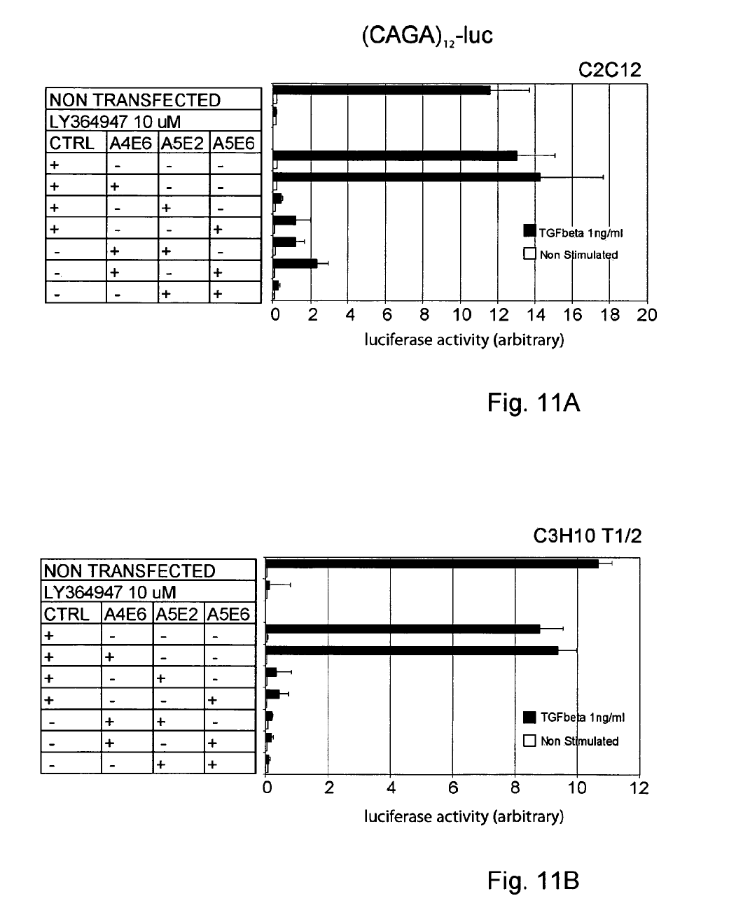

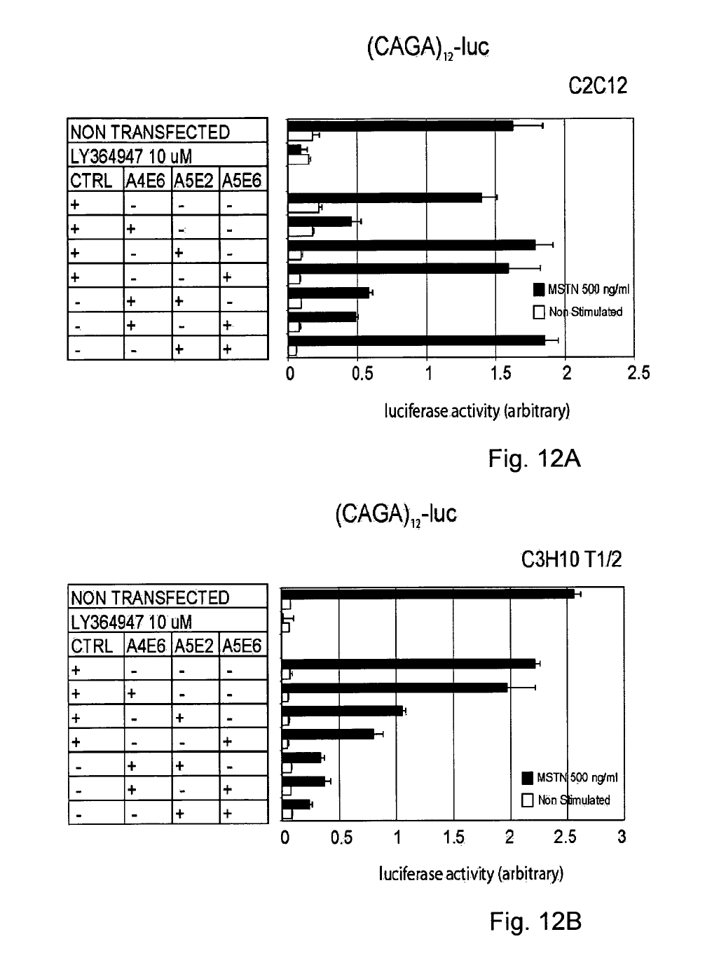

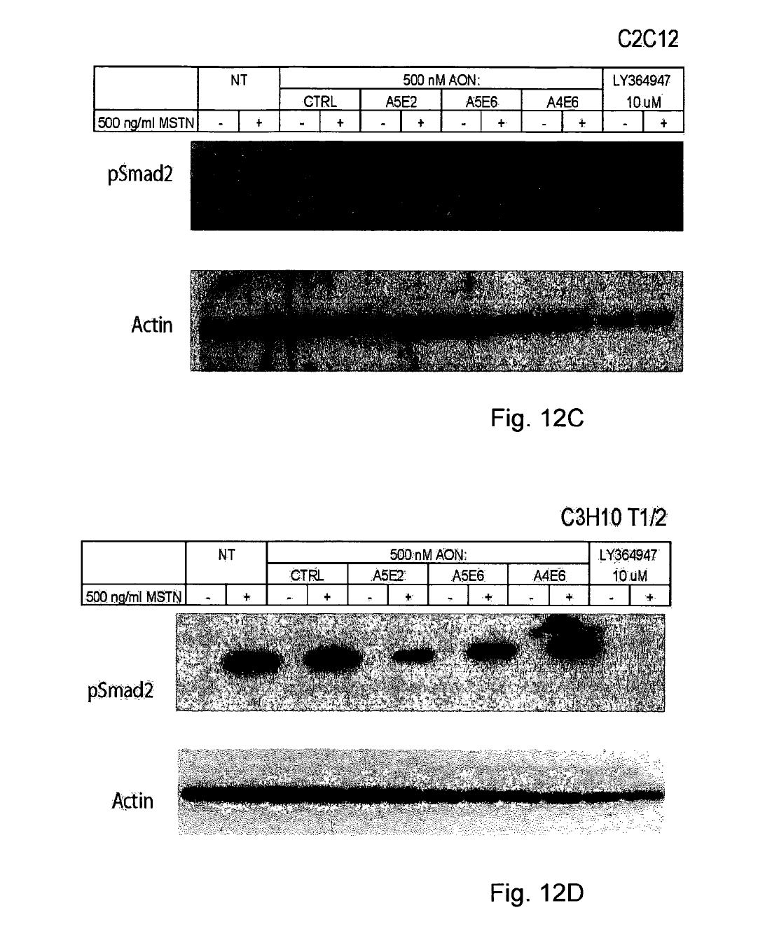

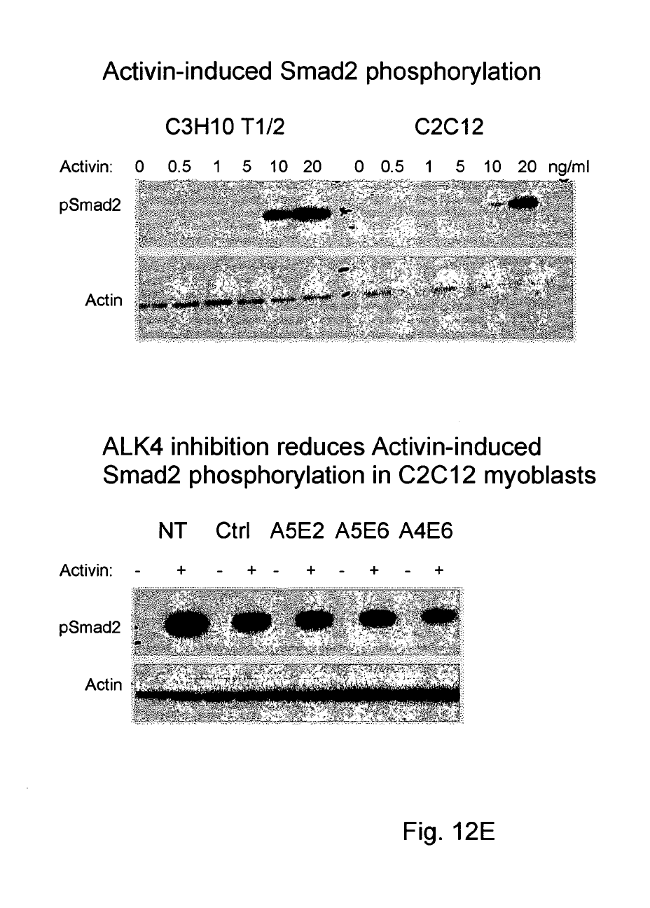

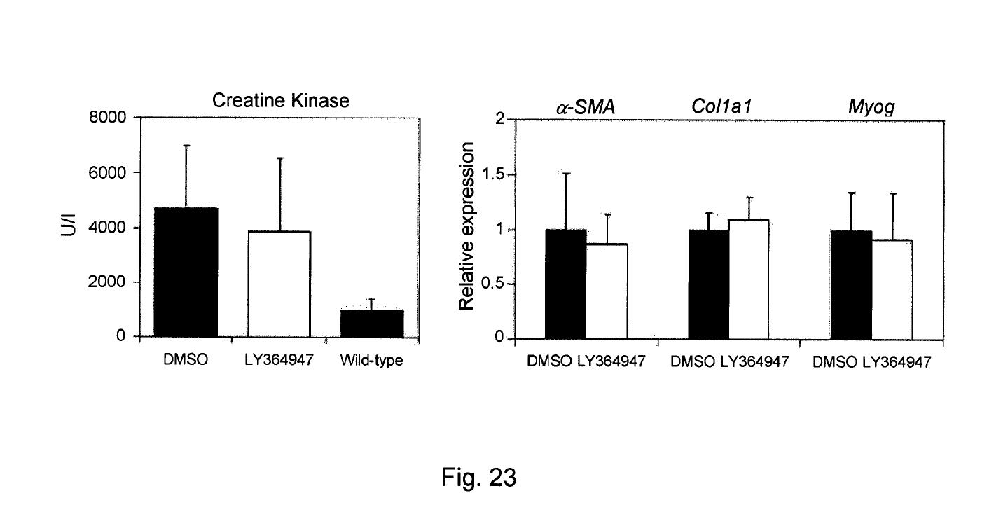

The present disclosure is also based in part on the finding that exon-skipping ACVR1B and TGFBR1 oligonucleotides can specifically inhibit ACVR1B and TGFBR1, in contrast to non-selective inhibitors known in the art, e.g., LY364947 (see FIGS. 11 and 12). Exon-skipping ACVR1B and TGFBR1 oligonucleotides were also demonstrated to be effective in vivo, in contrast to LY364947, which failed to induce any significant effects (see FIG. 23). Exon-skipping oligonucleotides offer several advantages over, for example, RNA interference molecules including the ability to more precisely titrate the inhibitory effects and the ability to target the oligonucleotides safely and effectively to the cells of interest. These and other advantages are further discussed herein.

As used herein, ACVR1B refers to activin receptor type-1B, also known as activin receptor-like kinase 4 (ALK-4). The human gene sequence encoding an exemplary ACVR1B has NCBI Reference No. NG_{—}022926.1.

As used herein, TGFBR1 refers to transforming growth factor, beta receptor I, also known as activin receptor-like kinase 5 (ALK-5). The human gene sequence encoding an exemplary TGFBR1 has NCBI Reference No. NG_{—}007461.1.

In one aspect, the disclosure provides a method for promoting muscle growth or treating muscle loss or insufficient muscle growth in an animal, comprising administering to the animal an effective amount of a compound that specifically reduces the amount or activity of ACVR1B protein and/or a compound that specifically reduces the amount or activity of TGFBR1 protein in a cell of the animal. In a preferred embodiment, a compound that specifically reduces the amount or activity of ACVR1B protein is administered and a compound that specifically reduces the amount or activity of TGFBR1 protein is not administered to the animal. In a preferred embodiment, a compound that specifically reduces the amount or activity of TGFBR1 protein is administered and a compound that specifically reduces the amount or activity of ACVR1B protein is not administered to the animal.

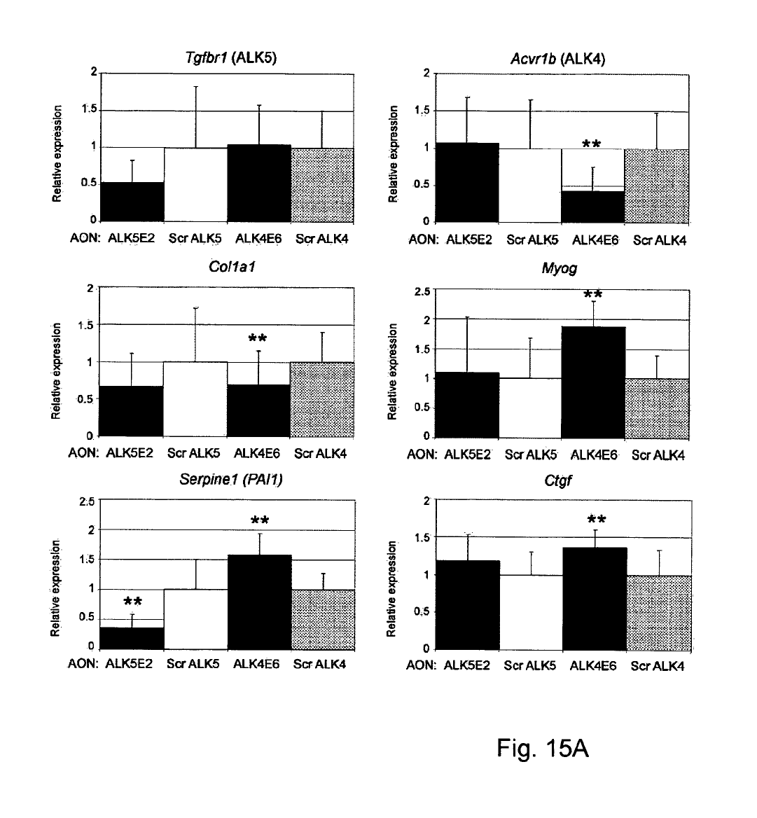

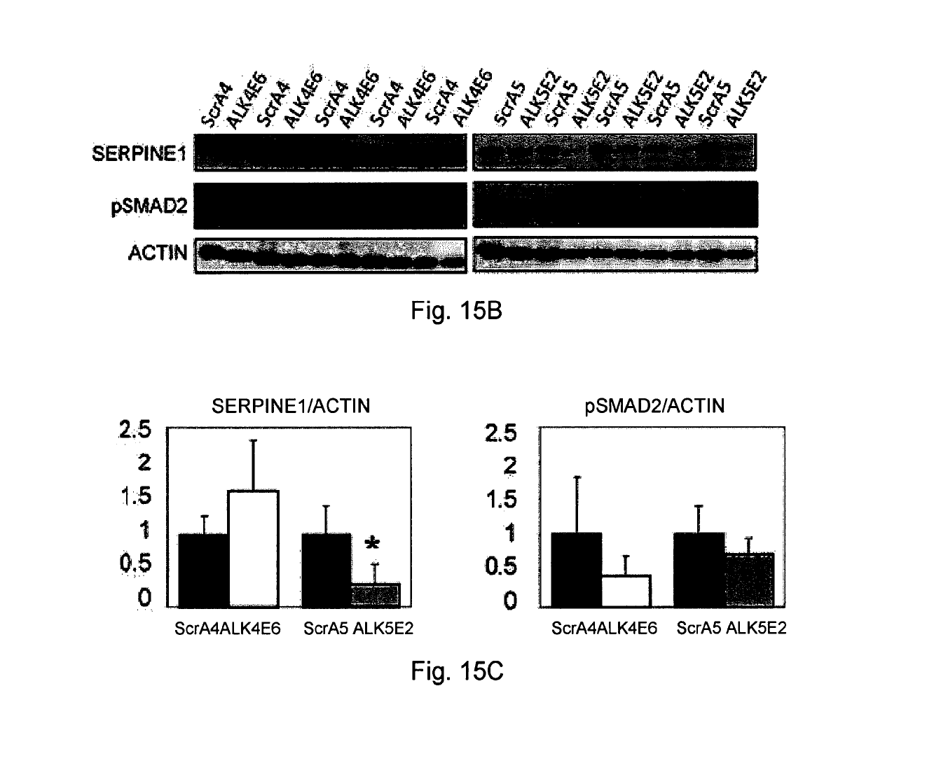

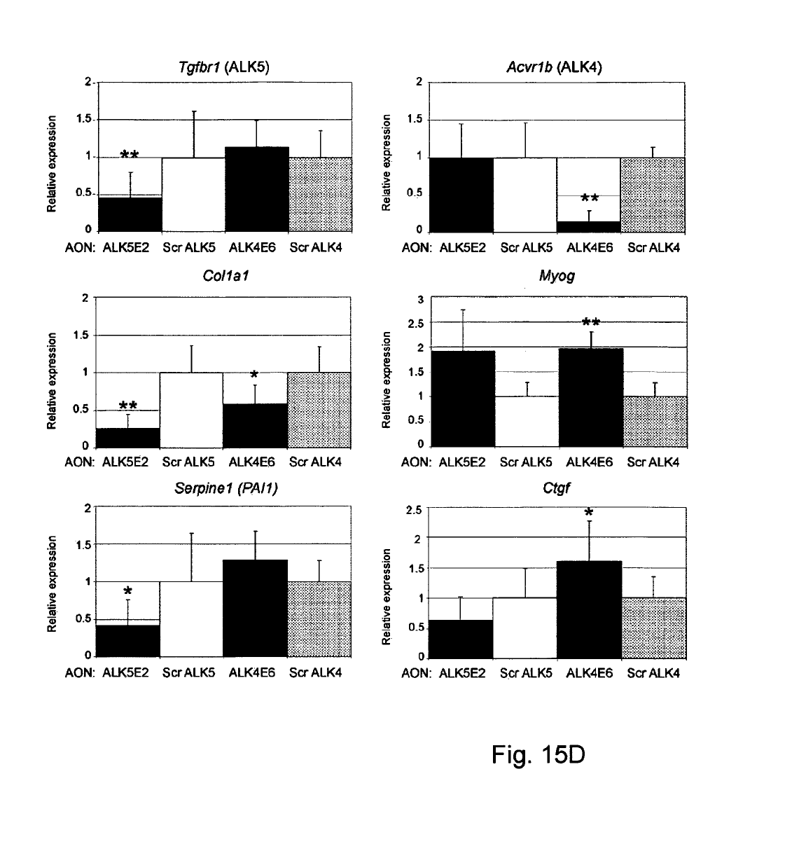

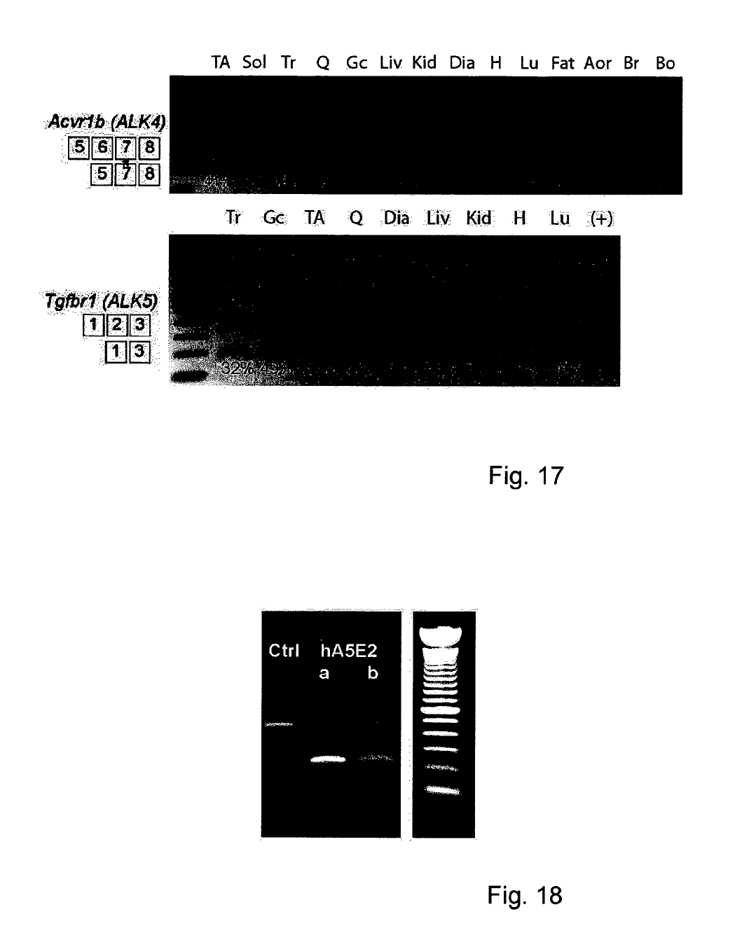

ACVR1B is expressed in various tissues including brain, lung, heart, liver, and kidney (see, e.g., K. Verschueren et al. (1995), Mech. Dev. 52:109-123), while TGFBR1 is also expressed in various tissues including lungs, muscle, and olfactory epithelium (T. Seki et al. (2206), Lab. Invest. 86(2):116-29). It would be expected that reducing the expression or activity of either ACVR1B or TGFBR1 in cells of an animal would result in numerous deleterious effects. To the contrary, it was surprisingly found that exon-skipping oligonucleotides directed toward ACVR1B and TGFBR1 result in a reduction in the respective protein levels with a surprising lack of toxicity (FIGS. 15-17, 22).

Therapeutics have been developed based on soluble ACVR2B receptors that can bind myostatin (ACE-031 from Acceleron), thereby acting as an inhibitor of myostatin signaling. Such compounds have been reported to have side effects such as nose bleeds. The present invention provides compounds that do not directly inhibit ligands from the TGF-beta family (myostatin, activin, TGF-beta), but rather inhibit the function of a specific type I receptor (ACVR1B or TGFBR1).

The methods and compositions provided herein may be used to treat muscle loss or insufficient muscle growth resulting from any disease, disorder, or condition. The methods and compositions provided herein may also be used to treat or alleviate a symptom of any disorder, disease, or condition, wherein the symptom is muscle loss or insufficient muscle growth.

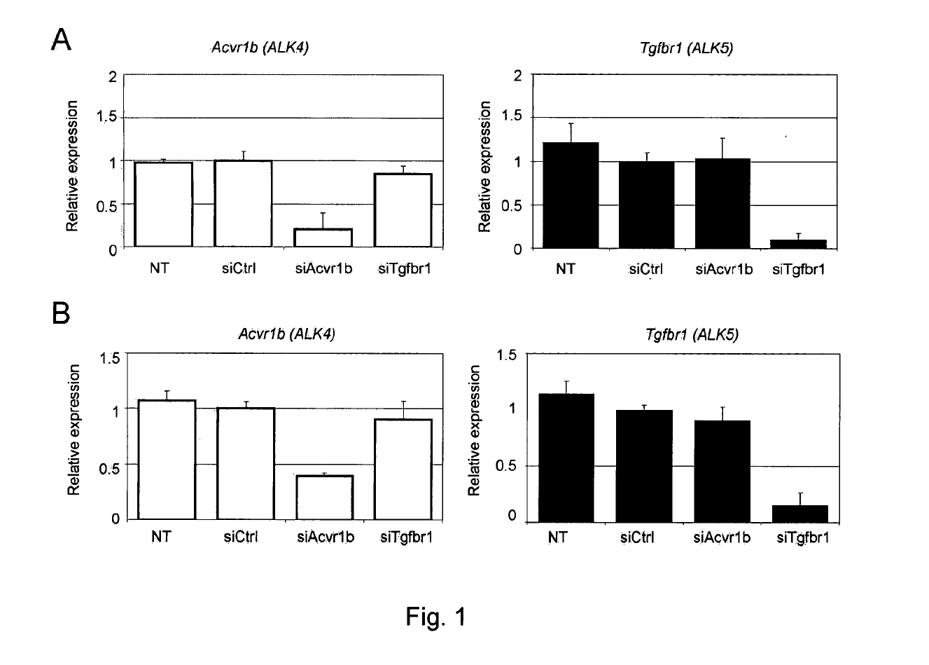

Muscle loss and insufficient muscle growth may be the result of e.g., abnormal differentiation of muscle precursor cells, malfunctioning of differentiated muscle cells, or damage/destruction of muscle precursor cells, differentiated muscle cells, or muscle fibers or a microenvironment in the muscle that is not supportive for muscle differentiation, e.g., due to the presence of fibrotic tissue or inflammatory cells that secrete signaling molecules, such as ligands of the TGF-beta family that inhibit muscle differentiation. Enhanced muscle differentiation can thus be achieved by targeting the primarily involved myogenic cells but also by targeting the secondarily involved non-muscle cells. Accordingly, the disclosure provides methods and compounds to specifically target activin receptor-like kinase signaling in myogenic cells as well as methods to specifically target activin receptor-like kinase signaling in non-myogenic cells. These methods and compounds inhibit signaling of ligands from the TGF-beta family implicated in muscle differentiation, preferably myostatin, activin and TGF-beta (FIGS. 1-4, 6, 7, 10, 11, 12, 14, 16, 19, and 20).

Muscle loss or insufficient muscle growth are symptoms of any number of disorders, including, “skeletal muscle degenerative diseases” or “skeletal muscle wasting diseases.” In these diseases, degeneration (atrophy) occurs in skeletal muscle cells or structural changes or functional impairment occur in skeletal muscle. Skeletal muscle atrophy can occur with chronic pulmonary disease (COPD), pathologies associated with a reduced glycolytic rate such as McArdle's disease and phosphofructokinase disease (PFKD), as a result of muscle denervation (e.g., amyotrophic lateral sclerosis, Guillain-Barre syndrome and diabetic neuropathy), spinal muscular atrophies, (e.g., SMA, SMA1, SMA2, SMA3, SBMA), traumatic injury, immobility-induced muscle loss, or chemotherapy. Muscle loss is also a symptom of cachexia (e.g., which frequently occurs in cancer patients), aging-related sarcopenia, inflammatory myopathies (e.g., Dermatomyositis, Polymyositis, and Inclusion Body Myositis). In preferred embodiments, the muscle loss or insufficient muscle growth are symptoms of a muscular dystrophy (e.g., Congenital Muscular Dystrophy (CMD), Distal Muscular Dystrophy (DD), Emery-Dreifuss Muscular Dystrophy (EDMD), Facioscapulohumeral Muscular Dystrophy (FSH), Limb-Girdle Muscular Dystrophy (LGMD), Myotonic Dystrophy (MMD), Oculopharyngeal Muscular Dystrophy (OPMD), preferably, Becker Muscular Dystrophy (BMD) and/or Duchenne Muscular Dystrophy (DMD)). The promotion of muscle growth may be used in disorders of muscle loss or of insufficient muscle growth, e.g., as a symptom of muscular dystrophy.

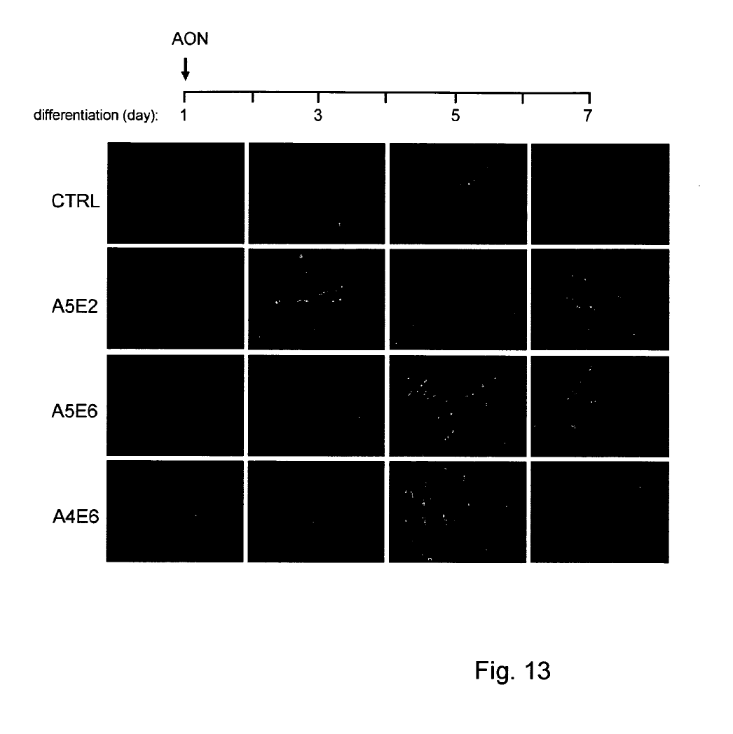

As used herein, “treating muscle loss or insufficient muscle growth” refers to the prevention or reduction of muscle loss in comparison to non-treatment, as well as promoting the differentiation of muscle precursor cells and promoting muscle fiber formation (FIGS. 13 15, 19 and 20). The treatment includes alleviating one or more muscle-related symptoms of a disorder including, e.g., muscle atrophy, formation of fibrotic tissue, adipose tissue or calcified lesions, leading to reduced muscle strength.

Alleviating one or more symptom(s) of a disorder associated with muscle loss or insufficient muscle growth in an individual using a method or compound disclosed herein may be assessed by any of the following assays: prolongation of time to loss of walking, improvement of muscle strength, improvement of the ability to lift weight, improvement of the time taken to rise from the floor, improvement in the six-minute walk test, the nine-meter walking time, improvement in the time taken for climbing four stairs, improvement of the leg function grade, improvement of the pulmonary function, improvement of cardiac function, improvement of the quality of life. Each of these assays is known to the skilled person.

The alleviation of one or more symptom(s) may also be assessed by measuring an improvement of a muscle fiber function, integrity and/or survival. Such assays include: a detectable decrease of creatine kinase in blood, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

In a preferred embodiment of the methods described herein, administration of a compound that specifically reduces the amount or activity of ACVR1B protein in a cell of the animal reduces myostatin signaling or the ability to transduce myostatin signaling in the cell; preferably, the cell is a cell of the myogenic lineage. Myostatin, also known as growth differentiation factor 8 (GDF8), is encoded in humans by the MSTN gene (N. F. Gonzalez-Cadavid et al. (1998), Proc. Natl. Acad. Sci. U.S.A. 95 (25):14938-43). In a preferred embodiment, administration of a compound that specifically reduces the amount or activity of TGFBR1 protein in a cell of the animal reduces myostatin signaling or the ability to transduce myostatin signaling in the cell; preferably, the cell is a cell that is not of the myogenic lineage. In a preferred embodiment, administration of a compound that specifically reduces the amount or activity of TGFBR1 protein in a cell of the animal reduces TGF-beta signaling or the ability to transduce TGF-beta signaling in the cell. As discussed above, the muscle microenvironment plays a role in supporting muscle growth and differentiation. Therefore, influencing TGF-beta family signaling in cells not of the myogenic lineage also provides a means to treat muscle loss or insufficient muscle growth.

As used herein, a cell of the myogenic lineage includes satellite cells (skeletal muscle progenitor cells responsible for post-natal growth and repair), myoblasts (i.e., muscle progenitor cells), muscle stem cells, muscle cells, and muscle fibers. Proliferating myoblasts express MYOD1 and MYF5, and upon initiation of myogenic differentiation expression of MYOG and MRF4. The presence of any of these proteins may be used as markers of cells of the myogenic lineage. Preferably, markers of cells of the myogenic lineage include MYOD1 and Pax3/7.

Although not wishing to be bound by theory, effective myostatin signaling in cells of the myogenic lineage may depend on the co-expression of ACVR1B and a specific co-receptor. The in vitro experiments provided in the Examples provide TDGF1, an EGF-CFC family member, as one candidate for an ACVR1B co-receptor in myogenic cells. ACVR1B also acts as a receptor for Nodal and Activin signaling. However, while Nodal requires EGF-CFC co-receptors for signaling, Activin signaling does not (see, e.g., Cheng et al. (2003), Genes Dev. 17:31-36). It was also recently reported that TDGF1 inhibits myostatin signaling (Ciarmela et al. (2011), J. Clin. Endocrinol. Metab. 96:755-765). These experiments were performed in 293T cells transfected with TDGF1 and a Smad2-responsive luciferase reporter, however, it is not clear which receptors were responsible for transmitting the myostatin signal in these cells. The decrease in myostatin signaling observed in Ciarmela et al. is restricted to the effects of TDGF1 in a context where TDGF1 cannot bind to ACVR1B because the expression level of ACVR1B in these cells is too low. Moreover, in Table 1 of Ciarmela et al., the accession number for TDGF1 is listed as NM_{—}032545. This accession number actually refers to CFC1, a different member of the EGF-CFC family. Thus, the decrease in myostatin signaling observed in Ciarmela et al. is due to expression of CFC1.

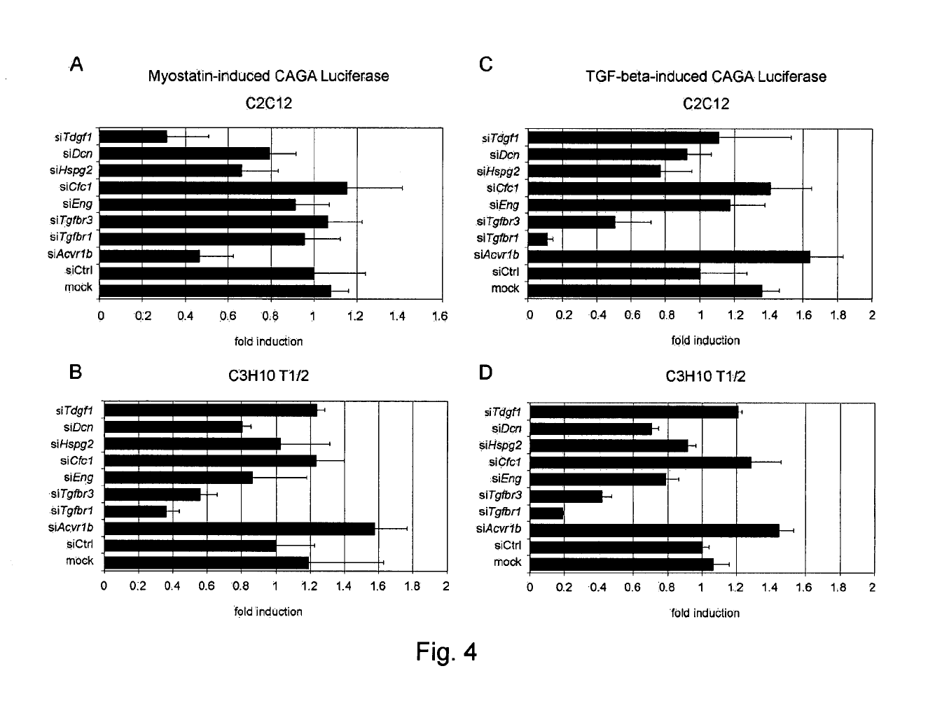

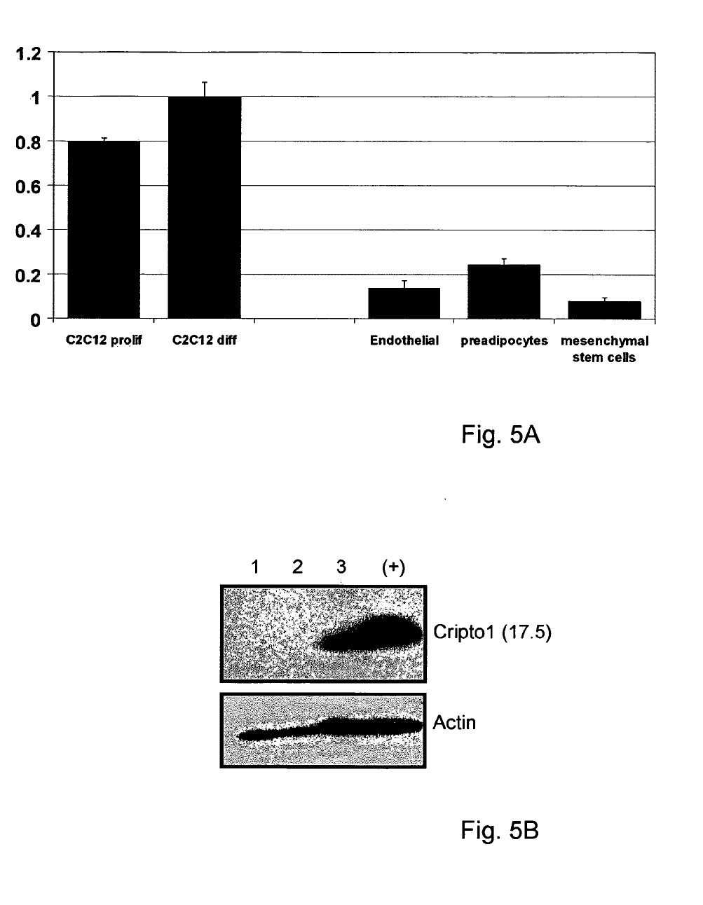

It was found that TDGF1 is expressed in cells, such as myoblast cells, which preferentially utilize ACVR1B as the myostatin type 1 receptor over TGFBR1, but is not significantly expressed in cells, such as mesenchymal stem cells and pre-adipocytes, which preferentially utilize TGFBR1 as the myostatin type 1 receptor over ACVR1B (FIG. 5). A reduction in TDGF1 expression in myoblast cells results in a reduction of ACVR1B-mediated myostatin activity (FIG. 4, Panel A). In addition, evidence that TDGF1 is expressed in satellite cells isolated from adult muscle fibers was also obtained. The presence or absence of TDGF1, or a similar co-receptor, is one mechanism by which a cell that expresses both ACVR1B and TGFBR1 can transmit the myostatin signal via a “preferred” type 1 receptor.

Although ACVR1B and TGFBR1 are both expressed in cells of the myogenic lineage and would thus be expected to function redundantly in transmitting myostatin signaling, it was found that cells of the myogenic lineage preferentially utilize ACVR1B as the myostatin type 1 receptor. Accordingly, the disclosure provides methods for stimulating differentiation of a cell in the myogenic lineage comprising providing the cell with a compound that specifically reduces the amount or activity of ACVR1B protein in the cell, preferably, wherein myostatin signaling is silenced. Preferably, the method is an in vitro method, and preferably myostatin is also provided.

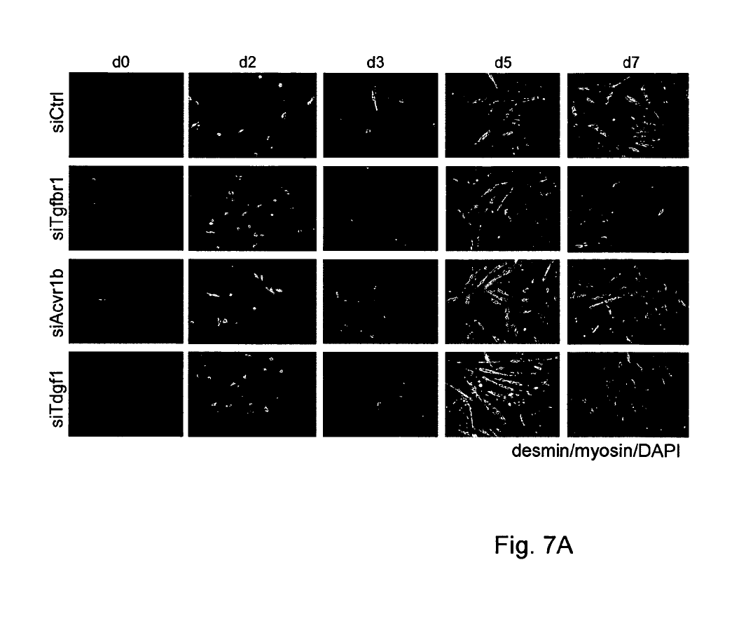

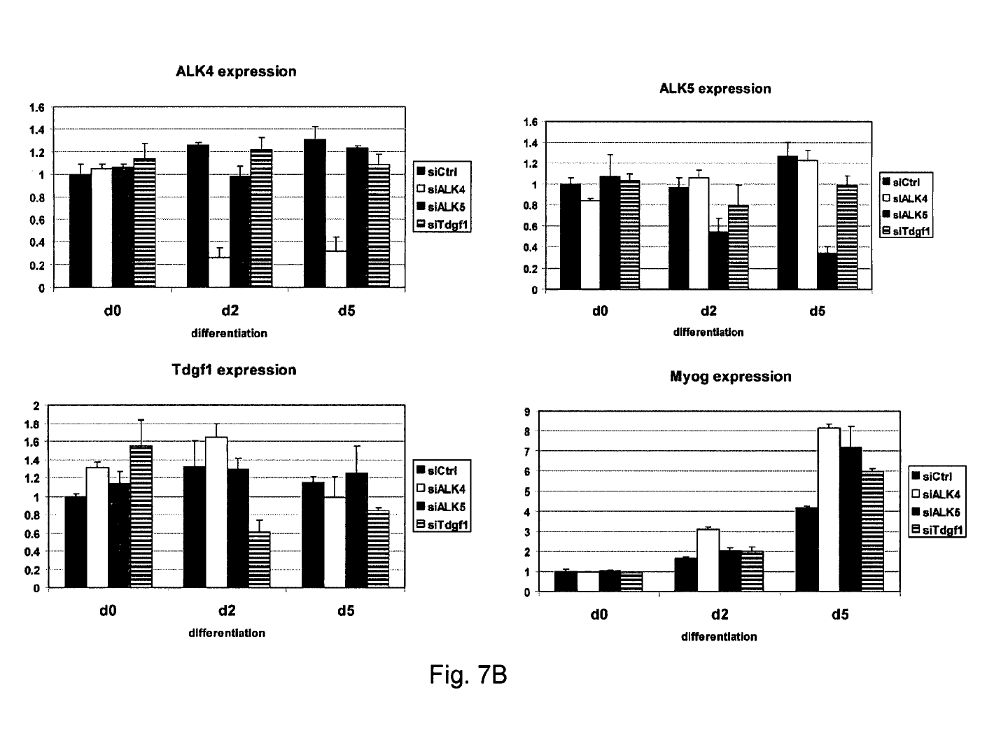

As shown in FIGS. 7 and 13, providing a compound that specifically reduces the amount or activity of ACVR1B stimulates differentiation in myoblast cells. The TGFBR1 expressed in these cells is unable to compensate for a loss of ACVR1B activity, suggesting that myostatin signaling (via ACVR1B) is required for inhibiting differentiation. In contrast, cells not belonging to the myogenic lineage, e.g., C3H10T1/2, 3T3-L1 and endothelial cells, preferentially utilize TGFBR1 to transmit the myostatin signal.

Although cells of the myogenic lineage preferentially utilize ACVR1B as the myostatin type 1 receptor, it was found that TGFBR1 is also involved in differentiation of cells in the myogenic lineage (FIGS. 7 and 13). While not wishing to be bound by theory, these effects of TGFBR1 are likely due to its function as a TGF-beta receptor. Accordingly, the disclosure further provides a method for stimulating differentiation of a cell in the myogenic lineage comprising providing the cell with a compound that specifically reduces the amount or activity of TGFBR1 protein in the cell, preferably, wherein TGF-beta signaling is reduced.

Stimulating differentiation of a cell in the myogenic lineage includes inducing changes in cell morphology from diamond-shaped, desmin-positive myoblast cells to spindle-like cells, with subsequent fusion between myoblasts to form multinucleated myotubes. These myotubes are positive for desmin, myosin, and other sarcomeric proteins.

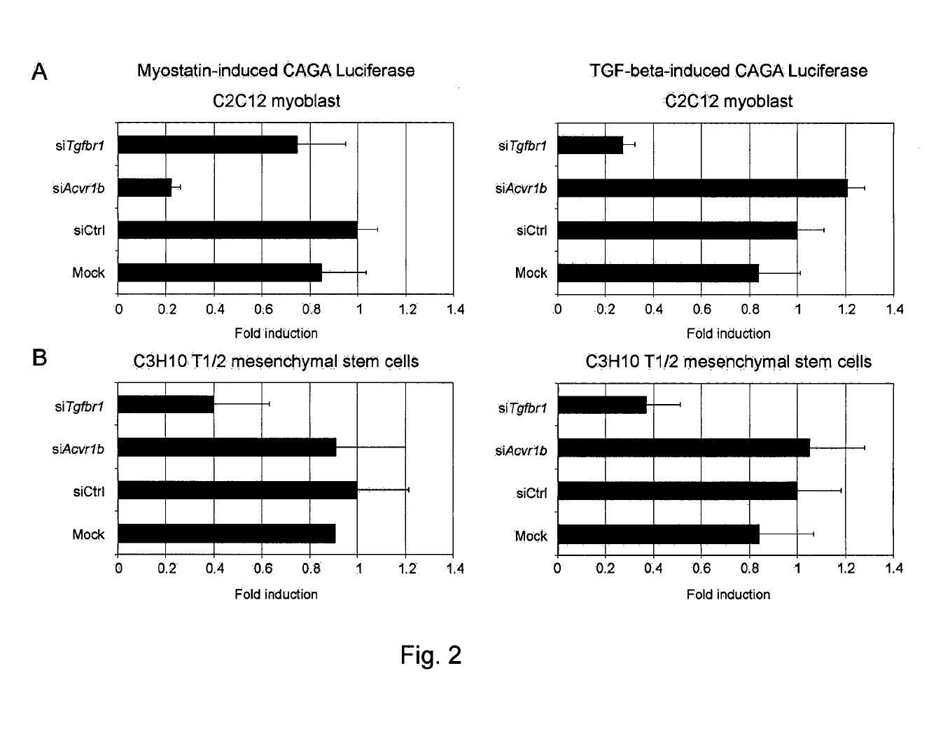

It has recently been reported that TDGF1 competes with TGFBR1 for binding to TGF-beta, thereby blocking TGF-beta signaling (see, e.g., US 2005/0208045). Reducing the amount or activity of TGFBR1 in TDGF1-expressing cells would thus not be expected to significantly reduce TGF-beta signaling. To the contrary, it was found that TGFBR1 is able to transmit TGF-beta signaling in TDGF1-expressing cells and that by reducing TGFBR1 activity in these cells, TGF-beta signaling is reduced (FIG. 2, Panel A). Accordingly, the disclosure provides a method for silencing TGF-beta signaling in a cell that is permissive to TGF-beta signaling comprising selecting a cell that expresses TGFBR1 and a member of the EGF-CFC gene family, preferably TDGF1, and silencing TGFBR1 in the cell.

Also provided is a method for silencing myostatin and/or activin signaling in a cell that is permissive to myostatin and/or activin signaling comprising selecting a cell that expresses a) ACVR2A and/or ACVR2B, b) ACVR1B, and c) TGFBR1 and providing to the cell an effective amount of a compound that specifically reduces the amount or activity of ACVR1B protein and/or a compound that specifically reduces the amount or activity of TGFBR1 protein in the cell. Preferably, the cell is a cell in the myogenic lineage and a compound that specifically reduces the amount or activity of ACVR1B protein is provided. Preferably, the cell is a cell not in the myogenic lineage and a compound that specifically reduces the amount or activity of TGFBR1 protein is provided. Preferably, the method is an in vitro method.

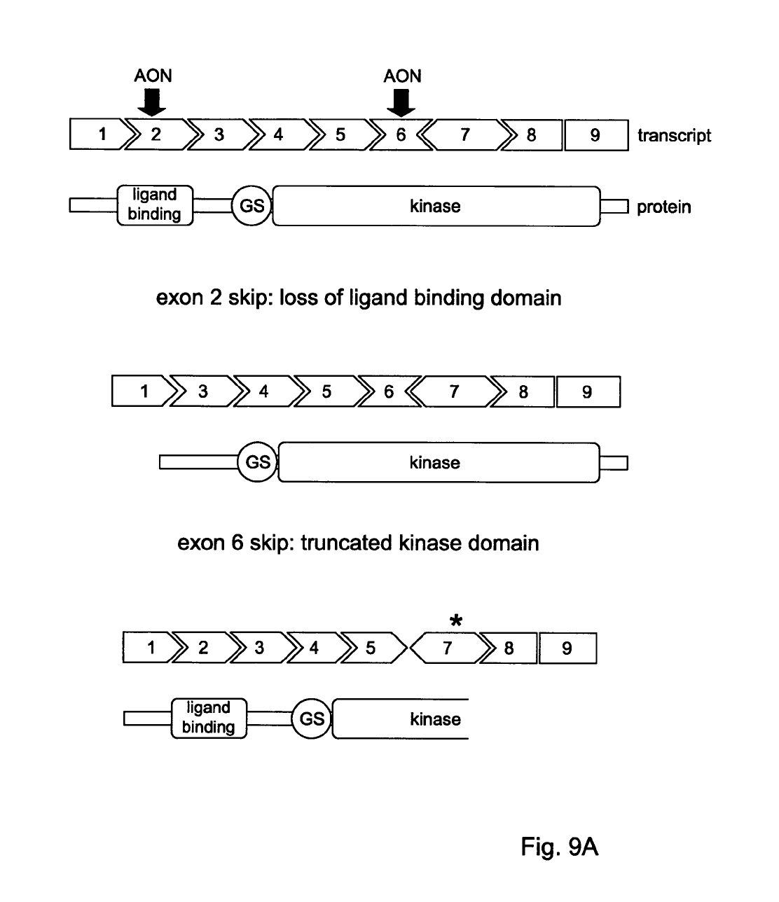

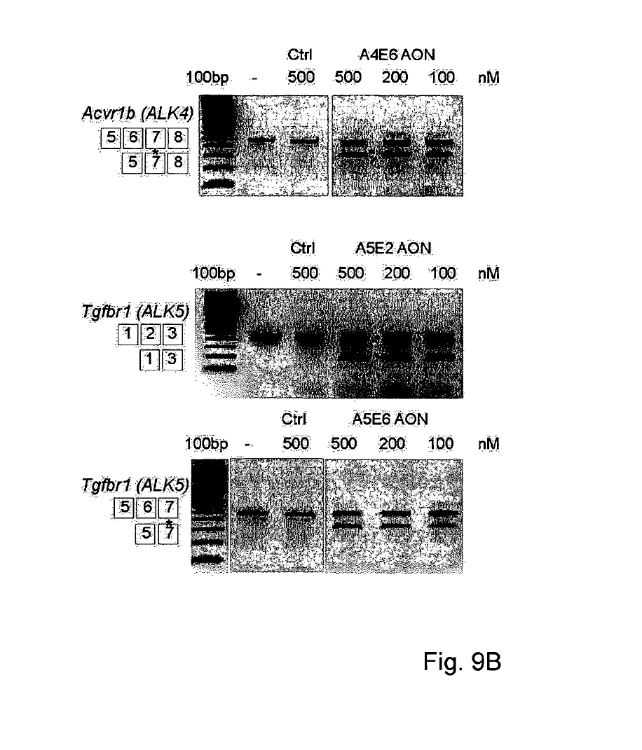

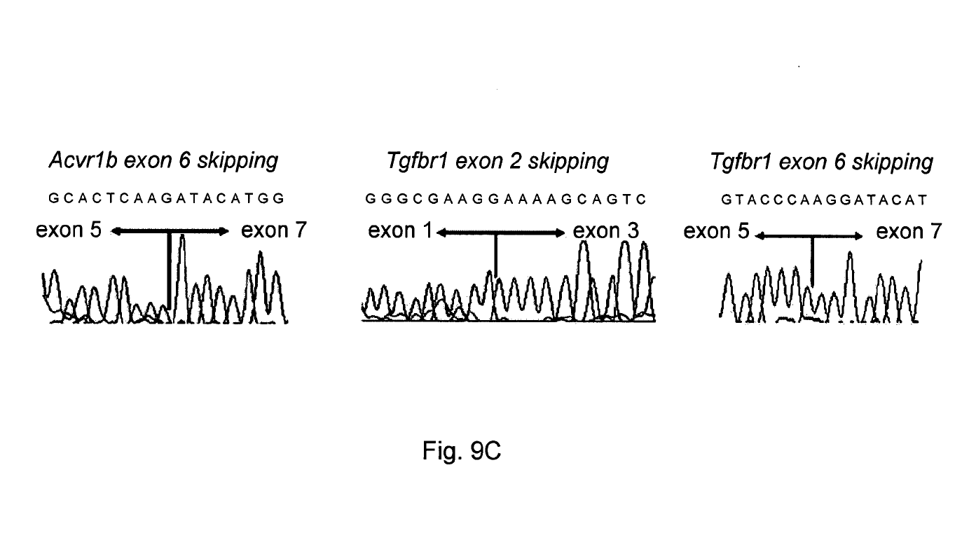

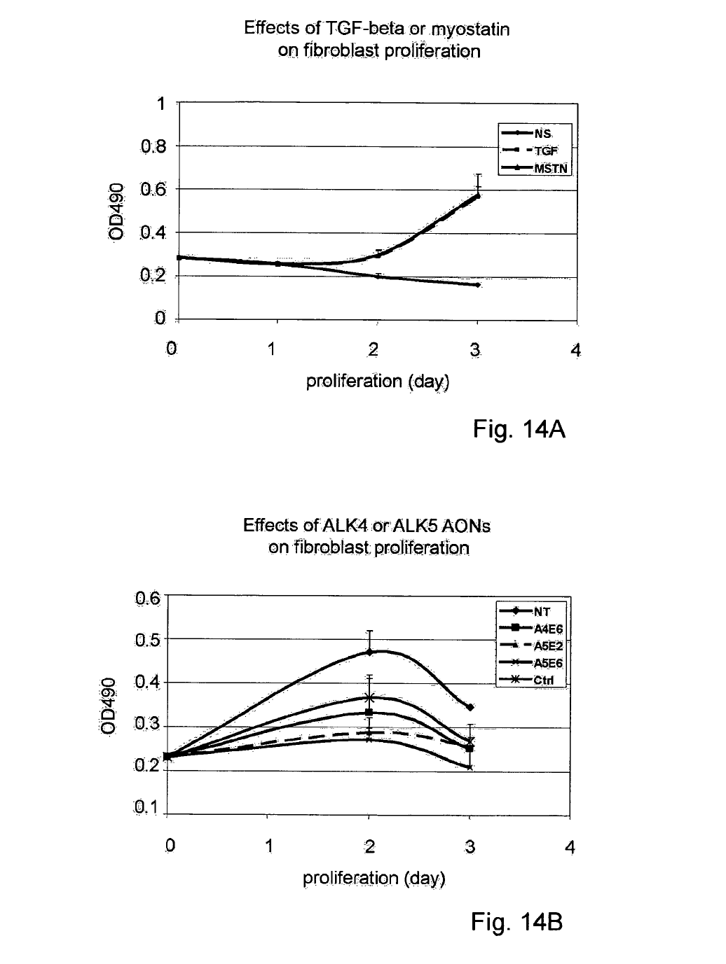

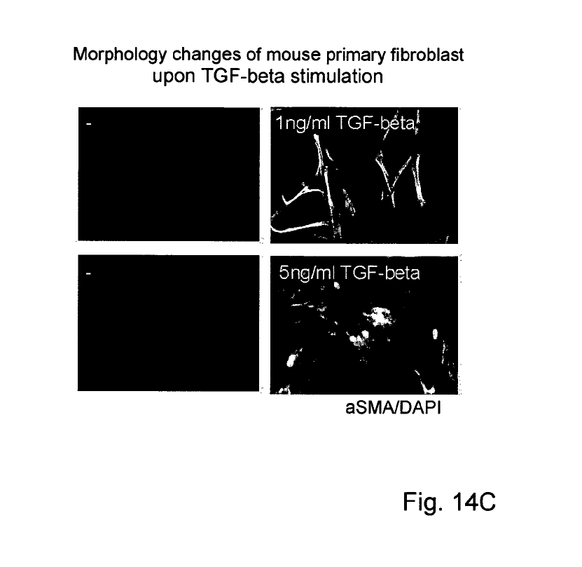

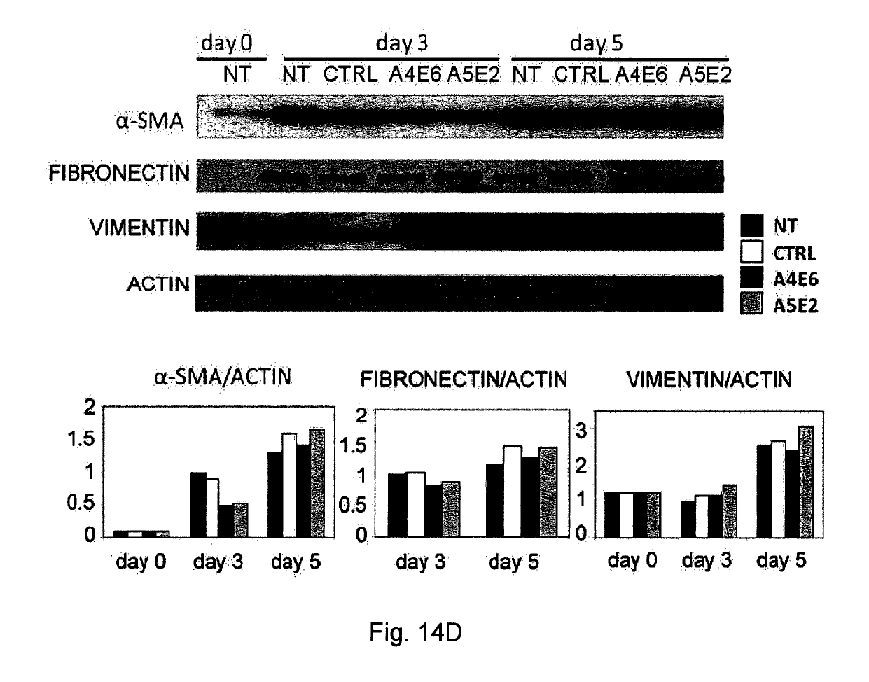

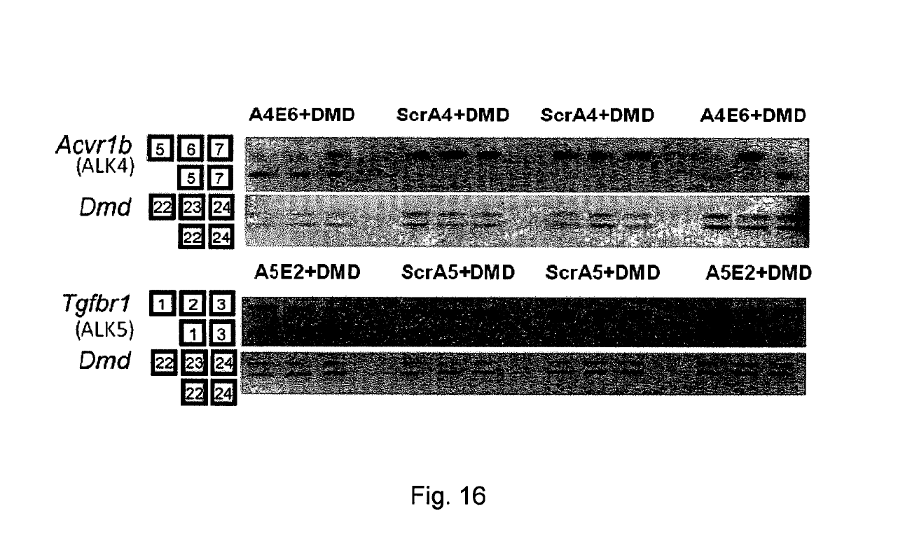

The disclosure is also based, in part, on the finding that specific inhibition of TGFBR1 can decrease fibroblast proliferation (FIG. 14B) and fibrosis (as measured by the expression of the fibrotic markers SERPINE1, FIGS. 15 and 16 and alpha-SMA, FIGS. 14, 19, and 20). The current small molecule TGFBR1 inhibitors currently under investigation, such as LY364947, LY550410, LY580276, and SB505124 inhibit not only TGFBR1, but other proteins such as ACVR1B and ACVR1C (Yingling et al. (2004), Nature Rev. Drug Discovery 3(12):1011-22). It is, therefore, not clear which receptor is responsible for the effects observed by the use of such compounds and administration of such compounds could lead to additional effects not associated with TGFBR1 inhibition. Accordingly, the disclosure provides compounds that specifically reduce the amount or activity of TGFBR1 protein without affecting ACVR1B or ACVR1C (FIGS. 9, 10, 12, 15, 16, 19, 20). These compounds can, therefore, be used to treat fibrosis and decrease the proliferation or differentiation of fibroblast cells without producing any negative side effects associated with ACVR1B or ACVR1C inhibition.

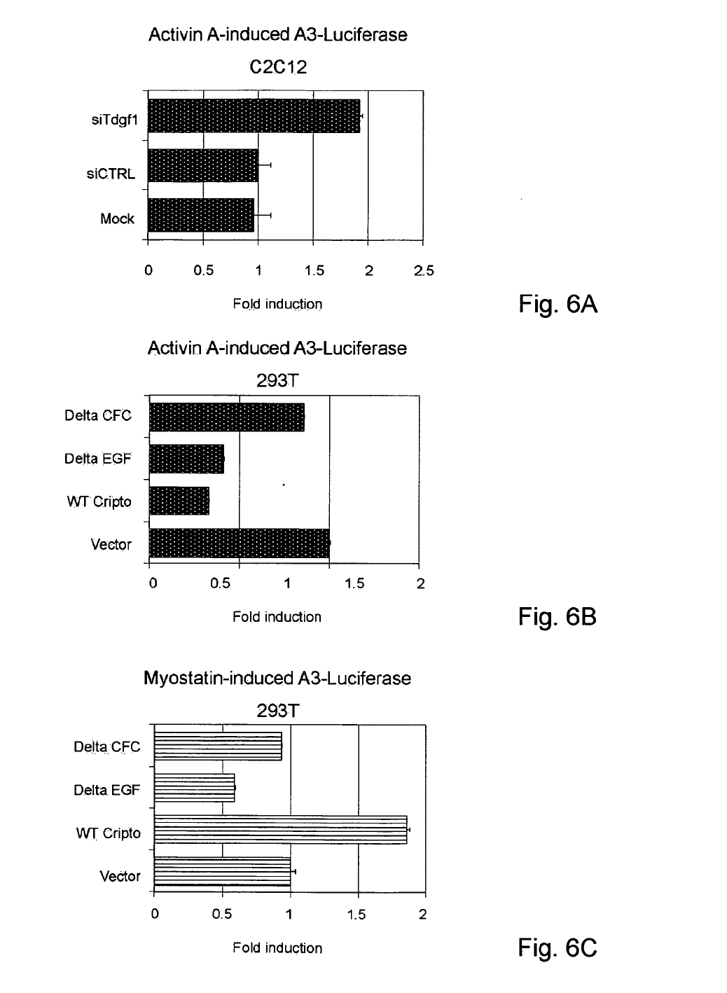

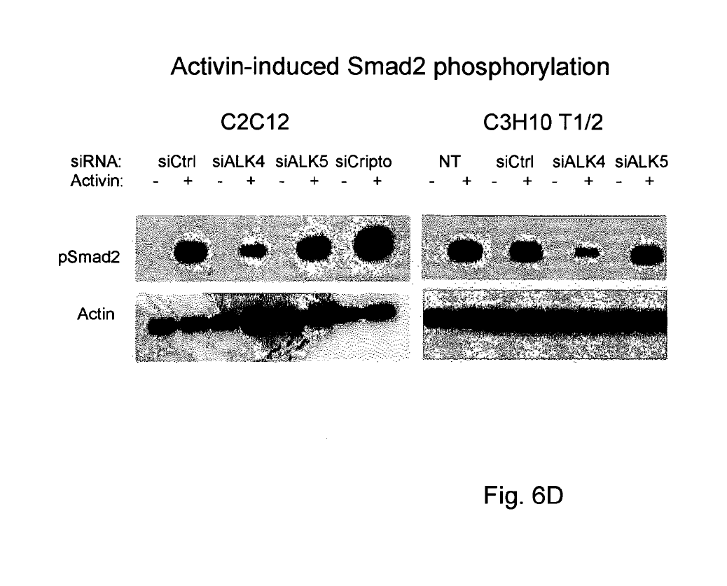

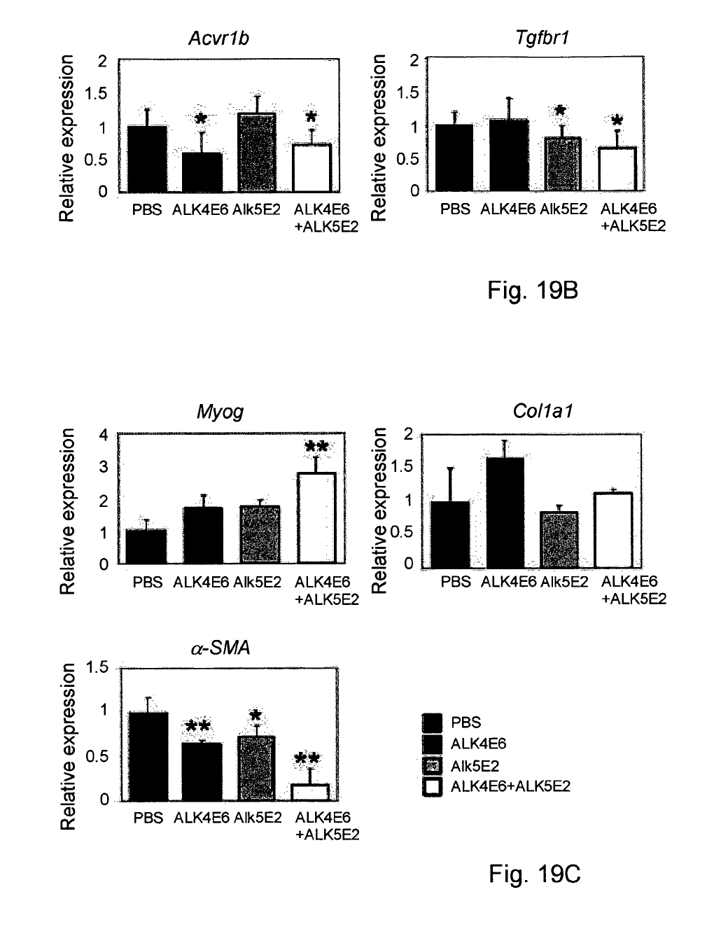

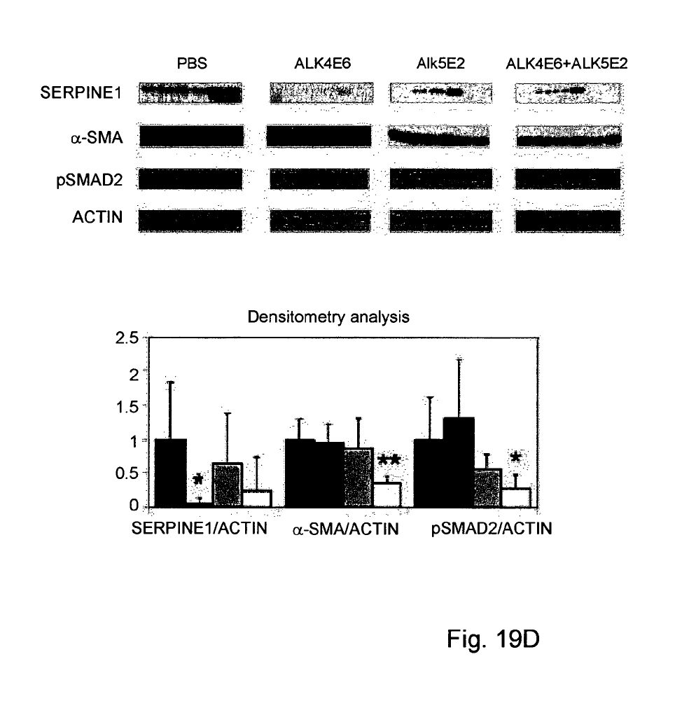

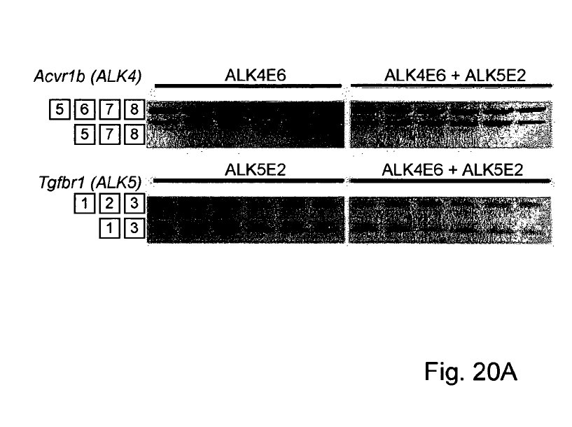

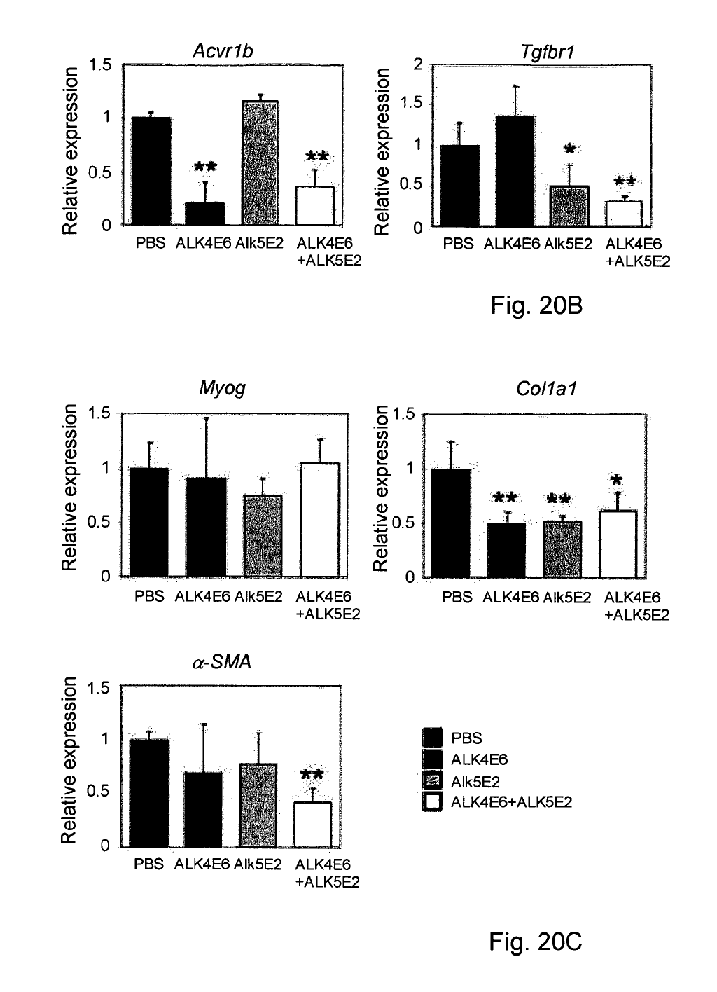

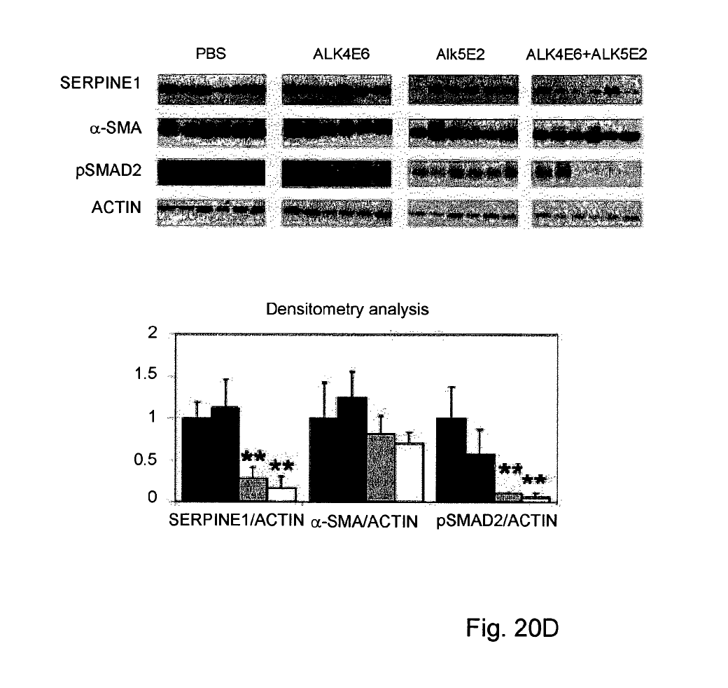

The disclosure is also based, in part, on the finding that specific inhibition of ACVR1B can decrease the differentiation of fibroblasts in vitro (FIG. 14D) and decrease fibrosis in vivo, as measured by the fibrosis marker alpha-SMA (FIGS. 19 and 20) or collagen deposition (FIG. 21). This finding was unexpected since it was also demonstrated that TGF-beta and myostatin, the main fibrotic agents in muscle, signal exclusively via TGFBR1 and not through ACVR1B in fibroblasts (see, e.g., FIGS. 2 and 4). Likewise, it was unexpected to find additive effects of the knock-down of ACVR1B and TGFBR1 in mdx mice on the expression of myofibroblast marker alpha-smooth muscle actin (FIG. 19C), suggesting a role for AVCR1B in fibrotic lesion formation in the muscle. This may potentially be through activin signaling, which is ACVR1B-dependent in both myoblasts and fibroblasts (FIGS. 6D and 12E). These findings also support the suggestion that the effects of inhibiting different components of the TGF-beta superfamily are difficult to predict.

The methods and compounds described herein are useful for decreasing or preventing fibrosis associated with wounds, hepatitis B or C infection, Schistosoma infection, kidney disease, heart disease, macular degeneration, retinal and vitreal retinopathy, systemic and local scleroderma, atherosclerosis, restenosis, chemotherapeutic drug-induced fibrosis, radiation-induced fibrosis, burns, as well as pulmonary fibrosis, hepatic fibrosis (cirrhosis), renal fibrosis, corneal fibrosis, heart fibrosis, osteoarticular fibrosis, tissue fibrosis, tumor stroma, desmoplastic tumors, surgical adhesions, hypertrophic scars, and keloids, and fibrosis affecting a tissue such as muscle tissue, skin epidermis, skin dermis, tendon, cartilage, pancreatic tissue, uterine tissue, neural tissue, testis, ovary, adrenal gland, artery, vein, colon, small and large intestine, biliary tract, and gut.

Fibrosis is characterized by the formation of fibrotic tissue or tissue damage. This involves a pathological accumulation of connective tissue cells in the connective tissue itself or in an organ. The tissue of the organ in question becomes hardened, thereby resulting in scar tissue changes, which then, in an advanced stage, lead to restriction of the respective organ function.

Muscle fibrosis is a phenomenon that frequently occurs in diseased or damaged muscle. It is characterized by the excessive growth of fibrous tissue, which usually results from the body's attempt to recover from injury. Fibrosis impairs muscle function and causes weakness. The amount of muscle function loss generally increases with the extent of fibrosis. Fibrosis commonly occurs as a result of muscular dystrophy, as well as due to other afflictions, such as denervation atrophy, a degradation of muscle tissue caused by loss of neural contact to a muscle.

In one aspect, provided is a method for inhibiting, decreasing and/or preventing the development of fibrosis in an animal comprising administering to the animal in need thereof an effective amount of a compound that specifically reduces the amount or activity of TGFBR1 protein in a cell of the animal, preferably, wherein the fibrosis is selected from muscle, pulmonary, liver, or cardiac fibrosis, more preferably, wherein the fibrosis is selected from muscle fibrosis. Preferably, the compound is an exon-skipping oligonucleotide.

The effect of TGFBR1 inhibition on fibroblast proliferation provides a useful treatment for fibrosis, which is not limited to any particular tissue. Since fibroblast proliferation and differentiation are essential features of fibrosis, the reduction in fibroblast proliferation and delayed fibroblast differentiation by the compounds disclosed herein results in the decrease or prevention of fibrosis development generally. Preferably, TGFBR1 inhibition is useful for treating a fibrosis associated with increased TGF-beta signaling. Preferably, TGFBR1 inhibition is useful for treating muscle, pancreatic, kidney, and pulmonary fibrosis.

In one aspect, described is a method for inhibiting, decreasing and/or preventing the development of fibrosis in an animal comprising administering to the animal in need thereof an effective amount of a compound that specifically reduces the amount or activity of ACVR1B protein in a cell of the animal. Preferably, ACVR1B inhibition is useful in a fibrosis associated with activin overexpression. Preferably, the fibrosis is selected from skin, pancreatic, muscle, pulmonary, liver, or cardiac fibrosis, more preferably, wherein the fibrosis is selected from muscle fibrosis. Preferably, the compound is an exon-skipping oligonucleotide.

Preferably, an effective amount of a compound that specifically reduces the amount or activity of TGFBR1 protein in a cell of the animal is also provided. An exon-skipping ACVR1B oligonucleotide may be given separately, sequentially or simultaneously with an exon-skipping TGFBR1 oligonucleotide. The oligonucleotides may be administered separately or together in the same composition. FIGS. 19C and 19D; FIGS. 20C and 20D; and FIG. 21 demonstrate the surprising effects of a combination of ACVR1B and TGFBR1 inhibition on fibrosis.

Pharmaceutical compositions are also provided for use in inhibiting, decreasing and/or preventing the development of fibrosis in the animal, wherein the composition comprises a pharmaceutically acceptable excipient and a compound that specifically reduces the amount or activity of TGFBR1 protein in a cell of an animal and/or a compound that specifically reduces the amount or activity of ACVR1B protein in a cell of an animal. Preferably, the compounds are exon-skipping oligonucleotides and, more preferably, the composition comprises both an ACVR1B exon-skipping oligonucleotide and a TGFBR1 exon-skipping oligonucleotide. Preferably, the pharmaceutical compositions include a dystrophin exon-skipping oligonucleotide, as described herein.

As used herein, the term “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue. As used herein, the term “fibrosis” is used synonymously with “fibroblast accumulation and collagen deposition.”

The methods and compounds included herein are useful for treating or preventing the development of fibrosis as well as preventing an exacerbation of fibrosis. Fibrocyte formation and/or fibroblast proliferation may be prevented or reduced. In addition to decreasing the proliferation of fibroblasts, the proliferation of myofibroblasts can also be decreased as well as the differentiation toward the myofibroblast phenotype. Myofibroblasts can differentiate from fibroblasts and can be distinguished from fibroblasts by the presence of stress fibers containing the alpha-smooth muscle actin isoform.

The effectiveness of the treatment of fibrosis may be determined, e.g., by monitoring molecular markers of fibrosis. Such markers include SERPINE1 (also known as Plasminogen activator inhibitor-1 (Pai1)) and other well-known markers of fibrosis such as alpha-SMA and those described in US 2009/0117591. Non-invasive fibrosis staging tests may also be used such as determining the aspartate aminotransferase/platelet ratio (APR1), Fibrotest (Biopredictive, Paris, France), i.e., a fibrosis index that combines the evaluation of five indirect serum fibrosis markers, and FibroScan (Echosens, Paris, France) (L. Sandrin et al. (2003), “Transient elastography: a new non-invasive method for assessment of hepatic fibrosis,” Ultrasound Med. Biol. 29:1705-1713). Methods for quantifying the level of fibrosis are also described in US 2010/0209940.

In one aspect, described is a method for decreasing the proliferation of fibroblast cells and/or differentiation of cells toward myofibroblast phenotype comprising providing the cells a compound that specifically reduces the amount or activity of TGFBR1 protein in the cell, preferably, wherein the fibroblast cell is a myofibroblast. Preferably, the method is an in vitro method.

In one aspect, described is a method for decreasing the proliferation of fibroblast cells and/or differentiation of cells toward myofibroblast phenotype comprising providing the cells a compound that specifically reduces the amount or activity of ACVR1B protein in the cell, preferably, wherein the fibroblast cell is a myofibroblast. Preferably, the method is an in vitro method.

As used herein, “decreasing proliferation” refers to a reduction in proliferation over control cells of at least 10%, 20%, 30%, 40%, 50%, 60%, 70%, 80%, 90%, or more. As used herein, “decreasing differentiation” refers to a reduction in differentiation toward a myofibroblast phenotype over control cells of at least 10%, 20%, 30%, 40%, 50%, 60%, 70%, 80%, 90%, or more. In some embodiments, the cells have been or are provided with a stimulus for fibrocyte proliferation or myofibroblast differentiation, such as, e.g., TGF-beta. The reduction in proliferation is as compared to cell proliferation under similar conditions only without treatment by the compound, e.g., as compared to treatment with a control or “scrambled” oligonucleotide.

A most preferred compound to be used in combination with the methods and compounds as disclosed herein is a dystrophin exon-skipping nucleic acid. Dystrophin plays an important structural role in the muscle fiber, connecting the extracellular matrix and the cytoskeleton. The N-teiininal region binds actin, whereas the C-terminal end is part of the dystrophin glycoprotein complex (DGC), which spans the sarcolemma. In the absence of dystrophin, mechanical stress leads to sarcolemmal ruptures, causing an uncontrolled influx of calcium into the muscle fiber interior, thereby triggering calcium-activated proteases and fiber necrosis.

Duchenne muscular dystrophy (DMD) and Becker muscular dystrophy (BMD) are both caused by mutations in the DMD gene, which codes for dystrophin. Frame-shifting mutations in the DMD gene result in dystrophin deficiency in muscle cells. This is accompanied by reduced levels of other DGC proteins and results in the severe phenotype found in DMD patients. Mutations in the DMD gene that keep the reading frame intact, generate shorter, but partly functional dystrophins associated with the less severe BMD.

Dystrophin exon-skipping nucleic acids have been developed as a treatment for muscular dystrophy, e.g., DMD and BMD. These nucleic acids are designed to skip an exon in order to restore a disrupted reading frame, resulting in the generation of internally deleted, but largely functional, dystrophin protein. The combination of RNAi against ACVR2B with exon skipping of dystrophin was recently reported (Dumonceaux et al. (2010), Molecular Therapy 18:881-887). The base-pairing sequence of a U7 snRNA was modified to bind to dystrophin pre-mRNA and to induce exon skipping of exon 23 of dystrophin (Brun et al. (2003), Cell. Mol. Life Sci. 60:557-566). The specific U7 snRNA construct was provided in adeno-associated vectors. ACVR2B is the receptor for a number of signaling pathways including those mediated by Activin A, Inhibin A/B, Nodal, BMP2, BMP6/7, GDF5, GDF11, and myostatin. Inhibition of ACVR2B function as described in Dumonceaux et al., therefore, affects a wide number of signaling pathways, which may lead to negative side-effects if used as a treatment. Additionally, myostatin signaling is not completely blocked by ACVR2B inhibition as signaling may also occur through ACVR2A. The combination of compounds that specifically reduce the amount or activity of ACVR1B protein and/or compounds that specifically reduces the amount or activity of TGFBR1 protein in a cell with a dystrophin exon-skipping nucleic acid may, therefore, provide not just an alternative to the treatment of muscle disorders, but an improvement over previous known methods.

Dystrophin exon-skipping nucleic acids disclosed herein include oligonucleotides comprising between 15 and 100 nucleotides, preferably between 15 and 40 nucleotides, complementary to a dystrophin pre-mRNA, wherein the oligonucleotide binds dystrophin pre-mRNA and modulates splicing of the mRNA. As used herein, the dystrophin exon-skipping nucleic acid is referred to as a “dystrophin exon-skipping oligonucleotide.” Dystrophin exon-skipping nucleic acids also include vectors, preferably viral vectors, comprising the dystrophin exon-skipping oligonucleotide, as well as nucleic acid molecules comprising dystrophin exon-skipping oligonucleotides operably linked to a promoter sequence. Such additional embodiments are discussed further below. Preferably, a dystrophin exon-skipping nucleic acid is a dystrophin exon-skipping oligonucleotide.

Dystrophin exon-skipping nucleic acids, suitable for use in the methods and compositions disclosed herein, as well as methods for designing and making the nucleic acids have been described in, e.g., Brun et al. (Cell. Mol. Life. Sci. (2003) 60:557-566), WO02/024906, WO2004/083446, WO2006/112705, WO2007/135105; and WO2009/139630, the contents of each of which are incorporated by reference in their entirety. The disclosure herein further provides various modifications that may be incorporated into an exon-skipping nucleic acid.

Preferably, a dystrophin exon-skipping oligonucleotide comprises or consists of a sequence that is complementary to at least part of dystrophin pre-mRNA exon 51, 44, 45, 53, 46, 43, 2, 8, 50 and/or 52, more preferably to exon 51 or 44, the part having at least 13 nucleotides. Preferably, a dystrophin exon-skipping oligonucleotide comprises or consists of a sequence selected from SEQ ID NO:3 to SEQ ID NO:284 of WO 2009/054725, hereby incorporated herein by reference; more preferably, the oligonucleotide comprises or consists of SEQ ID NO:204 of WO 2009/054725, i.e., UCAAGGAAGAUGGCAUUUCU. Preferably, a dystrophin exon-skipping oligonucleotide comprises or consists of a sequence selected from SEQ ID NOS:5, 45-49, 51, or 54 of WO 2009/139630, hereby incorporated by reference. More preferably, a dystrophin exon-skipping oligonucleotide comprises or consists of M23D (+02-18) (GGCCAAACCUCGGCUUACCU (SEQ ID NO:8)).

The compounds described herein for reducing the amount or activity of either ACVR1B or TGFBR1 may be used conjointly with a dystrophin exon-skipping oligonucleotide. Though not wishing to be bound by theory, the effect of reducing fibrotic tissue and increasing the amount of muscle fibers via the reduction of ACVR1B and/or TGFBR1 activity is believed to be beneficial in increasing the therapeutic efficacy of dystrophin exon-skipping oligonucleotides. Patients with highly dystrophic muscle show very low levels of dystrophin increase in response to treatment with dystrophin exon-skipping oligonucleotides in comparison with patients with less dystrophic muscle and less fibrotic tissue (van Deutekom et al. (2007), New England Journal of Medicine 357:2677). The combination therapy is thus especially useful for the treatment of a muscular dystrophy.

Dystrophin exon-skipping oligonucleotides and compounds for reducing the amount or activity of ACVR1B and/or compounds for reducing the amount or activity of TGFBR1 may be provided separately, sequentially, or simultaneously. In some embodiments, treatment with compound for reducing the amount or activity of either ACVR1B and/or TGFBR1 may be followed by a treatment with a dystrophin exon-skipping oligonucleotide. The treatments may be separated by days or weeks, but preferably, the treatments overlap.

In a further aspect, pharmaceutical compositions are provided comprising a dystrophin exon-skipping oligonucleotide and a compound that specifically reduces the amount or activity of ACVR1B protein in a cell and/or a compound that specifically reduces the amount or activity of TGFBR1 protein in a cell. Compositions encompassed by the present invention, therefore, include those in which the active ingredients are an ACVR1B exon-skipping oligonucleotide and a dystrophin exon-skipping oligonucleotide; a TGFBR1 exon-skipping oligonucleotide and a dystrophin exon-skipping oligonucleotide; and a ACVR1B exon-skipping oligonucleotide, a TGFBR1 exon-skipping oligonucleotide, and a dystrophin exon-skipping oligonucleotide.

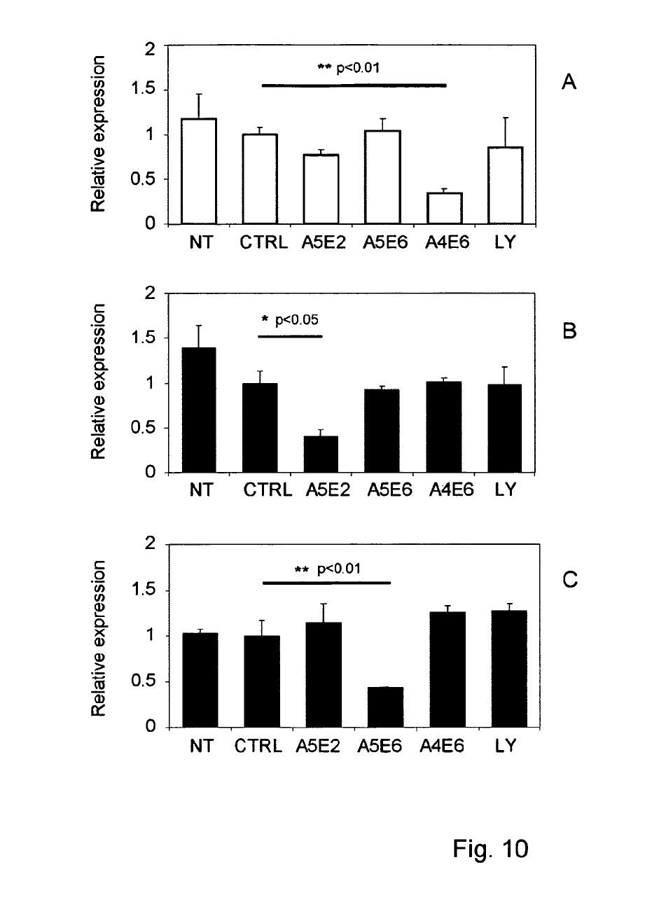

In one aspect of the disclosure, compounds are provided that specifically reduce the amount or activity of TGFBR1 protein in a cell. The compounds are specific for TGFBR1 such that the compounds do not significantly reduce the amount or activity of other proteins, in particular, ACVR1B (see, e.g., FIGS. 1 and 10). In a further aspect of the disclosure, compounds are provided that specifically reduce the amount or activity of ACVR1B protein in a cell. The compounds are specific for ACVR1B such that the compounds do not significantly reduce the amount or activity of other proteins, in particular TGFBR1 (see, e.g., FIGS. 1 and 10). These compounds are useful in a variety of methods, including those described herein.

A reduction in the amount or activity of the protein results in a reduction of ligand signaling, e.g., signaling via a member of the TGF-beta superfamily, such as myostatin or TGF-beta. A reduction in the amount of the protein may be a reduction of at least 5, at least 10, at least 20, at least 30, at least 40, at least 50, at least 60, at least 70, at least 80, or at least 90% of the protein in a cell. The reduction of protein may be observed by either a reduction in protein or a reduction in mRNA transcript (FIGS. 1 and 10). A reduction in the activity of the protein may be a reduction of at least 5, at least 10, at least 20, at least 30, at least 40, at least 50, at least 60, at least 70, at least 80, or at least 90% of the activity in a cell (FIGS. 2 and 11). The reduction of protein amount or activity is as compared to a cell under similar conditions only without treatment by the compound, e.g., as compared to treatment with a control or “scrambled” oligonucleotide.

Compounds for reducing the amount or activity of the proteins include molecules that specifically bind the proteins and inhibit their respective functions or lead to their degradation (e.g., antibodies, small chemical compounds) as well as compounds that affect mRNA transcription, mRNA splicing, or protein translation (e.g., antisense oligonucleotides and siRNA).

In some embodiments, compounds for reducing the amount or activity of ACVR1B or TGFBR1 protein are antibodies. The term “antibody” includes, for example, both naturally occurring and non-naturally occurring antibodies, polyclonal and monoclonal antibodies, chimeric antibodies and wholly synthetic antibodies and fragments thereof, such as, for example, the Fab′, F(ab′)2, Fv or Fab fragments, or other antigen-recognizing immunoglobulin fragments. Preferably, the antibody binds ACVR1B or TGFBR1 and prevents the interaction with ligand and/or a type II receptor. Methods of making antibodies are well known in the art.

Preferably, the compounds for reducing the amount or activity of ACVR1B or TGFBR1 protein are nucleic acid compounds. These nucleic acid compounds are preferably selected from an oligonucleotide as described herein; a vector comprising the oligonucleotide; and a nucleic acid comprising the oligonucleotide, preferably, the oligonucleotide, is operably linked to a promoter sequence. Preferably, the oligonucleotide is an oligonucleotide that modulates splicing of the target mRNA or an siRNA molecule.

The design and production of siRNA molecules is well known to one of skill in the art (P. B. Hajeri and S. K. Singh (2009), Drug. Discov. Today 14 (17-18):851-8). Methods of administration of therapeutic siRNA is also well-known to one of skill in the art (N. Manjunath and D. M. Dykxhoorn (2010), Discov. Med. May, 9 (48):418-30; J. Guo et al. (2010), Mol. Biosyst. July, 15, 6 (7):1143-61). Exemplary siRNA molecules are also provided in the examples herein.

In a particularly preferred embodiment, the compounds for use in the methods disclosed herein are oligonucleotides that modulate the splicing of ACVR1B or TGFBR1, respectively. These oligonucleotides are herein referred to as “exon-skipping oligonucleotides”). It may not be desired to completely reduce the activity of ACVR1B or TGFBR1. The use of exon-skipping oligonucleotides, either alone or as part of a larger nucleic acid, has the advantage that these molecules can be easily titrated to obtain a desired level of knock-down since these molecules are not catalytically active.

Oligonucleotides useful herein can be grouped into several different categories based on their mechanism of action. Conventional antisense oligonucleotides (AONs) mediate their effects, either by an RNaseH-dependent or independent mechanism. RNaseH is an enzyme that hydrolyzes the RNA strand of an RNA/DNA duplex, thus degrading the targeted mRNA.

AONs having an RNaseH-independent mechanism of action are thought to exert their effects by steric blocking. Such AONs may inhibit ribosomal translation and are thus usually targeted by the 5′ coding region. Such AONs may be referred to as RNaseH-resistant to refer to their inability to activate RNaseH when bound to an RNA molecule.

Another type of RNaseH-independent AONs are exon-skipping oligonucleotides. As already described, exon-skipping oligonucleotides bind to pre-mRNA and modulate splicing such that one or more exons are skipped in the resulting mRNA. Exon skipping may lead to an in frame deletion, resulting in a truncated protein or protein lacking internal amino acids or skipping may lead to a premature stop codon resulting in nonsense-mediated decay. Exon skipping does not depend on the action of a ribonuclease, such as a double-stranded ribonuclease or RNase H. Their mechanism of action is thus independent of a double-stranded ribonuclease or RNase H.

AONs are subject to degradation by endonucleases and exonucleases. Chemical modifications have been used to improve AON stability. The modifications for RNaseH-dependent AONs are limited, as many of the newly developed modifications are not compatible with RNaseH activity. Modifications such as 2′MeO, 2′MeOE, BNA/LNA, ENA, N3′-P5′ phosphoroamidate NP, MePO linkages, PNAs, and morpholinos fail to activate RNaseH. Alternatively, other modifications such as GAPmers may be used in RNaseH-dependent AONs.

RNA interference oligonucleotides may also be used to reduce the amount or activity of a target protein. RNA interference refers to a single-stranded RNA-type molecule binding to a target RNA molecule and recruiting a ribonuclease that degrades the target RNA. RNA interference molecules include double-stranded siRNAs, as well as miRNAs and shRNAs, both of which form a double-stranded hairpin structure. This double-stranded structure (provided either in the same or by two separate molecules) is believed to increase the efficiency of the RISC complex as well as prevent degradation by single-strand-specific ribonucleases (Martinez et al. (2002), Cell 110:563-574). Modifications have also been developed to improve stability of RNAi molecules. These modifications must be compatible with the formation of the RISC complex as well as the function of the single-strand-specific ribonucleases.

Further details regarding the differences between oligonucleotides and their modifications may be found in Pan and Clawson (J. Cell. Bio. 2006 98:14-35) and Dias and Stein (Mol. Cancer. Ther. 2002 1:347-355), which are hereby incorporated by reference in their entirety.

Exon-skipping AONs have a number of advantages over RNAi. First, a major obstacle in applying RNA interference strategies is the ability to deliver therapeutically active compounds to the target site effectively and safely (Kinouchi et al. (2008), Gene Therapy 15:1126-1130). Delivery agents are currently being developed for delivering RNAi molecules, although none have yet been approved for use in humans. In contrast, exon-skipping oligonucleotides do not require special delivery agents in order to achieve therapeutic effects (see, e.g., FIGS. 15 and 19) and their effects may be further enhanced by covalent binding to muscle-targeting peptides such as those described in C. A. Betts et al. ((2012), Methods Mol. Biol. 867:415-35). Second, exon-skipping oligonucleotides are single-stranded and do not induce an immunogenic response in contrast to double-stranded RNAi-like oligonucleotides.

Exon-skipping AONs have the advantage over RNaseH-dependent AONs that additional modifications (such as 2′MeO, 2′MeOE, and morpholinos) can be used to increase stability or efficacy without negatively affecting their mechanism of action. In preferred embodiments, the exon-skipping AONs are RNaseH incompatible, i.e., they do not activate RNaseH when bound to their target pre-mRNA.

Antisense oligonucleotides for exon skipping typically enable skipping of an exon or the 5′ or 3′ part of it. Antisense oligonucleotides can be directed toward the 5′ splice site, the 3′ splice, to both splice sites, to one or more exon-internal sites and to intron sequences, for instance, specific for the branch point. The latter enables skipping of the upstream exon. An oligonucleotide is said to be directed toward an exon internal sequence if the complementarity region that contains the sequence identity to the reverse complement of the target pre-mRNA is within the exon boundary. Methods for designing exon-skipping oligonucleotides have been described (Aartsma-Rus et al. (2009), Mol. Ther. 17(3):548).

Preferably, the oligonucleotides modulate RNA splicing such that the skipping of one or more exons does not alter the reading frame of the mRNA transcript, more preferably, wherein exon skipping results in the production of a dominant negative protein. As used herein, a dominant negative protein affects the normal, wild-type protein, for example, by interacting with the same elements as the wild-type product, but lacking the full function of the wild-type protein. A dominant negative of ACVR1B or TGFBR1 includes a protein that lacks the ligand binding domain while retaining the kinase domain. The resulting protein may reduce signaling capacity by dimerizing with functional type II receptors. A dominant negative of ACVR1B or TGFBR1 also includes a protein that lacks the kinase domain but retains the ligand binding domain and can function as a scavenging receptor for its natural ligands.

Preferably, provision of the oligonucleotide results in the skipping of exon 2, 4, 6, or 7, more preferably, exon 2 or exon 6 of ACVR1B or TGFBR1 (FIG. 9). The skipping of exon 2 results in a protein lacking the ligand binding domain while retaining the kinase domain. The resulting protein may, therefore, function as a scavenging receptor for its natural ligands. The skipping of exon 4 results in a transcript lacking the Gly-Ser domain making the receptor dysfunctional. The skipping of either of exons 6 or 7 results in the disruption of the open reading frame, introducing a premature stop codon and resulting in a truncated kinase domain, making the receptor dysfunctional.

Preferably, an ACVR1B exon-skipping oligonucleotide comprises or consists of A4E6 (UGGUGUCAGUGACCGCAUCAU (SEQ ID NO:1)) or ViMo A4E6 (TCTATGGTGTCAGTGACCGCATCAT (SEQ ID NO:2)) (see Table 1). Preferably, an ACVR1B exon-skipping oligonucleotide comprises or consists of A4E6a (UGGUGUCAGUGACUGCAUCAU (SEQ ID NO:9) or hA4E6b (GUUUGGUCCCCACCCUCUGAUU (SEQ ID NO:10)) (see Table 2). Preferably, a TGFBR1 exon-skipping oligonucleotide comprises or consists of A5E2 (GCAGUGGUCCUGAUUGCAGCA (SEQ ID NO:3)), A5E6 (AUCUGUGGCAGAAUCAUGUCU (SEQ ID NO:4)), or ViMo A5E2 (GCAGTGGTCCTGATTGCAGCAATAT (SEQ ID NO:5)) (see Table 1). Preferably, a TGFBR1 exon-skipping oligonucleotide comprises or consists of A5E2a (UGUACAGAGGUGGCAGAAACA (SEQ ID NO:11)) or hA5E2b (GCAAUGGUCCUGAUUGCAGCA (SEQ ID NO:12)) (see Table 2).

The term pre-mRNA refers to a non-processed or partly processed precursor mRNA that is synthesized from a DNA template in the cell nucleus by transcription. As used herein, an oligonucleotide that modulates the splicing of an mRNA means that at least 1%, 10%, 20%, 30%, 40%, 50%, 60%, 70%, 80%, 90% or more of the mRNA encoding the targeted protein in a cell will not contain a skipped exon sequence (modified/(modified+unmodified) mRNA). This is preferably assessed by quantitative PCR as described in the examples.

Exon-skipping oligonucleotides have between 15 and 100, preferably 15 and 40, nucleotides complementary to their target pre-mRNA (i.e., ACVR1B or TGFBR1). Preferably, the length of the complementary part of the exon-skipping oligonucleotide is at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. Preferably, additional flanking sequences are used to modify the binding of a protein to the molecule or oligonucleotide, or to modify a thermodynamic property of the oligonucleotide, more preferably, to modify target RNA binding affinity. An exon-skipping oligonucleotide as disclosed herein may further comprise additional nucleotides that are not complementary to the target site on the target pre-mRNA. Depending on the chemistry of the backbone as indicated hereinabove, an exon-skipping oligonucleotide contains between 15 and 25 nucleotides. An exon-skipping oligonucleotide with a morpholino backbone typically contains a stretch of between 25 and 40 nucleotides. When reference is made herein to a range of nucleotides, this range includes the number(s) mentioned. Thus, by way of example, when reference is made to a stretch of between 15 and 40, this includes 15 and 40.

Preferably the complementary part is at least 50% of the length of the oligonucleotide of the invention, more preferably at least 60%, even more preferably at least 70%, even more preferably at least 80%, even more preferably at least 90% or even more preferably at least 95%, or even more preferably 98% and most preferably up to 100% of the length of the oligonucleotide, with the putative exception of deliberately introduced specific mismatches, e.g., for down-regulating affinity when necessary.

The term complementarity is used herein to refer to a stretch of nucleic acids that can hybridize to another stretch of nucleic acids under physiological conditions. It is thus not absolutely required that all the bases in the region of complementarity are capable of pairing with bases in the opposing strand. For instance, when designing the oligonucleotide one may want to incorporate for instance a residue that does not base pair with the base on the complementary strand. Mismatches may to some extent be allowed, if under the circumstances in the cell, the stretch of nucleotides is capable of hybridizing to the complementary part. In a preferred embodiment a complementary part (either to the open or to the closed structure) comprises at least 3, and more preferably at least 4 consecutive nucleotides. The complementary regions are preferably designed such that, when combined, they are specific for the exon in the pre-mRNA. Such specificity may be created with various lengths of complementary regions as this depends on the actual sequences in other (pre-)mRNA in the system. The risk that also one or more other pre-mRNA will be able to hybridize to the oligonucleotide decreases with increasing size of the oligonucleotide. It is clear that oligonucleotides comprising mismatches in the region of complementarity but that retain the capacity to hybridize to the targeted region(s) in the pre-mRNA, can be used in the present invention. However, preferably at least the complementary parts do not comprise such mismatches as these typically have a higher efficiency and a higher specificity, than oligonucleotides having such mismatches in one or more complementary regions. It is thought that higher hybridization strengths (i.e., increasing number of interactions with the opposing strand) are favorable in increasing the efficiency of the process of interfering with the splicing machinery of the system. Preferably, the complementarity is between 90 and 100%. In general this allows for approximately 1 or 2 mismatch(es) in an oligonucleotide of around 20 nucleotides.

In preferred embodiments, an inosine has been introduced in the oligonucleotide sequence to replace a guanine, adenine, or a uracil. Inosine may be used to form a wobble base pair. A wobble base pair is a G-U and I-U/I-A/I-C pair fundamental in RNA secondary structure. Its thermodynamic stability is comparable to that of the Watson-Crick base pair. Advantages to using an inosine base include the ability to design an AON that spans a single nucleotide polymorphism (SNP), without worry that the polymorphism will disrupt the ability of the oligonucleotide to bind to its target. Therefore in the invention, the use of such a base allows to design an oligonucleotide that may be used for an individual having a SNP within the pre-mRNA stretch which is targeted by an oligonucleotide of the invention. Further advantages are described in WO2011/078672, which is hereby incorporated by reference.

With respect to exon-skipping oligonucleotides that also contain additional nucleotides, the total number of nucleotides typically does not exceed 50, and the additional nucleotides preferably range in number from between 5 and 25, preferably from between 20 and 25, more preferably from between 15 and 25. The additional nucleotides typically are not complementary to the target site on the pre-mRNA but may be complementary to another site on the pre-mRNA or may serve a different purpose and not be complementary to the target pre-mRNA, i.e., less than 95% sequence identity of the additional nucleotides to the reverse complement of the target pre-mRNA.

In a preferred embodiment, the nucleic acid compound, preferably the exon-skipping oligonucleotide, comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene-containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six-membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report demonstrated triplex formation by a morpholino oligonucleotide and, because of the non-ionic backbone, these studies showed that the morpholino oligonucleotide was capable of triplex formation in the absence of magnesium.

A modified backbone is typically preferred to increase nuclease resistance of the exon-skipping oligonucleotide, the target RNA or the exon-skipping oligonucleotide/target RNA hybrid or a combination thereof A modified backbone can also be preferred because of its altered affinity for the target sequence compared to an unmodified backbone. An unmodified backbone can be RNA or DNA, preferably it is an RNA backbone. Preferably, the oligonucleotides provided herein are morpholino oligonucleotides comprising a covalently linked delivery moiety, which is comprised of an octa-guanidine dendrimer (e.g., ViVo-morpholinos from Gene Tools, Inc.).

It is further preferred that the linkage between the residues in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991), Science 254, 1497-1500). PNA-based molecules are true mimics of DNA molecules in temis of base-pair recognition. The backbone of the PNA is composed of 7V-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005), Chem. Commun. 495-497). Since the backbone of a PNA molecule contains no charged phosphate groups, PNA-RNA hybrids are usually more stable than RNA-RNA or RNA-DNA hybrids, respectively (Egholm et al. (1993), Nature 365, 566-568).

A further preferred backbone comprises a morpholino nucleotide analog or equivalent, in which the ribose or deoxyribose sugar is replaced by a six-membered morpholino ring. A most preferred nucleotide analog or equivalent comprises a phosphorodiamidate morpholino oligomer (PMO), in which the ribose or deoxyribose sugar is replaced by a six-membered morpholino ring, and the anionic phosphodiester linkage between adjacent morpholino rings is replaced by a non-ionic phosphorodiamidate linkage.

In yet a further embodiment, a nucleic acid molecule as disclosed herein comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred exon-skipping oligonucleotide comprises one or more sugar moieties that are mono- or di-substituted at the 2′, 3′ and/or 5′ position such as a —OH; —F; substituted or unsubstituted, linear or branched lower (Cl—ClO) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; -aminooxy; methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or a deoxyribose or a derivative thereof. Such preferred derivatized sugar moieties comprise Locked Nucleic Acid (LNA), in which the 2′-carbon atom is linked to the 3′ or 4′ carbon atom of the sugar ring, thereby forming a bicyclic sugar moiety. A preferred LNA comprises 2′-0,4′-C-ethylene-bridged nucleic acid (Morita et al. (2001), Nucleic Acid Res. Supplement No. 1: 241-242). These substitutions render the nucleotide analogue or equivalent RNase H and nuclease-resistant and increase the affinity for the target RNA. Other means for increasing the resistance to RNase H, or rather render the oligonucleotides RNase H in compatible, are described in U.S. Pat. No. 5,149,797, which is hereby incorporated herein by reference in its entirety.

It is understood by a skilled person that it is not necessary for all positions in an oligonucleotide to be modified uniformly. In addition, more than one of the aforementioned analogues or equivalents may be incorporated in a single oligonucleotide or even at a single position within an oligonucleotide. In certain embodiments, an oligonucleotide of the invention has at least two different types of analogues or equivalents.

A preferred exon-skipping oligonucleotide comprises a T-O alkyl phosphorothioate antisense oligonucleotide, such as 2′-O-methyl modified ribose (RNA), 2′-0-ethyl modified ribose, 2′-O-propyl modified ribose, and/or substituted derivatives of these modifications such as halogenated derivatives. A most preferred AON according to the invention is comprised of 2′-0-methyl phosphorothioate ribose.

A nucleic acid compound as described herein can be linked to a moiety that enhances uptake of the oligonucleotide in cells. Examples of such moieties are cholesterols, carbohydrates, vitamins, biotin, lipids, phospholipids, cell-penetrating peptides including, but not limited to, antennapedia, TAT, transportan and positively charged amino acids such as oligoarginine, poly-arginine, oligolysine or polylysine, antigen-binding domains such as provided by an antibody, a Fab fragment of an antibody, or a single-chain antigen-binding domain such as a cameloid single domain antigen-binding domain.

Preferably, additional flanking sequences are used to modify the binding of a protein to the exon-skipping oligonucleotide, or to modify a thermodynamic property of the exon-skipping oligonucleotide, more preferably, to modify target RNA binding affinity.

The nucleic acid compound, e.g., siRNA or an exon-skipping oligonucleotide, may be provided to a cell as part of a gene delivery vehicle. Such a vehicle is preferably a liposome or a viral gene delivery vehicle. Liposomes are well known in the art and many variants are available for gene transfer purposes. Various viral gene delivery are currently used to transfer genes into target cells. In the present disclosure, it is preferred to use those viral vectors that do not express their own genes but only the transferred genes. The nucleic acid compound may be present as such in the gene delivery vehicle. In a viral vector, the nucleic acid compound is preferably provided as an expression cassette wherein the expression cassette encodes a transcript comprising the compound.

Accordingly, vectors are provided comprising one or more oligonucleotides as described herein. A “vector” is a recombinant nucleic acid construct, such as plasmid, phase genome, virus genome, cosmid, or artificial chromosome, to which another DNA segment may be attached. The tee n “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus, pox, baculovirus, vaccinia, herpes simplex, Epstein-Barr and adenovirus vectors, as set forth in greater detail below. Vector sequences may also contain one or more regulatory regions, and/or selectable markers useful in selecting, measuring, and monitoring nucleic acid transfer results (transfer to which tissues, duration of expression, etc.).

Preferably, the vector sequences provide delivery of the exon-skipping oligonucleotide to the appropriate cells. A preferred delivery vehicle is a viral vector such as an adeno-associated virus vector (AAV), or a retroviral vector such as a lentivirus vector and the like (A. Goyenvalle, et al. (2004), Science 306(5702):1796-9).

Also plasmids, artificial chromosomes, plasmids suitable for targeted homologous recombination and integration in the human genome of cells may be suitably applied for delivery of a nucleic acid compound as defined herein. Preferred are those vectors wherein transcription is driven from PolIII promoters, and/or wherein transcripts are in the form fusions with U1 or U7 transcripts, which yield good results for delivering small transcripts. It is within the skill of the artisan to design suitable transcripts. Preferred are PolIII driven transcripts.

The disclosure further provides a compound that specifically reduces the amount or activity of ACVR1B protein or specifically reduces the amount or activity of TGFBR1 protein in a cell of an animal, wherein the compound is a nucleic acid compound comprising the respective exon-skipping oligonucleotide as disclosed herein. The nucleic acid molecules do not encompass the full-length ACVR1B or TGFBR1 sequences as these do not specifically reduce the amount or activity of the respective proteins. The exon-skipping oligonucleotide of the nucleic acid compound is, preferably, operably linked to a promoter sequence, preferably a Pol II or Pol III promoter. Preferably, the nucleic acid molecule further comprises U7 snRNA sequences. The use of U7 snRNA as an exon-skipping therapy has been discussed herein (Brun et al. (2003), Cell. Mol. Life. Sci. 60:557-566). Additional sequences of the nucleic acid compounds include vector, e.g., viral vector, sequences.

The disclosure further contemplates the combination of the methods and compounds described herein with additional treatments. The methods and compounds described herein may be combined with any of the various treatments for fibrosis-related disorders and/or anti-inflammatory agents known to those skilled in the art. Anti-inflammatory agents include steroids, e.g., corticosteroids, penicillamine, colchicine, IL-12, IL-10, and IFN-gamma. See e.g., M. H. Beers and R. Berkow, eds., The Merck Manual 7th ed. Merck Research Laboratories, 1999. Anti-inflammatory agents include a steroid, a TNF-alpha inhibitor, a source of mIGF-1 and/or an antioxidant. However, any other compound able to reduce inflammation is also encompassed within the present disclosure.

Preferred combination treatments include the use of a steroid. Preferably, the steroid is a corticosteroid (glucocorticosteroid). Preferably, prednisone steroids (such as prednisone, prednizolone or deflazacort) are used in methods or compositions described herein. Exemplary dose ranges of (glucocortico) steroids are about 0.5-1.0 mg/kg/day, preferably about 0.75 mg/kg/day for prednisone and prednisolone, and about 0.4-1.4 mg/kg/day, preferably about 0.9 mg/kg/day for deflazacort.

Pharmaceutical preparations and compositions are also provided comprising compounds as described herein and, optionally, a dystrophin exon-skipping nucleic acid, and a pharmaceutically acceptable carrier, filler, preservative, adjuvant, solubilizer, diluent and/or excipient is also provided. Such pharmaceutically acceptable carrier, filler, preservative, adjuvant, solubilizer, diluent and/or excipient may, for instance, be found in Remington: The Science and Practice of Pharmacy, 20th Edition, Baltimore, Md.: Lippincott Williams & Wilkins, 2000.

In preferred embodiments, the compounds as described herein for specifically reducing the amount or activity of ACVR1B protein and/or of TGFBR1 protein are nucleic acid compounds. The compounds can be delivered “as is” to the cells. When administering the compounds thereof to an individual, it is preferred that the compound is dissolved in a solution that is compatible with the delivery method. For intravenous, subcutaneous, intramuscular, intrathecal and/or intraventricular administration, it is preferred that the solution is a physiological salt solution. Particularly preferred for a method of the disclosure is the use of an excipient that will aid in delivery of a compound as defined herein, preferably a nucleic acid compound, into a cell, preferably a muscle cell. Preferred are excipients capable of forming complexes, vesicles and/or liposomes that deliver such a compound as defined herein in a vesicle or liposome through a cell membrane. Many of these excipients are known in the art. Suitable excipients comprise polyethylenimine (PEI) or similar cationic polymers, including polypropyleneimine or polyethylenimine copolymers (PECs) and derivatives, ExGen 500, synthetic amphiphils (SAINT-18), LIPOFECTIN®, DOTAP and/or viral capsid proteins that are capable of self-assembly into particles that can deliver such compounds, preferably an oligonucleotide, to a cell, preferably a muscle cell. Such excipients have been shown to efficiently deliver nucleic acids to a wide variety of cultured cells, including muscle cells. Their high transfection potential is combined with an accepted low to moderate toxicity in terms of overall cell survival. The ease of structural modification can be used to allow further modifications and the analysis of their further (in vivo) nucleic acid transfer characteristics and toxicity.

LIPOFECTIN® represents an example of a liposomal transfection agent. It consists of two lipid components, a cationic lipid N-[1-(2,3 dioleoyloxy)propyl]-N,N,N-trimethylammonium chloride (DOTMA) (cp. DOTAP, which is the methylsulfate salt) and a neutral lipid dioleoylphosphatidylethanolamine (DOPE). The neutral component mediates the intracellular release. Another group of delivery systems are polymeric nanoparticles.

Polycations like diethylaminoethylaminoethyl (DEAE)-dextran, which are well known as DNA transfection reagent, can be combined with butylcyanoacrylate (PBCA) and hexylcyanoacrylate (PHCA) to formulate cationic nanoparticles that can deliver a compound as defined herein, preferably an oligonucleotide, across cell membranes into cells.

In addition to these common nanoparticle materials, the cationic peptide protamine offers an alternative approach to formulate a compound as defined herein, preferably an oligonucleotide, as colloids. This colloidal nanoparticle system can form so-called “proticles,” which can be prepared by a simple self-assembly process to package and mediate intracellular release of a compound as defined herein, preferably an oligonucleotide. The skilled person may select and adapt any of the above or other commercially available alternative excipients and delivery systems to package and deliver a compound as defined herein, preferably an oligonucleotide, for use in the current disclosure to deliver the compound for the treatment of muscle loss, insufficient muscle growth, or fibrosis.

Actual dosage levels of the pharmaceutical preparations described herein may be varied so as to obtain an amount of the active ingredient that is effective to achieve the desired therapeutic response for a particular patient, composition, and mode of administration, without being toxic to the patient. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds described herein at levels lower than that required in order to achieve the desired therapeutic effect and gradually increase the dosage until the desired effect is achieved.

In general, a suitable daily dose of a compound as described herein for specifically reducing the amount or activity of ACVR1B protein and/or of TGFBR1 protein will be that amount of the compound that is the lowest dose effective to produce a therapeutic effect. Preferably, the compounds are oligonucleotides and the concentration used ranges between about 0.3 to about 400 nM, even more preferably between about 1 to about 200 nM. If several oligonucleotides are used, this concentration may refer to the total concentration of oligonucleotides or the concentration of each oligonucleotide added. Preferably, the oligonucleotide is administered weekly at a dose of between 1 mg/kg to 10 mg/kg, preferably around 6 mg/kg intravenously.

Administration to an animal refers to administration of a therapeutically effective amount of a compound to an animal in need thereof. The animal may suffer from muscle loss or insufficient muscle growth, or may be at risk of suffering from muscle loss or insufficient muscle growth.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) other than the ones specifically identified, the additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10, more or less 1% of the value.

As used herein, the terms “treatment,” “treating,” and the like, refer to obtaining a desired pharmacologic and/or physiologic effect. The effect may be prophylactic in terms of completely or partially preventing a disorder or symptom thereof and/or may be therapeutic in terms of a partial or complete cure for a disorder and/or adverse symptom attributable to the disorder. “Treatment,” as used herein, covers any treatment of a disease in a mammal, particularly in a human, and includes: (a) increasing survival time; (b) decreasing the risk of death due to the disease; (c) preventing the disease from occurring in a subject that may be predisposed to the disease but has not yet been diagnosed as having it; (d) inhibiting the disease, i.e., arresting its development (e.g., reducing the rate of disease progression); (e) relieving the disease, i.e., causing regression of the disease; and (f) alleviating one or more symptoms of the disease.

As used herein, a therapeutic that “prevents” a disorder or condition is a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “animal” refers to animals, preferably mammals, more preferably humans. The term “mammal” includes primates (including humans), domesticated animals including dogs, cats, sheep, cattle, goats, pigs, mice, rats, and rabbits.

The term “therapeutically effective amount” means an amount of therapeutic agents, or a rate of delivery of such therapeutic agents, effective to facilitate a desired therapeutic effect. The precise desired therapeutic effect will vary according to the condition to be treated, the formulation to be administered, and a variety of other factors that are appreciated by those of ordinary skill in the art.

As used herein, silencing of signaling, e.g., myostatin, activin, or TGF-beta signaling, refers to reducing signaling by at least 30%, 40%, 50%, 60%, 70%, 80%, 90%, or more. Such a reduction in signaling would not occur if ACVR1B and TGFBR1 act redundantly to transmit the respective signal in a cell. The reduction, i.e., silencing of signaling, is the decrease in signaling as compared to a cell under similar conditions only without treatment by the compound, e.g., as compared to treatment with a control or “scrambled” oligonucleotide.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1

### Selective Knockdown of ACVR1B and TGFBR1

In order to elucidate the biological effects of ACVR1B and TGFBR1, it was necessary to specifically knock down the expression of these receptors. Commercial small molecule TGFBR1 inhibitors are effective antagonists of TGFBR1 activity, but also inhibit ACVR1B and ACVR1C activities. Therefore, specific siRNA-mediated knockdown of the individual receptors was used to distinguish between the effect of ACVR1B and TGFBR1 knockdown. FIG. 1 shows a ˜60-80% knockdown of ACVR1B transcript in C3H10 T1/2 and C2C12 cells, respectively, using 100 nM of siRNA targeting ACVR1B (siAcvr1b). Similarly, ˜90% knockdown of TGFBR1 transcript was achieved in both cell types using the same concentration of siRNA targeting TGFBR1 (siTgfbr1). Importantly, the siRNA targeting ACVR1B showed no effect on TGFBR1 expression and vice versa.

### Example 2

### Myostatin Signaling is Dependent on ACVR1B in the Myogenic Cells and TGFBR1 in the Non-Myogenic Cells

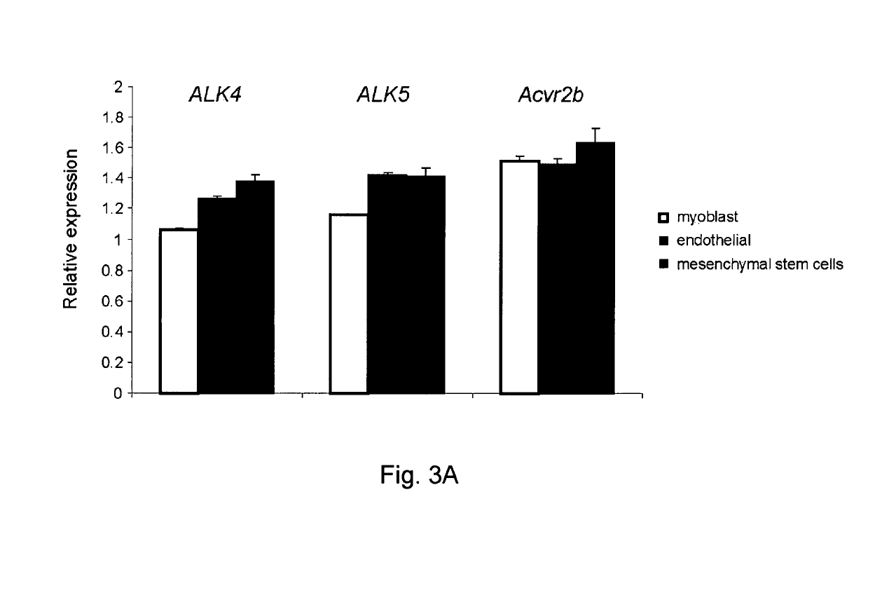

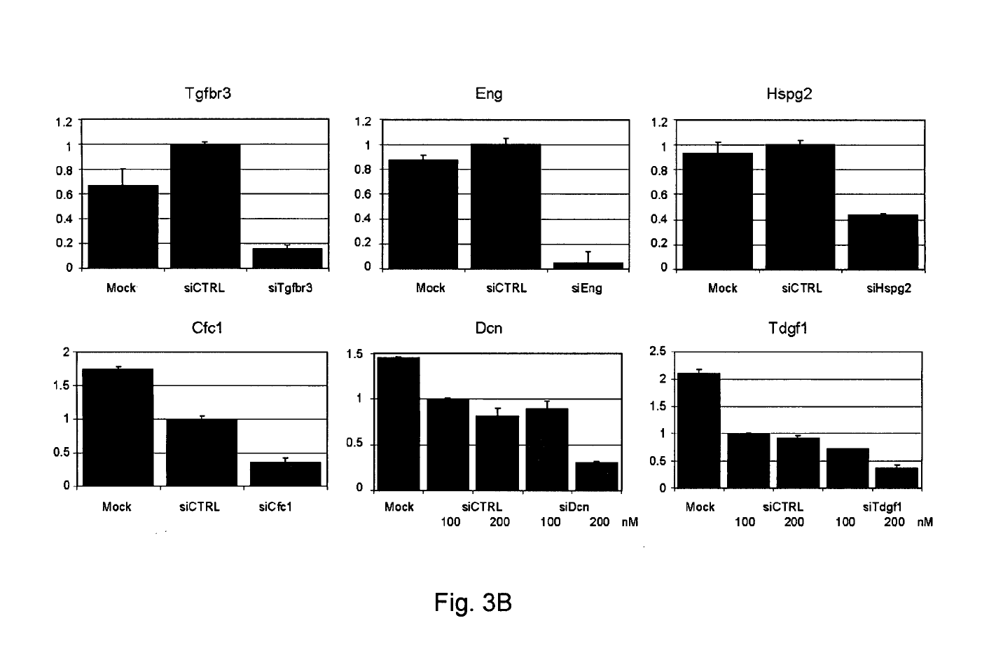

Next, the effect of ACVR1B or TGFBR1 knockdown on myostatin signaling in myogenic and non-myogenic cells was tested. Myogenic C2C12 cells were transiently transfected with CAGA-luciferase reporter construct, together with either the siRNA targeting ACVR1B or TGFBR1. Non-targeting siRNA and mock condition were included as controls. As shown in FIG. 2, Panel A, myostatin-induced CAGA luciferase was reduced to approximately five folds upon ACVR1B knockdown, whereas TGFBR1 knockdown had no effect compared to mock and control siRNA conditions. Surprisingly, an opposite effect was observed in the non-myogenic cells C3H10 T1/2 (FIG. 2, Panel B) and 3T3-L1 (not shown), in which only TGFBR1 knockdown decreased myostatin-induced CAGA luciferase to approximately two and one-half folds, whereas ACVR1B knockdown had no significant effect. Similar experiments were also performed using TGF-beta. In contrast to the results observed for myostatin signaling, only TGFBR1 knockdown reduced TGF-beta-mediated luciferase activity for approximately three folds in both cell types (FIGS. 2A and 2B). Importantly, the expression levels of ACVR1B and TGFBR1 were found to be comparable in the different cell types tested (FIG. 3A). Therefore, the differential effects of ACVR1B and TGFBR1 knockdown on myostatin signaling observed in these cell types is not due to differential expression of the receptors.

The findings were further validated by assessing the Smad2 phosphorylation in these cells. Upon ACVR1B knockdown, myostatin-induced Smad2 phosphorylation was reduced exclusively in the C2C12, whereas in C3H10 T1/2 cells, the levels of phosphorylated Smad2 were equal to the controls. In contrast, TGFBR1 knockdown had no effect on Smad2 phosphorylation in C2C12, but decreased in the C3H10 T1/2 cells. These results suggest the predominant use of ACVR1B as type I receptor by myostatin in the myogenic cells and the use of TGFBR1 in the non-myogenic cells.

### Example 3

### The Effect of Co-Receptor Knockdown in Myostatin and TGF-Beta Signaling in Myogenic Vs Non-Myogenic Cells

These differences in myostatin signaling between different cells prompted the hypothesis that a presence of type III receptors for myostatin underline the ACVR1B-mediated signaling in myogenic cells and TGFBR1-mediated signaling in non-myogenic cells. Various type III receptors have been proposed to modulate TGF-beta or Activin, such as, e.g., Endoglin (Eng), Betaglycan (Tgfbr3), TDGF1 (Tdgf1) and its family Cryptic (Cfc1), as well as modulatory proteins that have been associated with myostatin binding such as Decorin (Dcn) and Perlecan (Hspg2). It was hypothesized that one or more of these proteins might be involved in mediating myostatin signaling. siRNA was transiently transfected into C2C12 cells to separately silence these receptors and quantitatively assess transcript levels 48 hours post-transfection (FIG. 3B). Decreased expression (more than 50%) of betaglycan, endoglin, perlecan and cryptic mRNA levels was achieved using 100 nM siRNA targeting each gene. However, more than 50% knockdown of decorin and TDGF1 could only be achieved using 200 nM siRNA, most likely due to the efficiency or potency of the siRNA. Each siRNA targeting a specific co-receptor was subsequently co-transfected together with the CAGA-luciferase reporter constructs. Upon stimulation with myostatin for eight hours, TDGF1 knockdown resulted in a decrease in myostatin-induced CAGA luciferase signaling (FIG. 4, Panel A). Interestingly, this effect was observed exclusively in C2C12 myoblast and not in the C3H10T1/2 cells (FIG. 4, Panel B) or 3T3-L1 mouse pre-adipocytes cells. Additionally, betaglycan siRNA decreased myostatin-induced CAGA-luciferase signals in non-myogenic cells. As TDGF1 siRNA had no effects on betaglycan expression, the results indicate a differential mechanism of myostatin signaling in myogenic versus non-myogenic cells. To further validate the findings, TGF-beta-induced CAGA luciferase was assessed in parallel. TGFBR1 knockdown abrogated TGF-beta signaling in all cell types used (FIGS. 4C, 4D). However, no effect upon TDGF1 knockdown was observed. In contrast, TGF-beta signaling decreased upon betaglycan knockdown in both cell types (FIGS. 4C, 4D). Together, these results suggest that myostatin and TGF-beta signaling is differentially regulated in myogenic cells and that this difference is conferred by co-receptors, such as TDGF1.

### Example 4

### Specific Expression of TDGF1 in C2C12 Myoblast and Satellite Cells

The endogenous expression of TDGF1 in different panels of cells was compared. Surprisingly, the expression of TDGF1 is significantly higher in myoblasts as compared to mesenchymal stem cells C3H10 T1/2, preadipocytes 3T3-L1 and endothelial cells MEEC (FIGS. 5A and 5B).

### Example 5

### Opposing Modulatory Effect of TDGF1 for Myostatin and Activin Signaling in C2C12

To further understand the role of TDGF1 in myogenic cells, the effect of TDGF1 knockdown on activin and myostatin signaling was analyzed. TDGF1 siRNA was coupled with A3-luciferase reporter, a Smad2 activation-dependent reporter, to assess activin signaling in C2C12 myoblasts. Enhanced activin signaling was observed upon TDGF1 knockdown (FIG. 6A), which is coherent to the current knowledge for TDGF1 being an antagonist of Activin signaling (J. A. Kelber, et al. (2008), J. Biol. Chem. 22, 283(8):4490-500). Additionally, use was made of wild-type, ΔEGF and ΔCFC mutant TDGF1 (P. D. Gray et al. (2006), Mol. Cell. Biol. 26(24):9268-78) to investigate the role of TDGF1 in myostatin signaling in more detail. The N-teiininal EGF-like and C-terminal CFC domains of TDGF1 have been implicated in the ligand binding and association with the type I, respectively. 293T cells were transfected with the A3- and Renilla-luciferases, together with Fast-1 transcription factor. These cells have no endogenous expression of TDGF1. In addition, ACVR2B, ACVR1B and different TDGF1 constructs described above were expressed. In the activin-induced A3-Luciferase (FIG. 6B), decrease of Activin signaling was observed in the presence of wild-type TDGF1 and the delta EGF, but not when the delta CFC was co-transfected. However, as opposed to activin signaling, myostatin signaling was enhanced upon wild-type TDGF1 overexpression, thus confirming our observation with the TDGF1 knockdown in C2C12 that TDGF1 mediates myostatin signaling (FIG. 4, Panel A). In addition, decrease in the luciferase activity was observed upon transfection with mutant TDGF1 lacking the EGF or the CFC domains, suggesting the importance of these domains in modulating myostatin signaling (FIG. 6C).

### Example 6

### Down-Regulation of TDGF1 Enhances Myoblast Differentiation

To further assess the effect of TDGF1 in modulating myostatin signaling, the effect of TDGF1 knockdown on myoblast differentiation was investigated. ACVR1B, TGFBR1 or TDGF1 siRNA were transfected into C2C12 myoblasts. The effect of each siRNA in myogenic differentiation was assessed at different time points by means of immunofluorescent staining (FIG. 7A) and quantitative PCR (FIG. 7B). Enhanced myoblast differentiation was observed by means of myosin-positive myotubes in the panels of cells transfected with ACVR1B, TGFBR1 or TDGF1 siRNA, compared to the non-targeting control. These observations were supported by increased myogenin transcript expression (FIG. 7B).

### Example 7

### Proposed Mechanism of Co-Receptor Modulated Myostatin Signaling

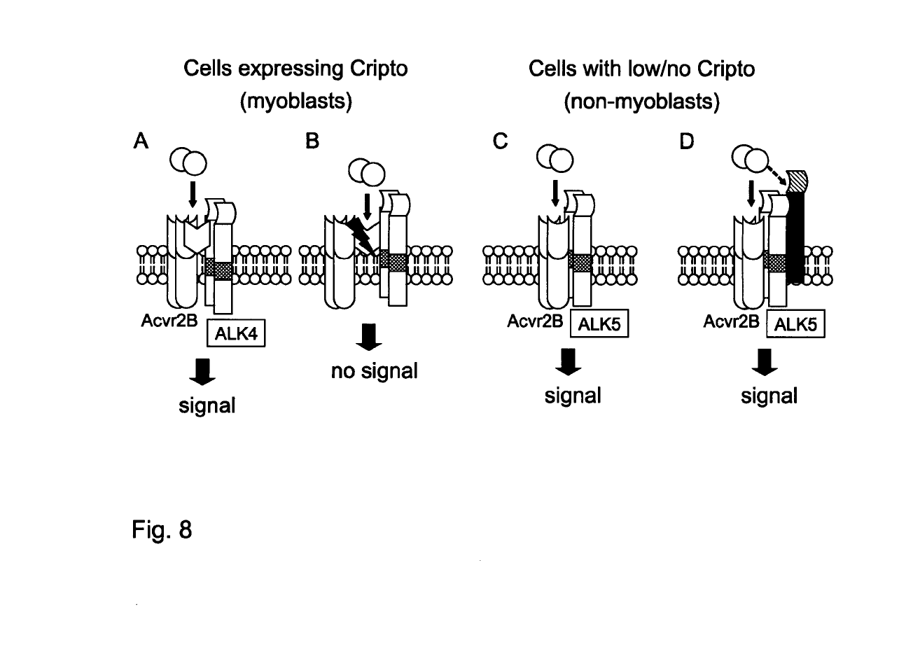

Based on the results, a mechanism was proposed by which myostatin signaling is regulated in different cell types. In the cells with significant expression of a co-receptor, such as, e.g., myogenic cells expressing TDGF1, the CFC domain of TDGF1 will be associated to ACVR1B, thereby transducing myostatin signaling (FIG. 8, Panel A). When TDGF1 is down-regulated, myostatin signaling was decreased, suggesting the importance of TDGF1 in this setting (FIG. 8, Panel B). Furthermore, both the EGF and CFC domains of TDGF1 seem to be important in modulating myostatin signaling. The repression observed upon TDGF1 knockdown in C2C12 cells was similar to that of ACVR1B knockdown, suggesting that compensation by TGFBR1 is unlikely to occur.

In the cell environment where TDGF1, or another ACVR1B co-receptor, is absent, such as in the non-myogenic cell panels tested, TGFBR1 seemed to be the main type I receptor for myostatin (FIG. 8, Panel C). The results also suggested that another co-receptor, betaglycan, might be modulating myostatin signaling in these non-myogenic cells (FIG. 8, Panel D).

### Example 8

### Antisense Oligonucleotides (AON)-Mediated Exon Skipping in ACVR1B and TGFBR1

In this study, the feasibility of specifically reducing ACVR1B or TGFBR1 signaling by means of exon skipping was examined. The sequences of the oligonucleotides used in the studies described herein are found in Table 1. The schematic overview of ACVR1B and TGFBR1 are depicted in FIG. 9A. Exon 2 of either ACVR1B or TGFBR1 encodes for the ligand binding domain. This exon was targeted with AON to generate transcripts without exon 2 and proteins lacking the ligand binding domain. Exon 6 was also targeted, which encodes part of the kinase domain. Targeting this exon with AON will generate out-of-frame transcript and terminate the protein translation prematurely due to the formation of stop codon in the subsequent exon.

Several AONs targeting ACVR1B exon 2 (A4E2) or exon 6 (A4E6) were transfected into C2C12 mouse myoblast cells. As shown in FIG. 9B, exon 6 of ACVR1B was removed upon A4E6 AON transfection in a dose-dependent manner. Subsequent sequence analysis showed that removal of exon 6 disrupted the open reading frame of Acvr1b and introduced a premature stop codon formation (not shown).

Furthermore, AONs were designed targeting exon 2 and exon 6 of TGFBR1, designated as A5E2 and A5E6 AONs, respectively, and performed screening in C2C12. RT-PCR and subsequent sequencing analysis showed successful removal of exon 2 or exon 6 of TGFBR1 using the A5E2 or A5E6 AONs in a dose-dependent manner (FIG. 9B and not shown). Unless noted, 500 nM was used as the optimum AON concentration in the further experiments.

A5E6 AON-transfected samples had significant decrease, approximately two-fold in the mRNA expression level (FIG. 10, Panel C). In addition to myoblast, other murine-derived cells were also tested, such as mesenchymal stem cells (C3H10 T1/2), endothelial cells (MEEC), fibroblasts (NIH/3T3) and preadipocytes (3T3-L1), and observed comparable skipping and knockdown levels.

In addition, TGFBR1 protein levels were also decreased upon A5E2 or A5E6 AONs treatment predominantly five days after transfection, while ACVR1B protein level could not be assessed due to the unavailability of specific antibody. These AONs allowed significant reduction of the expression of either ACVR1B or TGFBR1 with high specificity, thus not affecting the expression of other receptors.

TABLE 1

| AON | Targeting | Sequences (5′-3′) |
|---|---|---|
| A4E6 | ACVR1B exon 6 | UGGUGUCAGUGACCGCAUCAU |
| | | (SEQ ID NO: 1) |
| A4 | ACVR1B exon 6 | UGACUUCAAGUCUCGAUGAGC |
| | | (SEQ ID NO: 18) |
| A5E2 | TGFBR1 exon 2 | GCAGUGGUCCUGAUUGCAGCA |
| | | (SEQ ID NO: 3) |
| T2 | TGFBR1 exon 2 | CUUUGUACAGAGGUGGCAGAA |
| | | (SEQ ID NO: 19) |
| A5E6 | TGFBR1 exon 6 | AUCUGUGGCAGAAUCAUGUCU |
| | | (SEQ ID NO: 4) |
| T3 | TGFBR1 exon 6 | AUCUGUGGCAGAAUCAUGUCU |
| | | (SEQ ID NO: 4) |
| T4 | TGFBR1 exon 6 | UCUGUGGUUUGGAGCAAUAUC |
| | | (SEQ ID NO: 20) |
| ScrA4 | NA | UAUCUUGACCGCCUGAGAGGG |
| | | (SEQ ID NO: 6) |
| ScrA5 | NA | AUCUGCGAUCGGGACUUUGCA |
| | | (SEQ ID NO: 7) |
| M23D | Mouse | GGCCAAACCUCGGCUUACCU |
| (+02-18) | dystrophin | (SEQ ID NO: 8) |
| | exon 23 | |
| ViMo A4E6 | ACVR1B exon 6 | TCTATGGTGTCAGTGACCGCATCAT |
| | | (SEQ ID NO: 2) |
| ViMo A5E2 | TGFBR1 exon 2 | GCAGTGGTCCTGATTGCAGCAATAT |
| | | (SEQ ID NO: 5) |
| ViMo A5E6 | TGFBR1 exon 6 | TTGTATCTGTGGCAGAATCATGTCT |
| | | (SEQ ID NO: 21) |
| DMD | Dmd exon 23 | GGCCAAACCUCGGCUUACCU |
| | | (SEQ ID NO: 22) |

“ViMo” oligonucleotides have a morpholino backbone and equipped with a octa-guanidine dendrimer to enhance tissue uptake (GeneTools Inc.). Other oligonucleotides are 2′-O-methyl phosphorothioate modified.

T3 was effective in vitro, but exon skipping could not be detected in vivo.

A4, T2, T4, and A5E6 were all effective both in vitro and in vivo. A4E6, A5E2, ViMo A4E6, ViMo A5E2, and DMD showed even higher levels of exon skipping in vivo.

### Example 9

### TGFBR1AONs Inhibit TGF-Beta-Induced Smad2 Phosphorylation and CAGA Reporter Activity

It was hypothesized that removal of exon 2 will result in TGFBR1 lacking the ligand binding domain, whereas removal of exon 6 will truncate a significant part of the kinase domain. The later one might also be degraded via non-sense-mediated decay mechanism due to the formation of the premature stop codon. These products will thus be non-functional and their abilities to transduce TGF-beta signaling will be impaired. To elucidate this, each TGFBR1 AON was transfected together with (CAGA)₁₂ luciferase reporter constructs and stimulated with 1 ng/ml of TGF-beta. In C2C12 and C3H10 T1/2 cells, 80-90% repression of luciferase activity upon A5E2 or A5E6 AONs transfection was observed (FIGS. 11A and 11B). Comparable repression was achieved using 10 μM LY364947, a commercially available small molecule kinase inhibitor found to be an inhibitor of TGFBR1 and possibly other type I receptors (J. S. Sawyer et al. (2003), J. Med. Chem. 46(19) 3953-3956). Interestingly, when two TGFBR1 AONs A5E2 and A5E6 were combined, there was no additional decrease of luciferase activity, suggesting each TGFBR1AON has comparable potency and most likely achieved its maximum inhibitory effect. A4E6 AON did not abrogate TGF-beta-induced signaling, confirming that ACVR1B is not the type I receptor for TGF-beta.

### Example 10

### Effects of AON-Mediated ACVR1B and TGFBR1 Knockdown in Myostatin Signaling

As demonstrated in Example 2, ACVR1B is the predominant type I receptor for myostatin signal transduction process in myogenic cells, whereas TGFBR1 is used in the non-myogenic cells. Next, studies were conducted as to whether the effects shown using siRNA can be replicated using A4E6, A5E2 or A5E6. As shown in FIG. 12A, no significant decrease of myostatin-induced luciferase was observed in the C2C12 myoblast transfected with A5E2, A5E6 or combination of both. Conversely, when A4E6 AON was transfected, there was an approximate three-fold decrease of myostatin-induced luciferase in the C2C12. A more prominent decrease was not observed when both ACVR1B and TGFBR1AONs were co-transfected, suggesting a specific effect of ACVR1B knockdown.

A distinct effect was observed in the C3H10 T1/2 cells (FIG. 12B). Upon TGFBR1 knockdown with either the A5E2 or A5E6 AON, myostatin-induced luciferase decreased up to about 50%, whereas ACVR1B knockdown did not decrease the luciferase activity. Nevertheless, combination of both ACVR1B and TGFBR1 knockdown abrogated myostatin signaling significantly. This result demonstrates that exon-skipping oligonucleotides can be used to specifically knockdown ACVR1B or TGFBR1 signaling.

In addition, LY364947, which was described to be specific for TGFBR1 kinase activity, abrogated myostatin-induced CAGA luciferase in all cell types tested. It was thus suspected that these small molecule kinase inhibitors, at least in the concentration used, exhibited off-target effects for type I receptors other than TGFBR1, most likely ACVR1B.

Myostatin-induced Smad2 phosphorylation in the absence or presence of each AON (FIGS. 12C and 12D) was examined to confirm the preferential use of receptors. In line with the results from reporter assays, preferential use of ACVR1B for myostatin in C2C12 myoblasts was observed, whereas only TGFBR1 knockdown was able to abrogate Smad2 phosphorylation in C3H10 T1/2 and other non-myogenic cells.

Activin signaling is active in myoblasts and non-myoblasts (FIGS. 6D and 12E). This signaling is reduced upon knockdown of ACVR1B (FIGS. 6D and 12E).

### Example 11

### AON-Mediated ACVR1B or TGFBR1 Knockdown Enhances Myoblast Differentiation

After showing that the AONs exhibited strong potency in abrogating myostatin or TGF-beta signaling specifically, several biological functions were next examined in which TGF-beta or myostatin signaling play important roles, such as myoblast differentiation. C2C12 differentiation was induced by replacing the proliferation medium with low serum differentiation medium. Twenty-four hours after differentiation, 200 nM of A4E6, A5E2, A5E6 or control AONs were transfected and the differentiations were followed up to seven days. The degree of differentiation was measured by immunofluorescent staining detecting desmin/myosin heavy chain (FIG. 13). ACVR1B or TGFBR1 knockdown resulted in enhanced myotube formation. Formation of hypertrophic myotubes were observed starting from day 5 and more prominently at day 7.

### Example 12

### Local Administration of ACVR1B and TGFBR1AONs In Vivo

Potent inhibition of myostatin/TGF-beta pathways was demonstrated by AON-mediated exon skipping in ACVR1B or TGFBR1 in vitro. Next, the effects of these AONs in the mdx mice were examined (FIGS. 15 and 16). Specific knockdown of ACVR1B and TGFBR1 expressions with their respective AONs was observed. At four and ten days after injection, an increase in the expression of myogenic marker Myog upon ACVR1B exon skipping was observed, which was not observed upon TGFBR1 exon skipping, indicating that the ACVR1B AON specifically enhances myogenic differentiation. On the other hand, a decrease in the expression of the fibrotic marker SERPINE1 (Pai1) upon TGFBR1 exon skipping was observed, which was not observed upon ACVR1B exon skipping, indicating that the TGFBR1 AON specifically decreases fibrosis. Local inhibition of Smad2 activation was observed with TGFBR1 and ACVR1B AONs.

Further experiments are performed to assess the skip and knockdown levels after long-time treatment. The effects of each treatment on fibrosis and muscle fiber size are determined. The study is designed as follows.

Animals per group: N=6 (3 males, 3 females, 5 weeks old mdx)
Group 1: Saline control intravenously (iv) administered, weekly
Group 2: ViMo A4E6 iv 6 mg/kg weekly
Group 3: ViMo A5E2 iv 6 mg/kg weekly
Group 4: ViMo A4E6+A5E2 iv (6 mg+6 mg)/kg weekly
Group 5: DMSO ip daily
Group 6: LY364947 Intraperitoneal (ip) 5 mg/kg daily
Group 7: ViMo A4E6+DMD23 iv (6 mg+6 mg)/kg weekly
Group 8: ViMo A5E2+DMD23 iv (6 mg+6 mg)/kg weekly
Group 9: Triple combinations of A4E6+A5E2+DMD23

Duration of treatment: 6 weeks

Organs/tissues (tibialis anterior, soleus, quadriceps, gastrocnemius, heart, diaphragm, fat, lung, liver, and kidney) will be isolated for RNA, protein and histology analysis. Muscle, kidney and liver damage will be assessed by blood enzyme tests.

The results of similar experiments are described in Example 15.

### Example 13

### AON-Mediated ACVR1B or TGFBR1 Knockdown Inhibits TGF-Beta-Induced Fibroblast Proliferation and Fibrotic Responses

AONs were transfected into fibroblast cells and measured the proliferation rate upon stimulation with TGF-beta. 3T3-L1 preadipocytes with fibroblast properties were used to assess the effect of myostatin and TGF-beta in fibroblast proliferation (FIG. 14A). Both myostatin and TGF-beta stimulate fibroblast proliferation. The effect of knocking down ACVR1B and TGFBR1 signaling with AONs was also assessed. Both TGFBR1 knockdown and ACVR1B knockdown induces growth arrest of these fibroblast cells. Fibroblast differentiation was delayed by transfection with AONs against ACVR1B and TGFBR1, as apparent from reduced expression of the myofibroblast marker alpha-smooth muscle actin at three days but not five days after transfection (FIG. 14D).

### Example 14

### AON-Mediated TGFBR1 Knockdown in Human Cells

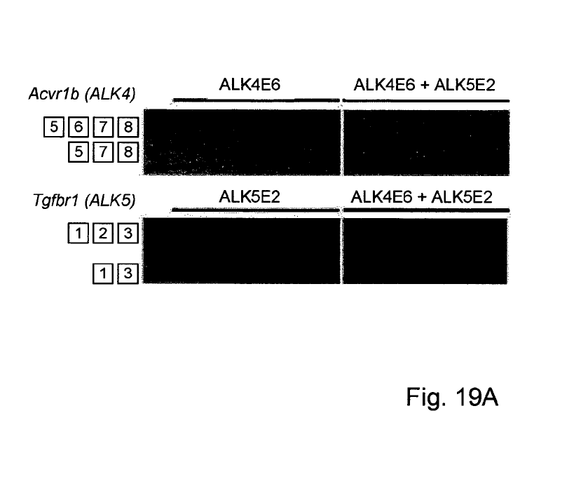

Immortalized human myoblast cells were transfected with 200 nM of control, hA5E2a or hA5E2b AONs (Table 2). RNA was isolated two days post-transfection for RT-PCR analysis (FIG. 18).

### Example 15

An investigation was performed as to whether a longer-term, systemic down-regulation of ACVR1b and TGFBR1 would also be feasible and beneficial. Based on the sequences of the existing A4E6 and A5E2 AONs with 2′-O-methyl phosphorothioate modifications, the morpholino counterparts were designed with octa guanidine conjugates (Vivo-Morpholino, further denoted as VM), because this chemistry has been described to enhance tissue uptake upon systemic administration.

Mdx mice were treated intravenously with 6 mg/kg VM targeting either ACVR1B (ALK4E6, A4E6) or TGFBR1 (ALK5E2, A5E2) once weekly for 6 weeks. Additional groups received either a cocktail of ALK4E6 and ALK5E2 or PBS as a control. The animals were sacrificed four days after the last injection and the triceps and diaphragm muscles were harvested.

RT-PCR analysis of the triceps showed prominent Acvr1b exon skipping in the animals treated with either the ALK4E6 AON or a combination of ALK4E6 and ALK5E2 AONs (FIG. 19A). Similarly, TGFBR1 exon skipping was observed in the ALK5E2 AON-treated mice as well as in the combination AON groups. Subsequent quantification using qPCR showed 30-40% knockdown of ACVR1B and 20-30% knockdown of TGFBR1 relative to the PBS-injected mice (FIG. 19B). The expression of Myog mRNA increased upon treatments, but significant increase was only detected in the combination of ALK4E6 and ALK5E2 group (FIG. 19C). While the expression of a fibrotic marker Col1a1 remained unchanged, alpha-SMA mRNA decreased in all groups, and the protein level decreased only in the combination group (FIGS. 19C and 19D). Decrease of SMAD2 phosphorylation levels in the triceps was only observed in the ALK5E2 AON-treated and combination AON groups, while the level in the ALK4E6 AON-treated triceps remained similar to control samples (FIG. 19D). This was accompanied by lower SERPINE1 protein levels in all AON-treated groups, although a significant decrease was only observed in the ALK4E6 AON-treated mice.

The diaphragms of the treated mdx mice were analyzed, since this muscle is known to show the most pronounced fibrosis in the mdx mouse model (Stedman et al., 1991). RT-PCR and QPCR analysis showed prominent exon skipping of ACVR1B and TGFBR1 (FIG. 20A), leading to ˜60-70% and 50-60% knockdown of ACVR1B and TGFBR1 full-length transcripts, respectively (FIG. 20B). Phosphorylated Smad2, SERPINE1 and aSMA levels decreased upon treatment with ACVR1B or TGFBR1AONs, and was, in particular, prominent in the combination treatment group (FIG. 20D), supporting the findings in the triceps. In contrast to the results observed in the triceps, Col1a1 transcripts decreased, whereas Myog remained unchanged (FIG. 20C).

Histological analysis using Sirius red staining as a measure for collagen deposition and fibrosis was performed. ALK4E6 AON-treated animals did not show any significant differences in fibrotic area (43.59±10.13) compared to the PBS-treated animals over (47.11±7.29%), whereas in the ALK5E2 and combination of ALK4E6 and ALK5E2 AONs-treated groups, the fibrotic percentage reduced to 33.38±5.94% and 29.92±4.90%, respectively (FIG. 21).

In conclusion, these results demonstrate the potency of ALK5E2 AON, either administered alone or in combination with ALK4E6 AON, to slow down or reverse the progression of fibrosis in diaphragm muscles in the mdx mice.

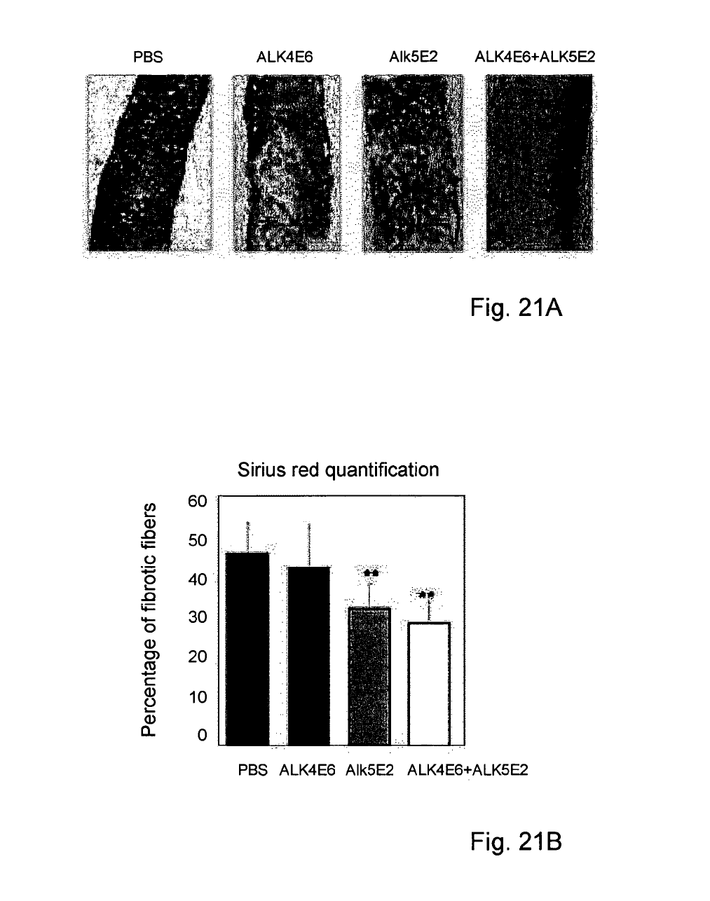

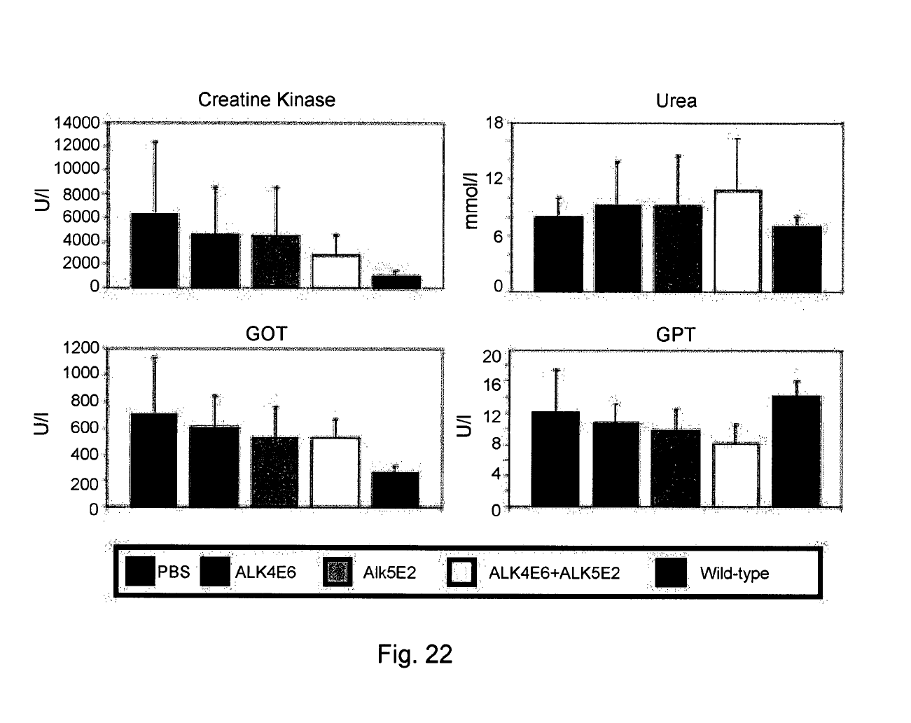

Serum was collected at the end of the treatment period. A trend was observed of a decrease in serum creatine kinase levels, which is one of the indicators of muscle damage, in AON-treated mdx mice toward the wild-type levels in the end of the study (FIG. 22). Although the differences of ALK4E6, ALK5E2 and combination AONs groups compared to control group were not significant due to the high variation between animals, this result suggested a general improvement in the muscle fiber integrity upon treatment, again especially in animals treated with the combination of ALK4E6 and ALK5E2 AONs. Finally, there was no significant increase in the liver enzymes such as glutamic-pyruvic transaminase (GPT) and glutamic-oxaloacetic transaminase (GOT), whereas the urea level was slightly elevated, although still within an acceptable range (FIG. 22). Overall, these blood enzyme parameters indicate that the treatment was well-tolerated by the animals.

### Example 16

The experiments showed that LY364947 could potently inhibit TGF-β- and myostatin-induced luciferase activity in vitro (FIGS. 11 and 12), but was unable to dissect between ALK4- or ALK5-mediated myostatin signaling, showing its poor specificity (FIG. 12). Also, it has been described that these compounds are relatively instable and, therefore, require more frequent administrations. The mdx mice were treated daily with the small inhibitor LY364947 but no changes were observed in the myogenic and fibrotic markers or in creatine kinase levels (FIG. 23). Although not elucidated further, it is possible that the bioavailability of this molecule was low and may require it to be employed with higher doses or more chronic treatment regimes.

TABLE 2

| AON | Targeting | AON sequence (5′ to 3′) |
|---|---|---|
| hA4E2a | ACVR1B exon 2 | UGGUGCACGCACACAGCAGAG |
| | | (SEQ ID NO: 23) |
| hA4E2b | ACVR1B exon 2 | CGUGUAGUUGGCCUGGAGGCA |
| | | (SEQ ID NO: 24) |
| hA4E2c | ACVR1B exon 2 | AGUUGGCCUGGAGGCAGCUG |
| | | (SEQ ID NO: 25) |
| hA4E2d | ACVR1B exon 2 | ACGUGUAGUUGGCCUGGAGG |
| | | (SEQ ID NO: 26) |
| hA4E2e | ACVR1B exon 2 | UCUCACACGUGUAGUUGGCCUGGAG |
| | | (SEQ ID NO: 27) |
| hA4E2f | ACVR1B exon 2 | UGCAGUAGUCAGUGUAGCAGCAGUG |
| | | (SEQ ID NO: 28) |
| hA4E2g | ACVR1B exon 2 | UCUGUCUCACACGUGUAGU |
| | | (SEQ ID NO: 29) |
| hA4E2h | ACVR1B exon 2 | UGUUGCAGUAGUCAGUGUAGCAGCA |
| | | (SEQ ID NO: 30) |
| hA4E6a | ACVR1B exon 6 | UGGUGUCAGUGACUGCAUCAU |
| | | (SEQ ID NO: 9) |
| hA4E6b | ACVR1B exon 6 | GUUUGGUCCCCACCCUCUGAUU |
| | | (SEQ ID NO: 10) |
| hA4E6c | ACVR1B exon 6 | GAUGAGCAAUUCCAGGCUUCC |
| | | (SEQ ID NO: 31) |
| hA4E6d | ACVR1B exon 6 | UCUGCUAUGGCACACAUGCCA |
| | | (SEQ ID NO: 32) |
| hA4E6e | ACVR1B exon 6 | GCAAUGUCAAUGGUGUCAGUGA |
| | | (SEQ ID NO: 33) |
| hA5E2a | TGFBR1 exon 2 | UGUACAGAGGUGGCAGAAACA |
| | | (SEQ ID NO: 11) |
| hA5E2b | TGFBR1 exon 2 | GCAAUGGUCCUGAUUGCAGCA |
| | | (SEQ ID NO: 12) |
| hA5E2c | TGFBR1 exon 2 | CAGAGACAAAGCAGAGCCCAUC |
| | | (SEQ ID NO: 34) |
| hA5E2d | TGFBR1 exon 2 | CUGUGGUCUCUGUGACAGAGACA |
| | | (SEQ ID NO: 35) |
| hA5E2e | TGFBR1 exon 2 | AUGCUGUUGUGUAUAACUUUGUC |
| | | (SEQ ID NO: 36) |
| hA5E2f | TGFBR1 exon 2 | AAUAUGUUGUAGUCACAGACC |
| | | (SEQ ID NO: 37) |
| hA5E6a | TGFBR1 exon 6 | UAUGAGCAAUGGCUGGCUUUC |
| | | (SEQ ID NO: 38) |
| hA5E6b | TGFBR1 exon 6 | UGAUUUCAAAUCUCUAUGAGCA |
| | | (SEQ ID NO: 39) |
| hA5E6c | TGFBR1 exon 6 | UGCCAGUCCUAAGUCUGCAAUA |
| | | (SEQ ID NO: 40) |
| hA5E6d | TGFBR1 exon 6 | AUAUCAAUGGUAUCUGUGGCU |
| | | (SEQ ID NO: 41) |
| hA5E6e | TGFBR1 exon 6 | CUGUGGUUUGGAGCAAUAUCAAU |
| | | (SEQ ID NO: 42) |
| hA5E6f | TGFBR1 exon 6 | UUCCCACUCUGUGGUUUGGAG |
| | | (SEQ ID NO: 43) |

### Materials and Methods

### Cell Cultures

Mouse myoblasts C2C12, mesenchymal stem cells C3H10 T1/2, pre-adipocytes 3T3-L1 and human embryonic kidney 293T were purchased from ATCC (Manassas, Va.). Mouse endothelial cells (MEEC) were kindly provided by Dr. Marie-Jose Goumans (LUMC). C2C12, 3T3-L1, MEEC and 293T cells were grown in DMEM with 10% FBS, 1% glucose and 2% glutamax (Gibco-BRL). C3H10 T1/2 cells were maintained in alpha-MEM medium supplemented with 10% FBS.

siRNA Transfection

ON-TARGETplus and ON-TARGETplus SMARTPOOLS® siRNA were purchased from Dharmacon (Thermo Scientific) and transfected using DHARMAFECT®DUO transfection reagents (Thermo Scientific) based on the manufacturer's instruction.

| siRNA targeting TGFBR1 (siTgfbr1): | |
|---|---|
| (SEQ ID NO: 14) | |
| Sense: | GGGCAGUUACUACAACAUAUU |
| (SEQ ID NO: 15) | |
| Antisense: | 5′-PUAUGUUGUAGUAACUGCCCUU |
| siRNA targeting ACVR1B (siAcvr1b): | |
| (SEQ ID NO: 16) | |
| Sense sequence: | ACUAUCAACUGCCGUAUUAUU |
| (SEQ ID NO: 17) | |
| Antisense sequence: | 5′-PUAAUACGGCAGUUGAUAGUUU |

### AON Transfection

AON with phosphorothioate backbones and 2′-O-methyl ribose modifications used in this study were synthesized and high-pressure liquid chromatography purified by Eurogentec, Belgium. The transfections were performed with LIPOFECTAMINE®2000 based on the manufacturer's protocol.

### RNA Isolation, Reverse-Transcriptase and Quantitative Real-Time PCR

RNA was isolated using NUCLEOSPIN® RNA II kit (Macherey-Nagel). cDNA was synthesized from 500 ng RNA using RevertAid H Minus M-MuLV Reverse Transcriptase (MBI Feiinentas) with 40 ng of random hexamer primer according to manufacturer's instructions. Quantitative PCR was performed in LIGHTCYCLER® 480 (Roche Diagnostics) with 2 μl of 10× diluted cDNA, 1 μl of 1 pmol/μl forward primer, 1 μl of 1 pmol/μl reverse primer and 4 μl of SENSIMIX® reagent (Bioline). Each measurement was performed in triplicate. Expressions of the genes of interest were normalized to housekeeping gene Gapdh and analyzed using the ΔΔCt method.

### Luciferase Reporter Assay

Cells were seeded at the density of 5×10³ cells/well in micro-clear white 96-well plates (Greiner Bio One). After 24 hours, the cells were transiently transfected with 100 ng of (CAGA)₁₂-Luc or (ARE)₃-Luc and 10 ng of pRL-CMV construct (Promega), with or without the indicated concentrations of siRNA using 0.6 μl of DHARMAFECT®DUO (Thermo Scientific). For 293T cells, 50 ng of A3-Luc, 50 ng of FAST1, 5 ng of pRL-CMV, 100 ng of Acv2b, 100 ng of ACVR1B and 100 ng of either the wild-type, ΔEGF or ΔCFC mutants TDGF1 were transfected. Cells were allowed to recover for 18 hours, followed by serum-starvation and stimulation with the indicated ligands for 8 hours. Following two washing steps, the cells were lysed using DualGlo Luciferase Assay Kit (Promega) and the luciferase signals were measured in the Multilabel Counter (PerkinElmer). The experiments were conducted in triplicate and repeated at least three times. The average measurements were shown.

### Smad Phosphorylation Assay and Western Blotting

Cells were seeded on a 6-well plate at the density of 3×10⁵ cells/well. Upon overnight serum starvation to establish the minimum basal level, the cells were stimulated with 1 ng/ml of TGF-beta (kindly provided by Ken Iwata at OSI Pharmaceuticals Inc., New York) or 500 ng/ml of myostatin (R&D Systems, Minneapolis) for 45 minutes. The cells were washed twice with GIBCO® HBSS and lysed with sample buffer containing 100 mM Tris-HCl pH 6.8, 4% w/v SDS, 0.1% w/v bromophenol blue, 20% v/v glycerol, 8% v/v beta-mercaptoethanol. Protein lysates were loaded and separated on 10% SDS-Page minigel and transferred onto nitrocellulose membranes (HyBond, Amersham). Membranes were blocked in 5% milk/TBS-TWEEN® for one hour and incubated in primary antibody in blocking buffer overnight at 4° C. After washing in TBS-TWEEN®, membranes were incubated in secondary antibody with IgG-HRP conjugate for one hour. Following 3× washing steps in TBS-TWEEN®, the detection was performed using SUPERSIGNAL® West Pico Chemiluminescent substrate (Thermo Scientific). The antibodies and dilutions were as follows: rabbit polyclonal anti phosphorylated Smad2 (1:1000, Ludwig Institute, Uppsala), mouse monoclonal anti actin (1:5000, Sigma), goat anti-rabbit and goat anti-mouse IgG-HRP (1:5000, SantaCruz).

### Myogenic Differentiation Assay

C2C12 cells were seeded on PERMANOX®-based LAB-TEK® chamber slides (Nunc, New York, USA) at a density of 150000 cells/0.4 ml proliferation medium/well. After 24 hours, the medium was changed into fusion medium (d0 fusion). After overnight fusion (d1 fusion), the cells were transfected with the designated siRNA or AONs using 0.6 μl of DHARMAFECT®DUO (Theimo Scientific) according to the manufacturer's instruction. The fusion progression was followed until seven days (d7 fusion). At d1, d3, d5 and d7, the fusion medium was removed and the cells were fixed with ice cold methanol for 5 minutes, dried at room temperature and the slides were stored at −20° C. prior to staining.

### Fibroblast Differentiation Assay

For the fibroblast differentiation experiment, 60-70% confluent primary fibroblasts, isolated from the gastrocnemius muscle were transfected with the indicated AONs and the medium was switched to DMEM with 2% FBS. Protein lysates were isolated after three and five days for immunoblotting analysis.

### Immunofluorescence Staining

The slides were incubated with PBS for 5 minutes and blocked for 30 minutes at RT with PBS/0.05% TWEEN® 20/0.05% Horse Serum. After performing washing steps twice with PBS, the cells were incubated for 1 hour with rabbit polyclonal anti-desmin (1:100, Santa Cruz) and mouse monoclonal anti myosin (MF20, 1:100, Hybridoma Bank Iowa), diluted in PBS/0.05% TWEEN® 20/0.05% FBS. After washing, the slides were incubated for 1 hour in the dark with secondary antibodies ALEXA FLUOR®-488 goat anti-mouse and -594 goat anti-rabbit (dilutions of 1:250 and 1:1000 in PBS/0.05% TWEEN®-20, Molecular Probes). Nuclei were stained using DAPI (0.2 ng/μl, Molecular Probes). Square cover slides were mounted to cover the cells using DAKO® mounting medium (Invitrogen) according to the manufacturer's instruction.

### AON Administrations

For the intramuscular administration, 2′-O-methyl phosphorothioate AONs were injected into the triceps of five-week-old mdx mice at the dose of 4×40 μg in 50 μl of physiological salt. At days 4 and 10 after the last injection, the mice were sacrificed and the triceps were isolated, snap frozen and sectioned for total RNA and protein isolation. For the systemic administration, the mice received 2×6 mg/kg of Vivo-Morpholino oligos (GeneTools, LLC, Oregon) in 100 μl of physiological salt via tail vein injection. Different organs were isolated four days after the last injection and RNA was isolated as described above. All the animal experiments were performed under the approval of the Animal Experimental Committee (DEC07195) of the LUMC.

For the systemic treatment, the animals received 6 mg/kg of vivo-morpholino modified AONs (GeneTools) intravenously via tail vein injections. The sequences are shown in Table 1. Control animals received sterile PBS at the equivalent volume of 100 μl. Each treatment group consisted of six mdx mice of five to six weeks old. The injections were performed once a week for six weeks and the animals were sacrificed four days after the last injection. Prior to sacrificing, blood samples were taken via the tail tip (see blood enzyme measurement section below). The tissues were harvested as mentioned above, except that part of the kidney was fixed in 4% PFA overnight and 70% ethanol until further use.

1. A compound that specifically reduces the amount or activity of TGFBR1 protein in a cell of an animal, wherein the compound is an oligonucleotide comprising between 15 and 40 nucleotides complementary to a TGFBR1 pre-mRNA, wherein said oligonucleotide binds TGFBR1 pre-mRNA and modulates splicing of said pre-mRNA.

2. The compound of claim 1, wherein said compound is used together with a second compound that specifically reduces the amount or activity of ACVR1B protein in a cell of an animal, wherein the second compound is an oligonucleotide comprising between 15 and 40 nucleotides complementary to an ACVR1B pre-mRNA, wherein said oligonucleotide binds ACVR1B pre-mRNA and modulates splicing of said pre-mRNA.

3. The composition of claim 1, wherein said composition further comprises a pharmaceutically acceptable excipient.

4.-7. (canceled)

8. A method for stimulating differentiation of a cell in the myogenic lineage comprising:
providing said cell with a compound that specifically reduces the amount or activity of ACVR1B protein in said cell, wherein the compound is an oligonucleotide comprising between 15 and 40 nucleotides complementary to an ACVR1B pre-mRNA, wherein said oligonucleotide binds ACVR1B pre-mRNA and modulates splicing of said pre-mRNA; and/or
providing said cell with a compound that specifically reduces the amount or activity of TGFBR1 protein in said cell, wherein the compound is an oligonucleotide comprising between 15 and 40 nucleotides complementary to a TGFBR1 pre-mRNA, wherein said oligonucleotide binds TGFBR1 pre-mRNA and modulates splicing of said pre-mRNA.

9. A method for silencing myostatin and/or activin signaling in a cell that is permissive to myostatin and/or activin signaling, the method comprising:
selecting a cell that expresses a) ACVR2A and/or ACVR2B, b) ACVR1B, and c) TGFBR1 and providing to said cell
a compound in an amount effective to specifically reduce the amount or activity of ACVR1B protein, wherein said oligonucleotide binds ACVR1B pre-mRNA and modulates splicing of said mRNA, and/or
an oligonucleotide in an amount effective to specifically reduce the amount or activity of TGFBR1 protein in said cell, wherein the oligonucleotide comprises between 15 and 40 nucleotides complementary to a TGFBR1 pre-mRNA, wherein said oligonucleotide binds TGFBR1 pre-mRNA and modulates splicing of said TGFBR1 pre-mRNA.

10. An oligonucleotide comprising between 15 and 40 nucleotides complementary to a TGFBR1 pre-mRNA, wherein said oligonucleotide binds TGFBR1 pre-mRNA and modulates splicing of said mRNA.

11. An oligonucleotide comprising between 15 and 40 nucleotides complementary to an ACVR1B pre-mRNA, wherein said oligonucleotide binds ACVR1B pre-mRNA and modulates splicing of said mRNA.

12. A nucleic acid molecule comprising the oligonucleotide of claim 10 operably linked to a promoter.

13. A composition or kit of parts comprising:
the oligonucleotide according to claim 10; and
a dystrophin exon-skipping nucleic acid.

14. A vector, comprising:
a) an oligonucleotide having between 15 and 40 nucleotides complementary to an ACVR1B pre-mRNA, wherein said oligonucleotide binds ACVR1B pre-mRNA and modulates splicing of said ACVR1B pre-mRNA, and/or
b) an oligonucleotide having between 15 and 40 nucleotides complementary to a TGFBR1 pre-mRNA, wherein said oligonucleotide binds TGFBR1 pre-mRNA and modulates splicing of said TGFBR1 pre-mRNA.

15. A cell comprising the oligonucleotide according to claim 10.

16. A method of inhibiting fibrosis development in an animal, the method comprising:
administering to the animal a first oligonucleotide comprising between 15 and 40 nucleotides complementary to TGFBR1 pre-mRNA,
wherein the first oligonucleotide binds TGFBR1 pre-mRNA and modulates splicing of TGFBR1 pre-mRNA in the animal so as to specifically reduce the effect of TGFBR1 protein in a cell of the animal.

17. The method according to claim 16, further comprising:
administering to the animal a second oligonucleotide comprising between 15 and 40 nucleotides complementary to ACVR1B pre-mRNA,
wherein the second oligonucleotide binds ACVR1B pre-mRNA and modulates splicing of ACVR1B pre-mRNA so as to specifically reduce the effect of ACVR1B protein in a cell of the animal.

18. A composition comprising:
a compound that specifically reduces the amount or activity of ACVR1B protein in a cell of an animal in an amount sufficient to inhibit, decrease, and/or prevent the development of fibrosis in the animal; and
a pharmaceutically acceptable excipient.

19. A method of promoting muscle growth or treating muscle loss or insufficient muscle growth in an animal, the method comprising:
administering to the animal an oligonucleotide comprising between 15 and 40 nucleotides complementary to TGFBR1 pre-mRNA,
wherein the oligonucleotide binds TGFBR1 pre-mRNA and modulates splicing of TGFBR1 pre-mRNA in the animal so as to specifically reduce the effect of TGFBR1 protein in a cell of the animal and promote muscle growth and/or reduce muscle loss or insufficient muscle growth in the animal.

20. The method according to claim 20, further comprising:
performing dystrophin exon-skipping in the animal.

21. A method of promoting muscle growth or treating muscle loss or insufficient muscle growth in an animal, the method comprising:
administering to the animal an oligonucleotide comprising between 15 and 40 nucleotides complementary to ACVR1B pre-mRNA,
wherein the oligonucleotide binds ACVR1B pre-mRNA and modulates splicing of ACVR1B pre-mRNA in the animal so as to specifically reduce the effect of ACVR1B protein in a cell of the animal and promote muscle growth and/or reduce muscle loss or insufficient muscle growth in the animal.

22. The method according to claim 21, further comprising:
performing dystrophin exon-skipping in the animal.

23. The method according to claim 21, wherein the effect of ACVR1B protein is reduced in a cell of the myogenic lineage, a muscle stem cell, a muscle fiber, or a cell expressing MYOD1.

24. The method according to claim 21, wherein the animal is suffering from muscular dystrophy.

25. The method according to claim 19, wherein the animal is suffering from muscular dystrophy.

26. A nucleic acid molecule comprising the oligonucleotide of claim 11 operably linked to a promoter.

27. A composition or kit of parts comprising:
the oligonucleotide of claim 11; and
a dystrophin exon-skipping nucleic acid.

28. The composition or kit of parts of claim 27, further comprising:
the oligonucleotide of claim 10.

29. The composition or kit of parts of claim 13, further comprising:
the oligonucleotide of claim 11.

30. The vector of claim 14, which is a viral vector.

31. A cell comprising the oligonucleotide of claim 11.
